(19) United States
(12) Patent Application Publication
(10) Pub. No.: US 2004/0115643 A1
(43) Pub. Date: Jun. 17, 2004

(54) Thermodynamic equilibrium extension of primers

(76) Inventors: Paul M. Lizardi, Wallingford, CT (US); Oleg G. Gribanov, Valadimir-Yurevets (RU)

Correspondence Address: NEEDLE & ROSENBERG, P.C., SUITE 1000, 999 PEACHTREE STREET, ATLANTA, GA, 30309-3915, US

(21) Appl. No.: 10/318,416

(22) Filed: Dec. 12, 2002

(51) Int. Cl. C12Q 1/68; C12P 19/34

(52) U.S. Cl. 435/6; 435/91.2

(57) ABSTRACT

Disclosed is a method and materials for amplifying nucleic acid sequences by limited primer extension. The disclosed method involves association of a primer with a template, extension of the primer for a short distance, termination of extension, and dissociation of the primer from the template, whereupon the events repeat with a new primer. The repeated association, extension, and dissociation of primers from a single template sequence results in amplification of the extended sequences. The termination of extension can be effected by a feature of the template sequence. The reaction can be carried under a single set of conditions, such as isothermal conditions, based on the thermodynamics of dissociation of the extended primers. The disclosed method is particularly suited to detection of nucleic acid sequences. Multiple sequences can be amplified and detected in the same reaction by targeting multiple sequences with extension primers.

### FIELD OF THE INVENTION

The disclosed invention is generally in the field of nucleic acid amplification and specifically in the area of primer extension.

### BACKGROUND OF THE INVENTION

Most methods used for nucleic acid detection are based on signal amplification or sequence amplification. Exiting approaches are driven by the need for reliable detection and accurate identification of minute amounts of target nucleic acids in biological samples. Among these approaches are the polymerase chain reaction (PCR), strand displacement amplification (SDA) (Guatelli, J. C, Whitfield, K. M., Kwoh, D. Y., Barringer, K. J., Richman, D. D. and Gingeras, T. R. (1990) Isothermal, in vitro amplification of nucleic acids by a multienzyme reaction modeled after retroviral replication. Proc. Natl. Acad. Sci. USA, 87:1874-1878), Qbeta Replicase amplification (Lizardi, P. M., Guerra, C. E., Lomeli, H., Tussie-Luna, I. and Kramer F. R. (1988) Exponential amplification of recombinant RNA hybridization probes. Bio/Technology, 6:1197-1202), self-sustained replication (3SR) (Guatelli, J. C, Whitfield, K. M., Kwoh, D. Y., Barringer, K. J., Richman, D. D. and Gingeras, T. R. (1990) Isothermal, in vitro amplification of nucleic acids by a multienzyme reaction modeled after retroviral replication. Proc. Natl. Acad. Sci. USA, 87:1874-1878), variants of nucleic acid sequence-based amplification (NASBA, TMA) (Kievits, T., van Gemen, B., van Strijp, D., Schukkink, R., Dircks, M., Adriaanse, H., Malek, L., Sooknanan, R. and Lens, P. (1991) NASBA isothermal enzymatic in vitro nucleic acid amplification optimized for the diagnosis of HIV-1 infection. J. Virol. Methods., 35:273-286), invader amplification assays (Lyamichev, V., Mast, A. L., Hall, J. G., Prudent, J. R., Kaiser, M. W., Takova, T., Kwiatkowski, R. W., Sander, T. J., de Arruda, M., Arco, D. A., Neri, B. P., Brow, M. A. (1999) Polymorphism identification and quantitative detection of genomic DNA by invasive cleavage of oligonucleotide probes. Nat. Biotechnol., 17:292-6), rolling circle amplification (RCA) (Lizardi, P. M., Huang, X., Zhu, Z., Bray-Ward, P, Thomas, D. and Ward, D. (1998) Mutation detection and single-molecule counting using isothermal rolling-circle amplification. Nature Genetics, 19:225-232), and loop-mediated isothermal amplification of DNA (Notomi, T., Okayama, H., Masubuchi, H., Yonekawa, T., Watanabe, K., Amino, N. and Hase, T. (2000) Loop-mediated isothermal amplification of DNA. Nucleic Acids Res., 28:E63). As signal detection methods improve, the need for very high amplification yields becomes less critical, and, paradoxically, it becomes attractive to use reactions with a lower amplification yield, but very high multiplexing potential. Highly multiplexed amplification reactions can be combined with microarrays, where multiplexed detection is only limited by array density. However, approaches such as PCR are of limited applicability to microarray-based readout because highly multiplexed amplification reactions often exhibit artifacts that lead to loss of efficiency and specificity (Edwards, M. C. and Gibbs, R. A. (1994) Multiplex PCR: advantages, development and applications. PCR Methods Appl., 3:S65-75). Microarray minisequencing is a highly multiplexed method for detecting single base changes, but its intrinsic sensitivity is low, requiring PCR-based pre-amplification.

There is a need for sensitive and precise DNA detection methods. As instrumentation develops, with consequent improvements in detection sensitivity, there is an increasing need for DNA amplification methods that yield linear, rather than exponential outputs. In other words, it becomes desirable to sacrifice amplification yield for more precise quantification.

### BRIEF SUMMARY OF THE INVENTION

Disclosed is a method and materials for amplifying nucleic acid sequences by limited primer extension. The disclosed method involves association of a primer with a template, extension of the primer for a short distance, termination of extension, and dissociation of the primer from the template, whereupon the events repeat with a new primer. The repeated association, extension, and dissociation of primers from a single template sequence results in amplification of the extended sequences. The termination of extension can be effected by a feature of the template sequence. The reaction can be carried under a single set of conditions, such as isothermal conditions, based on the thermodynamics of dissociation of the extended primers. At temperatures around the melting temperature (Tₘ) of the extended primer, including temperatures below the melting temperature, a significant fraction of the extended primer will be dissociated at any one time (put another way, each template sequence will be free of primer a fraction of the time). This allows a new, unextended primer to become associated with the template sequence and extended. The kinetics of the amplification can be improved by reducing the chance of reassociation of extended primers with the target sequence. This can be accomplished, for example, by providing a thermodynamically favored sink for the extended primers, by using an excess of unexteded primer over the number of extended primers that will be produced, or both. A thermodynamically favored sink can be, for example, a peptide nucleic acid complementary to the extended primer or ligation of the extended primer to another nucleic acid.

The unextended primers are referred to herein as extension primers. The template sequences are referred to herein as target templates. The extended primers are referred to herein as extended extension primers. The features of target templates that effect termination are referred to herein as a replication terminating feature. Replication terminating features can be, for example, 5′ ends, abasic nucleotides, and modified or derivatized nucleotides. Specific sequences in a nucleic acid sample can be amplified where a replication terminating feature is present or can be incorporated or created. 5′ ends can be created at specific sites by restriction endonucleases or by other sequence-specific nucleic acid cleaving enzymes or techniques. The sequences amplified also depend on the extension primers; only those sequences targeted by an extension primer (and adjacent to a replication terminating feature) will be amplified. This allows specific sequences to be targeted for amplification. Flexibility in the location of replication terminating features allows flexibility in targeting sequences. If a targeted sequence is not present (or if there is no adjacent replication terminating feature), the sequence will not be amplified.

The disclosed method is particularly suited to detection of nucleic acid sequences. By amplifying specific sequences targeted by extension primers and adjacent to replication terminating features, extended extension primers are produced incorporating the targeted sequences. By detecting the extended extension primers, the corresponding targeted sequence is detected. If a targeted sequence is not present (or if there is no adjacent replication terminating feature), the sequence will not be amplified, no extended extension primer corresponding to that sequence will be produced or detected, and thus the sequence will not be detected.

Multiple sequences can be amplified in the same reaction by targeting multiple sequences with extension primers. Likewise, multiple sequences can be detected via production of their corresponding extended extension primers. Multiplex detection is facilitated by the different sequences of different extension primers. For example, the extended extension primers can be hybridized to detection probes. Detection probes can also serve as sink for extended extension primers during the amplification reaction. In this way, sequences can be detected during the amplification reaction. Such simultaneous amplification and detection can be facilitated by using detection probes associated with a substrate. Multiplex detection can be facilitated by an array of detection probes with different detection probes at different locations of a substrate.

The extended extension probes can be used for any purpose such as detection of sequences, as probes, primers, or oligonucleotides for use in arrays, chips, and kits. The extended extension primers can be used in any method or technique that involves oligonucleotides.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a diagram of an example of the disclosed method (top) and a diagram of an example of dideoxy chain termination and termination facilitated by the replication terminating features 5′ end and modified nucleotide residues. In each case, the target template is SEQ ID NO:16, the extension primer is nucleotides 1-16 of SEQ ID NO:17, and the extended extension primer is SEQ ID NO:17.

FIGS. 2A, 2B, and 2C are diagrams of three examples of target templates and extension primers and the results of primer extension at various temperatures using different dideoxynucleotides for chain termination. FIG. 2A used a synthetic template (38nt; SEQ ID NO:18). FIGS. 2B and 2C used HCV cDNA (201nt). The sequence shown is SEQ ID NO:18. Extensions were produced by incubation at 70° C., 74° C. or 78° C. for 2 hours in reaction mixtures contained 4 nM templates, 1 μM primer, one of the four ddNTP (50 μM) and tree other dNTPs (200 μM). The primer is SEQ ID NO:20.

FIGS. 3A and 3B are diagrams of a target template (SEQ ID NO:21) and different extension primers and the results of primer extension terminated by the 5′ end of the template. Extension was produced by incubation at 70° C. for 1 hour in reaction mixture contained 4 nM templates, 1 μM primer and 0.08 U/μl Vent(exo⁻), 0.02 U/ μl DeepVent(exo⁻), 0.05 U/μl Taq or 0.16 U/μl ThermoSequenase. The primers are all nucleotides 19-34 of SEQ ID NO:23, except the last primer, which is nucleotides 1-34 of SEQ ID NO:23.

FIGS. 4A, 4B, and 4C are diagrams of examples of three target templates and extension primers and the results of primer extension terminated by deoxyuridine residues. Extension was carried out at 72° C. or 76° C. (FIGS. 4A and 4B, respectively) or at 70° C., 74° C. and 78° C. (FIG. 4C) for 1 hour with 0.02U/μl of DeepVent(exo⁻) DNA polymerase. Template1 is SEQ ID NO:24 and primer1 is SEQ ID NO:28. Template2 is SEQ ID NO:25 and primer2 is SEQ ID NO:29. Template3 is SEQ ID NO:26 and primer3 is SEQ ID NO:30.

FIG. 5 is a graph of log(SQ/ASQ) for 50 different addresses (representing 50 different target sequences).

FIG. 6 is a graph of log(SQ/ASQ) for 50 different addresses (representing 50 different target sequences).

FIG. 7 is a diagram showing an example of a stem-loop detection probe and its use.

FIG. 8 is a diagram of an example of the disclosed method involving preparation of target template by deoxyuridine-specific fragmentation followed by primer extension terminated by the 5′ ends of the templates. The fragment of HCV RNA is SEQ ID NO:15. The primer for fragmentation N1 is 1-20 of SEQ ID NO:2. The primer for fragmentation N2 is 1-18 of SEQ ID NO:3. The extended primers are SEQ ID NO:2 and SEQ ID NO:3. Template N1 is 20-47 of SEQ ID NO:2. Template N2 is 18-58 of SEQ ID NO:3. The primer for TEX T1 is 1-18 of SEQ ID NO:4. The primer for TEX T2 is 1-15 of SEQ ID NO:5. The extended TEX primers are SEQ ID NO:4 and SEQ ID NO:5.

### DETAILED DESCRIPTION OF THE INVENTION

The disclosed method and compositions may be understood more readily by reference to the following detailed description of particular embodiments and the Example included therein and to the Figures and their previous and following description.

Disclosed is a method and materials for amplifying nucleic acid sequences by limited primer extension. The disclosed method involves association of a primer with a template, extension of the primer for a short distance, termination of extension, and dissociation of the primer from the template, whereupon the events repeat with a new primer. The repeated association, extension, and dissociation of primers from a single template sequence results in amplification of the extended sequences. The termination of extension can be effected by a feature of the template sequence. The reaction can be carried under a single set of conditions, such as isothermal conditions, based on the thermodynamics of dissociation of the extended primers. At temperatures around the melting temperature of the extended primer, including temperatures below the melting temperature, a significant fraction of the extended primer will be dissociated at any one time (put another way, each template sequence will be free of primer a fraction of the time). This allows a new, unextended primer to become associated with the template sequence and extended. The kinetics of the amplification can be improved by reducing the chance of reassociation of extended primers with the target sequence. This can be accomplished, for example, by providing a thermodynamically favored sink for the extended primers, by using an excess of unexteded primer over the number of extended primers that will be produced, or both. A thermodynamically favored sink can be, for example, a peptide nucleic acid complementary to the extended primer or ligation of the extended primer to another nucleic acid.

The extended extension probes can be used for any purpose such as detection of sequences, as probes, primers, or oligonucleotides for use in arrays, chips, and kits. The extended extension primers can be used in any method or technique that involves oligonucleotides.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular extension primer is disclosed and discussed and a number of modifications that can be made to a number of molecules including the extension primer are discussed, each and every combination and permutation of extension primer and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Unextended primers are referred to herein as extension primers. The template sequences are referred to herein as target templates. The extended primers are referred to herein as extended extension primers. The features of target templates that effect termination are referred to herein as a replication terminating feature. Replication terminating features can be, for example, 5′ ends, abasic nucleotides, and modified or derivatized nucleotides. Specific sequences in a nucleic acid sample can be amplified where a replication terminating feature is present or can be incorporated or created. 5′ ends can be created at specific sites by restriction endonucleases or by other sequence-specific nucleic acid cleaving enzymes or techniques. The sequences amplified also depend on the extension primers; only those sequences targeted by an extension primer (and adjacent to a replication terminating feature) will be amplified. This allows specific sequences to be targeted for amplification. Flexibility in the location of replication terminating features allows flexibility in targeting sequences. If a targeted sequence is not present (or if there is no adjacent replication terminating feature), the sequence will not be amplified.

Multiple sequences can be amplified in the same reaction by targeting multiple sequences with extension primers. Likewise, multiple sequences can be detected via production of their corresponding extended extension primers. Multiplex detection is facilitated by the different sequences of different extension primers. For example, the extended extension primers can be hybridized to detection probes. Detection probes can also serve as sink for extended extension primers during the amplification reaction. In this way, sequences can be detected during the amplification reaction. Such simultaneous amplification and detection can be facilitated by using detection probes associated with a substrate. Multiplex detection can be facilitated by an array of detection probes with different detection probes at different locations of a substrate.

A. Extension Primers

Extension primers are primers that interact with target templates and are extended using the target template as the template. Thus, extension primers are designed to interact with target templates and to be extendible. The extension primers can interact with target templates in a nucleotide sequence-specific manner (by base pairing or hybridization, for example). Where there are different extension primers and/or different target templates, the nucleotide sequence-specific interaction generally occurs only between particular extension primers and particular target templates. That is, extension primers can be specific to particular target templates based on the specificity of the nucleotide sequence-specific interaction. Extension primers having such a relationship with a given target template can be said to correspond to the target template. Similarly, the target templates that have such a relationship with a given extended extension primer can be said to correspond to the extended extension primers.

Useful extension primers comprise oligonucleotides, oligonucleotide analogs, or a combination. Extension primers can comprise a target complement portion and, optionally, a non-target complement portion. The target complement portion is complementary to sequence in a target template referred to as the primer complement region of the target template. Once an extension primer is extended it is an extended extension primer (that is, the extension primer with the nucleotides added by the extension of the primer). Both the extension primer and the extended extension primer are complementary to the target template, with the extended extension primer having more complementary nucleotides.

Extension primers will be extended from their location of interaction with the target template to a replication terminating feature in the target template. The length of this extension is determined by the location of interaction and the location of the replication terminating feature. Extension primers can be extended in the disclosed method by any number of nucleotides that allows the cycle of interaction, extension, and dissociation to take place. Generally, this feature is related to the relative melting temperatures of the extension primer and of the extended extension primer that results.

Design of extension primers generally should take into account the relative melting temperatures of the extension primer and of the extended extension primer that results. Thus, the length of the target complement portions of extension primers generally can be adjusted in design based on the composition of the target complement portion and the length and composition of the extension portion of the extended extension primer. The length of the extension portion (that is, the length of extension) is determined by the distance between the 3′ end of the extension primer when interacting with the target template and this distance can be adjusted during design by locating the 3′ end of the extension primer appropriately. It should be understood that, if desired, target templates can be designed to work with particular extension primers. Thus, the length and composition of the primer complement region of target templates can be adjusted based on the relative melting temperatures of the extension primer and of the extended extension primer that results. The extended extension primer that results from extension of a given extension primer can be said to correspond to the extension primer. Similarly, the extension primer that is extended to form a given extended extension primer can be said to correspond to the extended extension primer.

Design of extension primers should also take into account the location of replication terminating features. That is, the primer complement region of the target templates (for which the target complement portions of the extension primers are designed) can be chosen based on the location of replication terminating features. Alternatively, the location of replication terminating features can be introduced at particular locations in nucleic acids (and the extension primers designed based on these locations). The distance between the primer complement region of the target template and the replication terminating feature generally determines the number of nucleotides added during extension and thus affects the melting temperature of the extended extension primer.

The target complement portion of an extension primer can be designed to be complementary to particular sequences of interest within a nucleic acid sample. Generally, extension primers should be designed to interact with sequences that are of interest (for example, sequences where mutations may be present) and that are adjacent to replication terminating features. Where particular sequences of interest are to be targeted, replication terminating features (such as 5′ ends) can be introduced in or adjacent to the sequence of interest and an extension primer designed to interact with the sequence of interest. Where flexibility is possible, the sequence of an extension primer can be chosen such that it is not significantly complementary to any other portion of the target template. With consideration of other extension primer design factors, the target complement portion of an extension primer can be any length that supports specific and stable interaction between the target complement portion of the extension primer and the primer complement region of the target template.

Extension primers can contain additional sequence (and/or other features) that is not complementary to any part of the target template. This sequence is referred to as the non-target complement portion of the extension primer. The non-target complement portion of the primer, if present, can facilitate, for example, detection, immobilization, or separation of the primers. The non-target complement portion of an extension primer may be any length, but is generally 1 to 100 nucleotides long, and preferably 4 to 8 nucleotides long. The non-target complement portion is generally at the 5′ end of the primer. To aid in detection and quantitation of extended extension primers, extension primers can include one or more detection labels. Extension primers can, but need not, include non-target complement portion and/or features other than the target complement portion. Thus, for example, extension primers may consist of a target complement portion, extension primers may comprise a target complement portion, and extension primers may comprise nucleotides where the nucleotides consist of a target complement portion.

Extension primers can have one or more modified nucleotides. Such extension primers are referred to herein as modified extension primers. Some forms of modified extension primers, such as RNA/2′-O-methyl RNA chimeric extension primers, have a higher melting temperature than DNA primers. Also, extension primers made of RNA will be exonuclease resistant. These and other nucleotide modifications are described more extensively elsewhere herein.

1. Extension Primer Melting Temperature

The melting temperatures (Tₘ) of extension primers are related to the dissociation constants (k_{d}) for the extension primers, and the melting temperature and dissociation constant are related to the hybrid stability of extension primers. The same relationships exist for extended extension primers. The relationship between these characteristics in the extension primers and these characteristics in their cognate extended extension primers can affect the amplification reaction, and thus can be a factor in the design of extension primers. As the Tₘ of the extended extension primer increases relative to the Tₘ of the extension primer, the dissociation rate of the extended extension primer decreases relative to the dissociation rate of the extension primer. This relative decrease will generally increase the ratio of extended extension primer/target template (that is, extended extension primer bound to target template) to extension primer/target template (that is, the extension primer bound to target template) for a given concentration of extended extension primer and extension primer. Because the amplification reaction proceeds more efficiently, in general, when the ratio of extended extension primer/target template to extension primer/target template is lower, it is desirable to limit the difference in melting temperature of the extended extension primer and the extension primer. It should be understood that differences in the concentration of the extension primer and extended extension primers will also affect the ratio of extended extension primer/target template to extension primer/target template, with higher extension primer concentrations and lower extended extension primer concentrations decreasing the ratio as discuss more fully elsewhere herein. This effect can be used to counteract some of the effect of the difference in melting temperature of the extension primer and extended extension primer.

Unless otherwise indicated to the contrary, reference herein to the melting temperature of an extension primer refers to the melting temperature of the extension primer when interacting with a target template. Because the target complement portion of an extension primer interacts with the target template, and because extension primers can include portions and features other than a target complement portion, it should be understood that reference to the melting temperature of an extension primer refers to the melting temperature of the target complement portion of the extension primer when interacting with the target template. Similarly, because the primer complement region of a target template interacts with the extension primer, and because target templates can include portions, regions, and features other than a primer complement region, it should be understood that reference to the melting temperature of an extension primer refers to the melting temperature of the extension primer when interacting with the primer complement region of the target template. It also should be understood that reference to the melting temperature of an extension primer refers to the melting temperature of the target complement of the extension primer when interacting with the primer complement region of the target template. The interaction of an extension primer and a target template can be described in any or all of these ways and should be understood to encompass all of these descriptions. The melting temperature of an extension primer when interacting with a sequence or component other than a target template is sometimes relevant and may be referred to explicitly herein. For example, the melting temperature of an extension primer when interacting with a detection probe or anchor probe may be referred to explicitly as such.

The melting temperature of extension primers and other disclosed components and oligonucleotides can be calculated using known formulas and principles of thermodynamics (see, for example, Santa Lucia, “A unified view of polymer, dumbbell, and oligonucleotide DNA nearest-neighbor thermodynamics” Proc Natl Acad Sci USA. 95(4):1460-5 (1998), Santa Lucia et al., Biochemistry 35:3555-3562 (1996); Freier et al., Proc. Natl. Acad. Sci. USA 83:9373-9377 (1986); Breslauer et al., Proc. Natl. Acad. Sci. USA 83:3746-3750 (1986)). Such calculations are known. Such calculations are based on the assignment of entropy and enthalpy terms to each nearest neighbor dinucleotide in the DNA sequence. For example, the sequence AGTCA comprises four nearest neighbors, AG, GT, TC, CA. Using the equations described by Freier et al. (1986) and Breslauer et al. (1986), and the parameter table described by Santa Lucia et al. (1996, 1998), one calculates the free energy for the duplex, and then, using the free energy value, the predicted melting temperature can be calculated. Melting temperatures can also be determined empirically by measuring the relevant interaction at different temperatures. As used herein, calculated melting temperature refers to a melting temperature calculated using formulas and principles as discussed above. Measured melting temperature refers to a melting temperature determined empirically. Where the melting temperatures of extension primers and other components are to be compared, the melting temperatures can be determined using the same technique or calculation.

i. Percent Difference in Melting Temperature

For extension primer design purposes, the difference in melting temperature can be expressed as the percent difference in melting temperature of the extension primer and the extended extension primer. Although the following description refers to the percent difference in melting temperature of extension primers and extended extension primers, this description should also be understood to disclose and describe the percent difference in melting temperature of the target complement portion of extension primers and extended extension primers, the percent difference in melting temperature of extension primers and the extended target complement portion of extended extension primers, the percent difference in melting temperature of the target complement portion of extension primers and the extended target complement portion extended extension primers, the percent difference in melting temperature of extension primers when interacting with target templates and extended extension primers, the percent difference in melting temperature of extension primers and extended extension primers when interacting with target templates, the percent difference in melting temperature of extension primers when interacting with target templates and extended extension primers when interacting with target templates, the percent difference in melting temperature of extension primers when interacting with target templates to which the extension primers correspond and extended extension primers, the percent difference in melting temperature of extension primers and extended extension primers when interacting with target templates to which the extended extension primers correspond, and the percent difference in melting temperature of extension primers when interacting with target templates to which the extension primers correspond and extended extension primers when interacting with target templates to which the extended extension primers correspond. The percent difference in melting temperature of the extension primer and extended extension primer can be, for example, about 1 percent, about 2 percent, about 3 percent, about 4 percent, about 5 percent, about 6 percent, about 7 percent, about 8 percent, about 9 percent, about 10 percent, about 11 percent, about 12 percent, about 13 percent, about 14 percent, about 15 percent, about 16 percent, about 17 percent, about 18 percent, about 19 percent, or about 20 percent. The percent difference in melting temperature of the extension primer and extended extension primer can be, for example, less than about 1 percent, less than about 2 percent, less than about 3 percent, less than about 4 percent, less than about 5 percent, less than about 6 percent, less than about 7 percent, less than about 8 percent, less than about 9 percent, less than about 10 percent, less than about 11 percent, less than about 12 percent, less than about 13 percent, less than about 14 percent, less than about 15 percent, less than about 16 percent, less than about 17 percent, less than about 18 percent, less than about 19 percent, or less than about 20 percent.

The percent difference in melting temperature of the extension primer and extended extension primer can be, for example, from about 1 percent to about 2 percent, from about 1 percent to about 3 percent, from about 1 percent to about 4 percent, from about 1 percent to about 5 percent, from about 1 percent to about 6 percent, from about 1 percent to about 7 percent, from about 1 percent to about 8 percent, from about 1 percent to about 9 percent, from about 1 percent to about 10 percent, from about 1 percent to about 11 percent, from about 1 percent to about 12 percent, from about 1 percent to about 13 percent, from about 1 percent to about 14 percent, from about 1 percent to about 15 percent, from about 1 percent to about 16 percent, from about 1 percent to about 17 percent, from about 1 percent to about 18 percent, from about 1 percent to about 19 percent, or from about 1 percent to about 20 percent. The percent difference in melting temperature of the extension primer and extended extension primer can be, for example, from about 2 percent to about 3 percent, from about 2 percent to about 4 percent, from about 2 percent to about 5 percent, from about 2 percent to about 6 percent, from about 2 percent to about 7 percent, from about 2 percent to about 8 percent, from about 2 percent to about 9 percent, from about 2 percent to about 10 percent, from about 2 percent to about 11 percent, from about 2 percent to about 12 percent, from about 2 percent to about 13 percent, from about 2 percent to about 14 percent, from about 2 percent to about 15 percent, from about 2 percent to about 16 percent, from about 2 percent to about 17 percent, from about 2 percent to about 18 percent, from about 2 percent to about 19 percent, or from about 2 percent to about 20 percent.

The percent difference in melting temperature of the extension primer and extended extension primer can be, for example, from about 3 percent to about 4 percent, from about 3 percent to about 5 percent, from about 3 percent to about 6 percent, from about 3 percent to about 7 percent, from about 3 percent to about 8 percent, from about 3 percent to about 9 percent, from about 3 percent to about 10 percent, from about 3 percent to about 11 percent, from about 3 percent to about 12 percent, from about 3 percent to about 13 percent, from about 3 percent to about 14 percent, from about 3 percent to about 15 percent, from about 3 percent to about 16 percent, from about 3 percent to about 17 percent, from about 3 percent to about 18 percent, from about 3 percent to about 19 percent, or from about 3 percent to about 20 percent. The percent difference in melting temperature of the extension primer and extended extension primer can be, for example, from about 4 percent to about 5 percent, from about 4 percent to about 6 percent, from about 4 percent to about 7 percent, from about 4 percent to about 8 percent, from about 4 percent to about 9 percent, from about 4 percent to about 10 percent, from about 4 percent to about 11 percent, from about 4 percent to about 12 percent, from about 4 percent to about 13 percent, from about 4 percent to about 14 percent, from about 4 percent to about 15 percent, from about 4 percent to about 16 percent, from about 4 percent to about 17 percent, from about 4 percent to about 18 percent, from about 4 percent to about 19 percent, or from about 4 percent to about 20 percent.

The percent difference in melting temperature of the extension primer and extended extension primer can be, for example, from about 5 percent to about 6 percent, from about 5 percent to about 7 percent, from about 5 percent to about 8 percent, from about 5 percent to about 9 percent, from about 5 percent to about 10 percent, from about 5 percent to about 11 percent, from about 5 percent to about 12 percent, from about 5 percent to about 13 percent, from about 5 percent to about 14 percent, from about 5 percent to about 15 percent, from about 5 percent to about 16 percent, from about 5 percent to about 17 percent, from about 5 percent to about 18 percent, from about 5 percent to about 19 percent, or from about 5 percent to about 20 percent. The percent difference in melting temperature of the extension primer and extended extension primer can be, for example, from about 6 percent to about 7 percent, from about 6 percent to about 8 percent, from about 6 percent to about 9 percent, from about 6 percent to about 10 percent, from about 6 percent to about 11 percent, from about 6 percent to about 12 percent, from about 6 percent to about 13 percent, from about 6 percent to about 14 percent, from about 6 percent to about 15 percent, from about 6 percent to about 16 percent, from about 6 percent to about 17 percent, from about 6 percent to about 18 percent, from about 6 percent to about 19 percent, or from about 6 percent to about 20 percent.

The percent difference in melting temperature of the extension primer and extended extension primer can be, for example, from about 7 percent to about 8 percent, from about 7 percent to about 9 percent, from about 7 percent to about 10 percent, from about 7 percent to about 11 percent, from about 7 percent to about 12 percent, from about 7 percent to about 13 percent, from about 7 percent to about 14 percent, from about 7 percent to about 15 percent, from about 7 percent to about 16 percent, from about 7 percent to about 17 percent, from about 7 percent to about 18 percent, from about 7 percent to about 19 percent, or from about 7 percent to about 20 percent. The percent difference in melting temperature of the extension primer and extended extension primer can be, for example, from about 8 percent to about 9 percent, from about 8 percent to about 10 percent, from about 8 percent to about 11 percent, from about 8 percent to about 12 percent, from about 8 percent to about 13 percent, from about 8 percent to about 14 percent, from about 8 percent to about 15 percent, from about 8 percent to about 16 percent, from about 8 percent to about 17 percent, from about 8 percent to about 18 percent, from about 8 percent to about 19 percent, or from about 8 percent to about 20 percent. The percent difference in melting temperature of the extension primer and extended extension primer can be, for example, from about 9 percent to about 10 percent, from about 9 percent to about 11 percent, from about 9 percent to about 12 percent, from about 9 percent to about 13 percent, from about 9 percent to about 14 percent, from about 9 percent to about 15 percent, from about 9 percent to about 16 percent, from about 9 percent to about 17 percent, from about 9 percent to about 18 percent, from about 9 percent to about 19 percent, or from about 9 percent to about 20 percent.

The percent difference in melting temperature of the extension primer and extended extension primer can be, for example, from about 10 percent to about 11 percent, from about 10 percent to about 12 percent, from about 10 percent to about 13 percent, from about 10 percent to about 14 percent, from about 10 percent to about 15 percent, from about 10 percent to about 16 percent, from about 10 percent to about 17 percent, from about 10 percent to about 18 percent, from about 10 percent to about 19 percent, or from about 10 percent to about 20 percent. The percent difference in melting temperature of the extension primer and extended extension primer can be, for example, from about 11 percent to about 12 percent, from about 11 percent to about 13 percent, from about 11 percent to about 14 percent, from about 11 percent to about 15 percent, from about 11 percent to about 16 percent, from about 11 percent to about 17 percent, from about 11 percent to about 18 percent, from about 11 percent to about 19 percent, or from about 11 percent to about 20 percent. The percent difference in melting temperature of the extension primer and extended extension primer can be, for example, from about 12 percent to about 13 percent, from about 12 percent to about 14 percent, from about 12 percent to about 15 percent, from about 12 percent to about 16 percent, from about 12 percent to about 17 percent, from about 12 percent to about 18 percent, from about 12 percent to about 19 percent, or from about 12 percent to about 20 percent.

The percent difference in melting temperature of the extension primer and extended extension primer can be, for example, from about 13 percent to about 14 percent, from about 13 percent to about 15 percent, from about 13 percent to about 16 percent, from about 13 percent to about 17 percent, from about 13 percent to about 18 percent, from about 13 percent to about 19 percent, or from about 13 percent to about 20 percent. The percent difference in melting temperature of the extension primer and extended extension primer can be, for example, from about 14 percent to about 15 percent, from about 14 percent to about 16 percent, from about 14 percent to about 17 percent, from about 14 percent to about 18 percent, from about 14 percent to about 19 percent, or from about 14 percent to about 20 percent. The percent difference in melting temperature of the extension primer and extended extension primer can be, for example, from about 15 percent to about 16 percent, from about 15 percent to about 17 percent, from about 15 percent to about 18 percent, from about 15 percent to about 19 percent, or from about 15 percent to about 20 percent.

The percent difference in melting temperature of the extension primer and extended extension primer can be, for example, from about 16 percent to about 17 percent, from about 16 percent to about 18 percent, from about 16 percent to about 19 percent, or from about 16 percent to about 20 percent. The percent difference in melting temperature of the extension primer and extended extension primer can be, for example, from about 17 percent to about 18 percent, from about 17 percent to about 19 percent, from about 17 percent to about 20 percent, from about 18 percent to about 19 percent, from about 18 percent to about 20 percent, or from about 19 percent to about 20 percent.

ii. Difference in Melting Temperature

The melting temperature of extension primers can also be expressed in terms of the difference in the melting temperature of their cognate extended extension primer. Although the following description refers to the melting temperature of extension primers in relation to the melting temperature of extended extension primers, this description should also be understood to disclose and describe the melting temperature of the target complement portion of extension primers in relation to the melting temperature of extended extension primers, the melting temperature of extension primers in relation to the melting temperature of the extended target complement portion of extended extension primers, the melting temperature of the target complement portion of extension primers in relation to the melting temperature of the extended target complement portion extended extension primers, the melting temperature of extension primers when interacting with target templates in relation to the melting temperature of extended extension primers, the melting temperature of extension primers in relation to the melting temperature of extended extension primers when interacting with target templates, the melting temperature of extension primers when interacting with target templates in relation to the melting temperature of extended extension primers when interacting with target templates, the melting temperature of the target complement portion of extension primers in relation to the melting temperature of extended extension primers to which the extension primers correspond, the melting temperature of extension primers in relation to the melting temperature of the extended target complement portion of extended extension primers to which the extension primers correspond, and the melting temperature of the target complement portion of extension primers in relation to the melting temperature of the extended target complement portion extended extension primers to which the extension primers correspond. The melting temperature of the extension primers can be, for example, about 2° C. to about 25° C., about 3° C. to about 25° C., about 4° C. to about 25° C., about 5° C. to about 25° C., about 6° C. to about 25° C., about 7° C. to about 25° C., about 8° C. to about 25° C., about 9° C. to about 25° C., about 10° C. to about 25° C., about 11° C. to about 25° C., about 12° C. to about 25°about 13° C. to about 25° C., about 14° C. to about 25° C., about 15° C. to about 25° C., about 16° C. to about 25° C., about 17° C. to about 25° C., about 18° C. to about 25° C., about 19° C. to about 25° C., about 20° C. to about 25° C., about 21° C. to about 25° C., about 22° C. to about 25° C., about 23° C. to about 25° C., or about 24° C. to about 25° C. lower than the melting temperature of the extended extension primer. The melting temperature of the extension primers can be, for example, about 2° C. to about 24° C., about 3° C. to about 24° C., about 4° C. to about 24° C., about 5° C. to about 24° C., about 6° C. to about 24° C., about 7° C. to about 24° C., about 8° C. to about 24° C., about 9° C. to about 24° C., about 10° C. to about 24° C., about 11° C. to about 24° C., about 12° C. to about 24° C., about 13° C. to about 24° C., about 14° C. to about 24° C., about 15° C. to about 24° C., about 16° C. to about 24° C., about 17° C. to about 24° C., about 18° C. to about 24° C., about 19° C. to about 24° C., about 20° C. to about 24° C., about 21° C. to about 24° C., about 22° C. to about 25° C., or about 23° C. to about 24° C. lower than the melting temperature of the extended extension primer.

The melting temperature of the extension primers can be, for example, about 2° C. to about 23° C., about 3° C. to about 23° C., about 4° C. to about 23° C., about 5° C. to about 23° C., about 6° C. to about 23° C., about 7° C. to about 23° C., about 8° C. to about 23° C., about 9° C. to about 23° C., about 10° C. to about 23° C., about 11° C. to about 23° C., about 12° C. to about 23° C., about 13° C. to about 23° C., about 14° C. to about 23° C., about 15° C. to about 23° C., about 16° C. to about 23° C., about 17° C. to about 23° C., about 18° C. to about 23° C., about 19° C. to about 23° C., about 20° C. to about 23° C., about 21° C. to about 23° C., or about 22° C. to about 23° C. lower than the melting temperature of the extended extension primer. The melting temperature of the extension primers can be, for example, about 2° C. to about 22° C., about 3° C. to about 22° C., about 4° C. to about 22° C., about 5° C. to about 22° C., about 22° C., about 7° C. to about 22° C., about 8° C. to about 22° C., about 9° C. to about 22° C., about 10° C. to about 22° C., about 11° C. to about 22° C., about 12° C. to about 22° C., about 13° C. to about 22° C., about 14° C. to about 22° C., about 15° C. to about 22° C., about 16° C. to about 22° C., about 17° C. to about 22° C., about 18° C. to about 22° C., about 19° C. to about 22° C., about 20° C. to about 22° C., or about 21° C. to about 22° C. lower than the melting temperature of the extended extension primer.

The melting temperature of the extension primers can be, for example, about 2° C. to about 21° C., about 3° C. to about 21° C., about 4° C. to about 21° C., about 5° C. to about 21° C., about 6° C. to about 21° C., about 7° C. to about 21° C., about 8° C. to about 21° C., about 9° C. to about 21° C., about 10° C. to about 21° C., about 11° C. to about 21° C., about 12° C. to about 21° C., about 13° C. to about 21° C., about 14° C. to about 21° C., about 15° C. to about 21° C., about 16° C. to about 21° C., about 17° C. to about 21° C., about 18° C. to about 21° C., about 19° C. to about 21° C., or about 20° C. to about 21° C. lower than the melting temperature of the extended extension primer. The melting temperature of the extension primers can be, for example, about 2° C. to about 20° C., about 3° C. to about 20° C., about 4° C. to about 20° C., about 5° C. to about 20° C., about 6° C. to about 20° C., about 7° C. to about 20° C., about 8° C. to about 20° C., about 9° C. to about 20° C., about 10° C. to about 20° C., about 11° C. to about 20° C., about 12° C. to about 20° C., about 13° C. to about 20° C., about 14° C. to about 20° C., about 15° C. to about 20° C., about 16° C. to about 20° C., about 17° C. to about 20° C., about 18° C. to about 20° C., or about 19° C. to about 20° C. lower than the melting temperature of the extended extension primer.

The melting temperature of the extension primers can be, for example, about 2° C. to about 19° C., about 3° C. to about 19° C., about 4° C. to about 19° C., about 5° C. to about 19° C., about 6° C. to about 19° C., about 7° C. to about 19° C., about 8° C. to about 19° C., about 9° C. to about 19° C., about 10° C. to about 19° C., about 11° C. to about 19° C., about 12° C. to about 19° C., about 13° C. to about 19° C., about 14° C. to about 19° C., about 15° C. to about 19° C., about 16° C. to about 19° C., about 17° C. to about 19° C., or about 18° C. to about 19° C. lower than the melting temperature of the extended extension primer. The melting temperature of the extension primers can be, for example, about 2° C. to about 18° C., about 3° C. to about 18° C., about 4° C. to about 18° C., about 5° C. to about 18° C., about 6° C. to about 18° C., about 7° C. to about 18° C., about 8° C. to about 18° C., about 9° C. to about 18° C., about 10° C. to about 18° C., about 11° C. to about 18° C., about 12° C. to about 18° C., about 13° C. to about 18° C., about 14° C. to about 18° C., about 15° C. to about 18° C., about 16° C. to about 18° C., or about 17° C. to about 18° C. lower than the melting temperature of the extended extension primer.

The melting temperature of the extension primers can be, for example, about 2° C. to about 17° C., about 3° C. to about 17° C., about 4° C. to about 17° C., about 5° C. to about 17° C., about 6° C. to about 17° C., about 7° C. to about 17° C., about 8° C. to about 17° C., about 9° C. to about 17° C., about 10° C. to about 17° C., about 11° C. to about 17° C., about 12° C. to about 17° C., about 13° C. to about 17° C., about 14° C. to about 17° C., about 15° C. to about 17° C., or about 16° C. to about 17° C. lower than the melting temperature of the extended extension primer. The melting temperature of the extension primers can be, for example, about 2° C. to about 16° C., about 3° C. to about 16° C., about 4° C. to about 16° C., about 5° C. to about 16° C., about 6° C. to about 16° C., about 7° C. to about 16° C., about 8° C. to about 16° C., about 9° C. to about 16° C., about 10° C. to about 16° C., about 11° C. to about 16° C., about 12° C. to about 16° C., about 13° C. to about 16° C., about 14° C. to about 16° C., or about 15° C. to about 16° C. lower than the melting temperature of the extended extension primer.

The melting temperature of the extension primers can be, for example, about 2° C. to about 15° C., about 3° C. to about 15° C., about 4° C. to about 15° C., about 5° C. to about 15° C., about 6° C. to about 15° C., about 7° C. to about 15° C., about 8° C. to about 15° C., about 9° C. to about 15° C., about 10° C. to about 15° C., about 11° C. to about 15° C., about 12° C. to about 15° C., about 13° C. to about 15° C., or about 14° C. to about 15° C. lower than the melting temperature of the extended extension primer. The melting temperature of the extension primers can be, for example, about 2° C. to about 14° C., about 3° C. to about 14° C., about 4° C. to about 14° C., about 5° C. to about 14° C., about 6° C. to about 14° C., about 7° C. to about 14° C., about 8° C. to about 14° C., about 9° C. to about 14° C., about 10° C. to about 14° C., about 11° C. to about 14° C., about 12° C. to about 14° C., or about 13° C. to about 14° C. lower than the melting temperature of the extended extension primer. The melting temperature of the extension primers can be, for example, about 2° C. to about 13° C., about 3° C. to about 13° C., about 7° C. to about 4° C. to about 13° C., about 5° C. to about 13° C., about 6° C. to about 13° C., about 7° C. to about 13° C., about 8° C. to about 13° C., about 9° C. to about 13° C., about 10° C. to about 13° C., about 11° C. to about 13° C., or about 12° C. to about 13° C. lower than the melting temperature of the extended extension primer.

The melting temperature of the extension primers can be, for example, about 2° C. to about 12° C., about 3° C. to about 12° C., about 4° C. to about 12° C., about 5° C. 12° C., about 6° C. to about 12° C., about 7° C. to about 12° C., about 8° C. to about 12° C., about 9° C., about 12° C., about 10° C. to about 12° C., or about 11° C. to about 12° C. lower than the melting temperature of the extended extension primer. The melting temperature of the extension primers can be, for example, about 2° C. to about 11° C., about 3° C. to about 11° C., about 4° C. to about 11° C., about 5° C. to about 1° C., about 6° C. to about 11° C., about 7° C. to about 11° C., about 8° C. to about 11° C., about 9° C. to about 11° C., or about 10° C. to about 11° C. lower than the melting temperature of the extended extension primer. The melting temperature of the extension primers can be, for example, about 2° C. to about 10° C., about 3° C. to about 10° C., about 4° C. to about 10° C., about 5° C. to about 10° C., about 6° C. to about 10° C., about 7° C. to about 10° C., about 8° C. to about 10° C., or about 9° C. to about 10° C. lower than the melting temperature of the extended extension primer.

The melting temperature of the extension primers can be, for example, about 2° C. to about 9° C., about 3° C. to about 9° C., about 4° C. to about 9° C., about 5° C. to about 9° C., about 6° C. to about 9° C., about 7° C. to about 9° C., or about 8° C. to about 9° C. lower than the melting temperature of the extended extension primer. The melting temperature of the extension primers can be, for example, about 2° C. to about 8° C., about 3° C. to about 8° C., about 4° C. to about 8° C., about 5° C. to about 8° C., about 6° C. to about 8° C., or about 7° C. to about 8° C. lower than the melting temperature of the extended extension primer. The melting temperature of the extension primers can be, for example, about 2° C. to about 7° C., about 3° C. to about 7° C., about 4° C. to about 7° C., about 5° C. to about 7° C., or about 6° C. to about 7° C. lower than the melting temperature of the extended extension primer. The melting temperature of the extension primers can be, for example, about 2° C. to about 6° C., about 3° C. to about 6° C., about 4° C. to about 6° C., about 5° C. to about 6° C., 2° C. to about 5° C., about 3° C. to about 5° C., about 4° C. to about 5° C., 2° C. to about 4° C., about 3° C. to about 4° C., or 2° C. to about 3° C. lower than the melting temperature of the extended extension primer.

The melting temperature of the extension primers can be, for example, about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., or about 25° C. lower than the melting temperature of the extended extension primer. The melting temperature of the extension primers can be, for example, 2° C., 3° C., 4° C., 5° C., 6° C., 7° C., 8° C., 9° C., 10° C., 11° C., 12° C., 13° C., 14° C., 15° C. 16° C., 17° C., 18° C., 19° C., 20° C., 21° C. 22° C., 23° C., 24° C., or 25° C. lower than the melting temperature of the extended extension primer. All of the above relationships also can be described in terms of the melting temperature of the extended extension primer being higher than the melting temperature of extension primers, and such alternative descriptions are contemplated and should be considered specifically disclosed.

The melting temperature of extension primers can also be expressed in terms of the difference from the temperature at which the extension primers and target templates are incubated. Although the following description refers to the melting temperature of extension primers in relation to the temperature at which the extension primers and target templates are incubated, this description should also be understood to disclose and describe the melting temperature of the target complement portion of extension primers in relation to the temperature at which the extension primers and target templates are incubated, the melting temperature of extension primers when interacting with target templates in relation to the temperature at which the extension primers and target templates are incubated, and the melting temperature of extension primers when interacting with target templates to which the extension primers correspond in relation to the temperature at which the extension primers and target templates are incubated. The melting temperature of the extension primer can be, for example, about 5° C. to about 20° C., about 5° C. to about 19° C., about 5° C. to about 18° C., about 5° C. to about 17° C., about 5° C. to about 16° C., about 5° C. to about 15° C., about 5° C. to about 14° C., about 5° C. to about 13° C., about 5° C. to about 12° C., about 5° C. to about 11° C., about 5° C. to about 10° C., about 5° C. to about 9° C., about 5° C. to about 8° C., about 5° C. to about 7° C., or about 5° C. to about 6° C. lower than the temperature at which the extension primers and target templates are incubated.

The melting temperature of the extension primer can be, for example, about 6° C. to about 20° C., about 6° C. to about 19° C., about 6° C. to about 18° C., about 6° C. to about 17° C., about 6° C. to about 16° C., about 6° C. to about 15° C., about 6° C. to about 14° C., about 6° C. to about 13° C., about 6° C. to about 12° C., about 6° C. to about 11° C., about 6° C. to about 10° C., about 6° C. to about 9° C., about 6° C. to about 8° C., or about 6° C. to about 7° C. lower than the temperature at which the extension primers and target templates are incubated. The melting temperature of the extension primer can be, for example, about 7° C. to about 20° C., about 7° C. to about 19° C., about 7° C. to about 18° C., about 7° C. to about 17° C., about 7° C. to about 16° C., about 7° C. to about 15° C., about 7° C. to about 14° C., about 7° C. to about 13° C., about 7° C. to about 12° C., about 7° C. to about 11° C., about 7° C. to about 10° C., about 7° C. to about 9° C., or about 7° C. to about 8° C. lower than the temperature at which the extension primers and target templates are incubated.

The melting temperature of the extension primer can be, for example, about 8° C. to about 20° C., about 8° C. to about 19° C., about 8° C. to about 18° C., about 8° C. to about 17° C., about 8° C. to about 16° C., about 8° C. to about 15° C., about 8° C. to about 14° C., about 8° C. to about 13° C., about 8° C. to about 12° C., about 8° C. to about 11° C., about 8° C. to about 10° C., or about 8° C. to about 9° C. lower than the temperature at which the extension primers and target templates are incubated. The melting temperature of the extension primer can be, for example, about 9° C. to about 20° C., about 9° C. to about 19° C., about 9° C. to about 18° C., about 9° C. to about 17° C., about 9° C. to about 16° C., about 9° C. to about 15° C., about 9° C. to about 14° C., about 9° C. to about 13° C., about 9° C. to about 12° C., about 9° C. to about 11° C., or about 9° C. to about 10° C. lower than the temperature at which the extension primers and target templates are incubated.

The melting temperature of the extension primer can be, for example, about 10° C. to about 20° C., about 10° C. to about 19° C., about 10° C. to about 18° C., about 10° C. to about 17° C., about 10° C. to about 16° C., about 10° C. to about 15° C., about 10° C. to about 14° C., about 10° C. to about 13° C., about 10° C. to about 12° C., or about 10° C. to about 11° C. lower than the temperature at which the extension primers and target templates are incubated. The melting temperature of the extension primer can be, for example, about 11° C. to about 20° C., about 11° C. to about 19° C., about 11° C. to about 18° C., about 11° C. to about 17° C., about 11° C. to about 16° C., about 11° C. to about 15° C., about 11° C. to about 14° C., about 11° C. to about 13° C., or about 1°° C. to about 12° C. lower than the temperature at which the extension primers and target templates are incubated. The melting temperature of the extension primer can be, for example, about 12° C. to about 20° C., about 12° C. to about 19° C., about 12° C. to about 18° C., about 12° C. to about 17° C., about 12° C. to about 16° C., about 12° C. to about 15° C., about 12° C. to about 14° C., or about 12° C. to about 13° C. lower than the temperature at which the extension primers and target templates are incubated.

The melting temperature of the extension primer can be, for example, about 13° C. to about 20° C., about 13° C. to about 19° C., about 13° C. to about 18° C., about 13° C. to about 17° C., about 13° C. to about 16° C., about 13° C. to about 15° C., or about 13° C. to about 14° C. lower than the temperature at which the extension primers and target templates are incubated. The melting temperature of the extension primer can be, for example, about 14° C. to about 20° C., about 14° C. to about 19° C., about 14° C. to about 18° C., about 14° C. to about 17° C., about 14° C. to about 16° C., or about 14° C. to about 15° C. lower than the temperature at which the extension primers and target templates are incubated. The melting temperature of the extension primer can be, for example, about 15° C. to about 20° C., about 15° C. to about 19° C., about 15° C. to about 18° C., about 15° C. to about 17° C., or about 15° C. to about 16° C. lower than the temperature at which the extension primers and target templates are incubated.

The melting temperature of the extension primer can be, for example, about 16° C. to about 20° C., about 16° C. to about 19° C., about 16° C. to about 18° C., about 16° C. to about 17° C., 17° C. to about 20° C., about 17° C. to about 19° C., about 17° C. to about 18° C., about 18° C. to about 20° C., about 18° C. to about 19° C., or about 19° C. to about 20° C. lower than the temperature at which the extension primers and target templates are incubated. The melting temperature of the extension primer can be, for example, about 20° C., about 19° C., about 18° C., about 17° C., about 16° C., about 15° C., about 14° C., about 13° C., about 12° C., about 11° C., about 10° C., about 9° C., about 8° C., about 7° C., about 6° C., or about 5° C. lower than the temperature at which the extension primers and target templates are incubated. The melting temperature of the extension primer can be, for example, 20° C., 19° C., 18° C., 17° C., 16° C., 15° C., 14° C., 13° C., 12° C., 11° C., 10° C., 9° C., 8° C., 7° C., 6° C., or 5° C. lower than the temperature at which the extension primers and target templates are incubated. All of the above relationships also can be described in terms of the temperature at which the extension primer and target templates are incubated being higher than the melting temperature of the extension primer, and such alternative descriptions are contemplated and should be considered specifically disclosed.

iii. Melting Temperature

The melting temperature of extension primers can also be expressed in absolute terms. However, the principles discussed above and elsewhere herein, such as the temperature of incubation and its useful relationship to the melting temperature of extension primers, should be considered in designing the melting temperature of extension primers. Although the following description refers to the melting temperature of extension primers, this description should also be understood to disclose and describe the melting temperature of the target complement portion of extension primers, the melting temperature of extension primers when interacting with target templates, and the melting temperature of extension primers when interacting with target templates to which the extension primers correspond. The melting temperature of the extension primer can be, for example, about 48° C. to about 69° C., about 48° C. to about 67° C., about 48° C. to about 65° C., about 48° C. to about 63° C., about 48° C. to about 61° C., about 48° C. to about 60° C., about 48° C. to about 59° C., about 48° C. to about 58° C., about 48° C. to about 57° C., about 48° C. to about 56° C., about 48° C. to about 55° C., about 48° C. to about 53° C., or about 48° C. to about 51° C. The melting temperature of the extension primer can be, for example, about 51° C. to about 69° C., about 51° C. to about 67° C., about 51° C. to about 65° C., about 51° C. to about 63° C., about 51° C. to about 61° C., about 51° C. to about 60° C., about 51° C. to about 59° C., about 51° C. to about 58° C., about 51° C. to about 57° C., about 51° C. to about 56° C., about 51° C. to about 55° C., or about 51° C. to about 53° C.

The melting temperature of the extension primer can be, for example, about 53° C. to about 69° C., about 53° C. to about 67° C., about 53° C. to about 65° C., about 53° C. to about 63° C., about 53° C. to about 61° C., about 53° C. to about 60° C., about 53° C. to about 59° C., about 53° C. to about 58° C., about 53° C. to about 57° C., about 53° C. to about 56° C., or about 53° C. to about 55° C. The melting temperature of the extension primer can be, for example, about 55° C. to about 69° C., about 55° C. to about 67° C., about 55° C. to about 65° C., about 55° C. to about 63° C., about 55° C. to about 61° C., about 55° C. to about 60° C., about 55° C. to about 59° C., about 55° C. to about 58° C., about 55° C. to about 57° C., or about 55° C. to about 56° C. The melting temperature of the extension primer can be, for example, about 56° C. to about 69° C., about 56° C. to about 67° C., about 56° C. to about 65° C., about 56° C. to about 63° C., about 56° C. to about 61° C., about 56° C. to about 60° C., about 56° C. to about 59° C., about 56° C. to about 58° C., or about 56° C. to about 57° C.

The melting temperature of the extension primer can be, for example, about 57° C. to about 69° C., about 57° C. to about 67° C., about 57° C. to about 65° C., about 57° C. to about 63° C., about 57° C. to about 61° C., about 57° C. to about 60° C., about 57° C. to about 59° C., or about 57° C. to about 58° C. The melting temperature of the extension primer can be, for example, about 58° C. to about 69° C., about 58° C. to about 67° C., about 58° C. to about 65° C., 58° C. to about 59° C. The melting temperature of the extension primer can be, for example, about 59° C. to about 69° C., about 59° C. to about 67° C., about 59° C. to about 65° C., about 59° C. to about 63° C., about 59° C. to about 61° C., or about 59° C. to about 60° C.

The melting temperature of the extension primer can be, for example, about 60° C. to about 69° C., about 60° C. to about 67° C., about 60° C. to about 65° C., about 60° C. to about 63° C., about 60° C. to about 61° C., about 61° C. to about 69° C., about 61° C. to about 67° C., about 61° C. to about 65° C., about 61° C. to about 63° C., about 63° C. to about 69° C., about 63° C. to about 67° C., about 63° C. to about 65° C., about 65° C. to about 69° C., about 65° C. to about 67° C., or about 67° C. to about 69° C. The melting temperature of the extension primer can be, for example, about 69° C., about 67° C., about 65° C., about 63° C., about 61° C., about 60° C., about 59° C., about 58° C., about 57° C., about 56° C., about 55° C., about 51° C., or about 48° C. The melting temperature of the extension primer can be, for example, 69° C., 67° C., 65° C., 63° C., 61° C., 60° C., 59° C., 58° C., 57° C., 56° C., 55° C., 53° C., 51° C., or 48° C.

2. Primer Extension

The design of extension primers can also be expressed in terms of the number of nucleotides added by extension. However, the effect of the added nucleotides on the melting temperature of the resulting extended extension primer, and thus the effect on the relationship of the melting temperature of the extension primer and the extended extension primer, should be considered in designing extension primers. The extension primer can be extended, for example, 3 to 20 nucleotides, 3 to 19 nucleotides, 3 to 18 nucleotides, 3 to 17 nucleotides, 3 to 16 nucleotides, 3 to 15 nucleotides, 3 to 14 nucleotides, 3 to 13 nucleotides, 3 to 12 nucleotides, 3 to 11 nucleotides, 3 to 10 nucleotides, 3 to 9 nucleotides, 3 to 8 nucleotides, 3 to 7 nucleotides, 3 to 6 nucleotides, 3 to 5 nucleotides, or 3 to 4 nucleotides. The extension primer can be extended, for example, 4 to 20 nucleotides, 4 to 19 nucleotides, 4 to 18 nucleotides, 4 to 17 nucleotides, 4 to 16 nucleotides, 4 to 15 nucleotides, 4 to 14 nucleotides, 4 to 13 nucleotides, 4 to 12 nucleotides, 4 to 11 nucleotides, 4 to 10 nucleotides, 4 to 9 nucleotides, 4 to 8 nucleotides, 4 to 7 nucleotides, 4 to 6 nucleotides, or 4 to 5 nucleotides.

The extension primer can be extended, for example, 5 to 20 nucleotides, 5 to 19 nucleotides, 5 to 18 nucleotides, 5 to 17 nucleotides, 5 to 16 nucleotides, 5 to 15 nucleotides, 5 to 14 nucleotides, 5 to 13 nucleotides, 5 to 12 nucleotides, 5 to 11 nucleotides, 5 to 10 nucleotides, 5 to 9 nucleotides, 5 to 8 nucleotides, 5 to 7 nucleotides, or 5 to 6 nucleotides. The extension primer can be extended, for example, 6 to 20 nucleotides, 6 to 19 nucleotides, 6 to 18 nucleotides, 6 to 17 nucleotides, 6 to 16 nucleotides, 6 to 15 nucleotides, 6 to 14 nucleotides, 6 to 13 nucleotides, 6 to 12 nucleotides, 6 to 11 nucleotides, 6 to 10 nucleotides, 6 to 9 nucleotides, 6 to 8 nucleotides, or 6 to 7 nucleotides. The extension primer can be extended, for example, 7 to 20 nucleotides, 7 to 19 nucleotides, 7 to 18 nucleotides, 7 to 17 nucleotides, 7 to 16 nucleotides, 7 to 15 nucleotides, 7 to 14 nucleotides, 7 to 13 nucleotides, 7 to 12 nucleotides, 7 to 11 nucleotides, 7 to 10 nucleotides, 7 to 9 nucleotides, or 7 to 8 nucleotides.

The extension primer can be extended, for example, 8 to 20 nucleotides, 8 to 19 nucleotides, 8 to 18 nucleotides, 8 to 17 nucleotides, 8 to 16 nucleotides, 8 to 15 nucleotides, 8 to 14 nucleotides, 8 to 13 nucleotides, 8 to 12 nucleotides, 8 to 11 nucleotides, 8 to 10 nucleotides, or 8 to 9 nucleotides. The extension primer can be extended, for example, 9 to 20 nucleotides, 9 to 19 nucleotides, 9 to 18 nucleotides, 9 to 17 nucleotides, 9 to 16 nucleotides, 9 to 15 nucleotides, 9 to 14 nucleotides, 9 to 13 nucleotides, 9 to 12 nucleotides, 9 to 11 nucleotides, or 9 to 10 nucleotides. The extension primer can be extended, for example, 10 to 20 nucleotides, 10 to 19 nucleotides, 10 to 18 nucleotides, 10 to 17 nucleotides, 10 to 16 nucleotides, 10 to 15 nucleotides, 10 to 14 nucleotides, 10 to 13 nucleotides, 10 to 12 nucleotides, or 10 to 11 nucleotides. The extension primer can be extended, for example, 11 to 20 nucleotides, 11 to 19 nucleotides, 11 to 18 nucleotides, 11 to 17 nucleotides, 11 to 16 nucleotides, 11 to 15 nucleotides, 11 to 14 nucleotides, 11 to 13 nucleotides, or 11 to 12 nucleotides.

The extension primer can be extended, for example, 12 to 20 nucleotides, 12 to 19 nucleotides, 12 to 18 nucleotides, 12 to 17 nucleotides, 12 to 16 nucleotides, 12 to 15 nucleotides, 12 to 14 nucleotides, or 12 to 13 nucleotides. The extension primer can be extended, for example, 13 to 20 nucleotides, 13 to 19 nucleotides, 13 to 18 nucleotides, 13 to 17 nucleotides, 13 to 16 nucleotides, 13 to 15 nucleotides, or 13 to 14 nucleotides. The extension primer can be extended, for example, 14 to 20 nucleotides, 14 to 19 nucleotides, 14 to 18 nucleotides, 14 to 17 nucleotides, 14 to 16 nucleotides, 14 to 15 nucleotides, 15 to 20 nucleotides, 15 to 19 nucleotides, 15 to 18 nucleotides, 15 to 17 nucleotides, 15 to 16 nucleotides, 16 to 20 nucleotides, 16 to 19 nucleotides, 16 to 18 nucleotides, 16 to 17 nucleotides, 17 to 20 nucleotides, 17 to 19 nucleotides, 17 to 18 nucleotides, 18 to 20 nucleotides, 18 to 19 nucleotides, or 19 to 20 nucleotides. The extension primer can be extended, for example, 20 nucleotides, 19 nucleotides, 18 nucleotides, 17 nucleotides, 16 nucleotides, 15 nucleotides, 14 nucleotides, 13 nucleotides, 12 nucleotides, 11 nucleotides, 10 nucleotides, 9 nucleotides, 8 nucleotides, 7 nucleotides, 6 nucleotides, 5 nucleotides, 4 nucleotides, or 3 nucleotides.

3. Extension Primer Sets

Multiple extension primers can be used together, such as in sets of more than one extension primer. Primers in such sets can have any or all of a variety of characteristics in common, that differ, or a combination. Thus, for example, a set of extension primers can be made up of extension primers targeted to different target templates of nucleic acid sequences, extension primers having the same or similar hybrid stability, extension primers having the same or similar melting temperature, extension primers having a common non-target complement portion, extension primers to be extended by the same number of nucleotides, extension primers that when extended form extended extension primers having the same or similar hybrid stability, extension primers that when extended form extended extension primers having the same or similar melting temperature, or a combination of two or more of such features. Any of the disclosed characteristics of extension primers can be the subject of similarity or difference between extension primers in a set; all such sets are specifically contemplated and should be considered disclosed.

Where multiple extension primers are used, or for any set of extension primers, the melting temperature of the extension primers can be referred to in different ways. For example, the extension primers can each have a given melting temperature or range of melting temperature; one or more, two or more, three or more, etc., of the extension primers can have a given melting temperature or range of melting temperature; or at least one, at least two, at least three, etc., of the extension primers can have a given melting temperature or range of melting temperature. All of the extension primer melting temperatures, melting temperature ranges, and melting temperature relationships disclosed herein can be embodied in groups and sets of extension primers where the melting temperatures are referred to in the manner described above. Such concepts are specifically contemplated and should be considered disclosed herein. Thus, for example, the extension primer melting temperature range of about 10° C. to about 16° C. lower than the temperature at which the extension primers and target templates are incubated (one of the ranges described above) can be combined with the above concepts to result in a group or set of extension primers in which, for example, the extension primers can each have a melting temperature of about 10° C. to about 16° C. lower than the temperature at which the extension primers and target templates are incubated; one or more, two or more, three or more, etc., of the extension primers can have a melting temperature of about 10° C. to about 16° C. lower than the temperature at which the extension primers and target templates are incubated; or at least one, at least two, at least three, etc., of the extension primers can have a melting temperature of about 10° C. to about 16° C. lower than the temperature at which the extension primers and target templates are incubated. All such combinations are contemplated and should be considered specifically disclosed.

Matched extension primers are a useful form of extension primer set (and are a useful component of extension primer sets). Matched extension primers are two or more extension primers that are targeted to different forms of a nucleic acid sequence of interest. For example, one of the matched extension primers can be targeted to a wild type sequence and another matched extension primer can be targeted to a mutant or modified sequence. By using both extension primers in a given assay, a signal will be obtained whether or not the mutant sequence is present. That is, if the wild type sequence is present, the wild type extension primer will be extended and detected, if the mutant sequence is present, the mutant extension primer will be extended and detected, and if both sequences are present, both extension primers will be extended and detected. In this way, both a “positive” result (detection of the mutant sequence) and a “negative” result (no detection of the mutant sequence) will give an extension result, thus indicating the reliability of any negative result. As many matched extension primers can be used as there are alternative sequences possible or expected for a given nucleic acid sequence. Multiple sets of matched extension primers can be present in an extension primer set (a set of matched extension primers would thus be a subset of the main set of extension primers).

A useful form of extension primer set is a set where all or a subset of the extension primers have the same or similar hybrid stability or the same or similar melting temperature. Such similarity gives the extension primers similar thermodynamic behavior and can give the disclosed method more consistent operation when using multiple extension primers. The hybrid stability and/or melting temperature of extension primers can be calculated using known formulas and principles of thermodynamics (see, for example, Santa Lucia et al., Biochemistry 35:3555-3562 (1996); Freier et al., Proc. Natl. Acad. Sci. USA 83:9373-9377 (1986); Breslauer et al., Proc. Natl. Acad. Sci. USA 83:3746-3750 (1986)). The hybrid stability of the extension primers can be made more similar (a process that can be referred to as smoothing the hybrid stabilities) by, for example, chemically modifying the extension primers (Nguyen et al., Nucleic Acids Res. 25(15):3059-3065 (1997); Hohsisel, Nucleic Acids Res. 24(3):430-432 (1996)). Hybrid stability can also be smoothed by carrying out the hybridization under specialized conditions (Nguyen et al., Nucleic Acids Res. 27(6):1492-1498 (1999); Wood et al., Proc. Natl. Acad. Sci. USA 82(6):1585-1588 (1985)).

Another particularly useful means of smoothing hybrid stability or melting temperature of the extension primers is to vary the length of the extension primers (or the target complement portion of the extension primers). This would allow adjustment of the hybrid stability of each extension primer so that all of the extension primers had similar hybrid stabilities (to the extent possible). Since the addition or deletion of a single nucleotide from an extension primer will change the hybrid stability of the extension primer by a fixed increment, it is understood that the hybrid stabilities of the extension primers typically will not be equal. For this reason, smoothing of hybrid stability of extension primers as used herein refers to any increase in the similarity of the hybrid stabilities of extension primers (or, put another way, any reduction in the differences in hybrid stabilities of the extension primers).

Similarity of hybrid stability of a set or group of extension primers can be referred to by the percent difference in stability of the extension primers in the set that form the most stable and least stable hybrids. Thus, a set of extension primers can have, for example, about 1%, about 2%, about 3%, about 4%, about 5%, about 6%, about 7%, about 8%, about 9%, about 10%, greater than about 10%, 1%, 2%, 3%, 4%, 5%, 6%, 7%, 8%, 9%, 10%, or greater than 10% difference in hybrid stability. Similarity of melting temperature of a set or group of extension primers can be referred to by the range of melting temperatures of the extension primers in the set. Thus, a set of extension primers can have, for example, extension primers with melting temperatures within about 10° C., about 9° C., about 8° C., about 7° C., about 6° C., about 5° C., about 4° C., about 3° C., about 2° C., about 1° C., 10° C., 9° C., 8° C., 7° C., 6° C., 5° C., 4° C., 3° C., 2° C., or 1° C.

The efficiency of the disclosed method can also be improved by grouping extension primers of similar hybrid stability in different assays, reactions, and/or reaction chambers that can be subjected to different reaction conditions. In this way, the reaction conditions can be optimized for particular classes of extension primers.

Extension primers can also be designed to produce extended extension primers that have the same or similar hybrid stability or the same or similar melting temperature. Particularly useful are sets of extension primers where the extension primers have the same or similar hybrid stability or the same or similar melting temperature and that, when extended, result in extended extension primers having the same or similar hybrid stability or the same or similar melting temperature.

B. Target Template

Target templates serve as templates for extension of extension primers. Target templates can be any molecule that can interact with extension primers in a nucleotide sequence-specific manner and which can serve as template for extension of the extension primers. Target templates can interact with extension primers and extended extension primers. Target templates can interact with the target complement portion of a corresponding extension primer and with the extended target complement portion of a corresponding extended extension primer. Target templates include a primer complement region, an extension region, and, generally, a replication terminating feature. The primer complement region can interact with the target complement portion of a corresponding extension primer and with the target complement portion of the extended target complement portion of a corresponding extended extension primer. The extension region of target templates serves as template for, and can interact with, the extension portion of a corresponding extended extension primer. The target complement region and the extension region of a target template together constitute an extended primer complement region.

Useful target templates are, for example, nucleic acid molecules, oligonucleotides and oligonucleotide analogs. Target templates can be, or can be derived from, for example, a source nucleic acid, source nucleic acid sequence, and/or source nucleic acid sample. Thus, for example, nucleic acids in a nucleic acid sample, processed forms of such nucleic acids, and synthetic oligonucleotides can be used as target templates.

Target templates generally correspond to one or more extension primers and one or more extended extension primers. Where there are different target templates, different extension primers, and/or different extended extension primers, the nucleotide sequence-specific interaction generally occurs only between particular target templates and particular extension primers and extended extension primers. That is, target templates can be specific to particular extension primers and extended extension primers based on the specificity of the nucleotide sequence-specific interaction. Target templates having such a relationship with a given extension primer and/or extended extension primer can be said to correspond to the extension primer and/or extended extension primer. Similarly, the extension primers and extended extension primers that have such a relationship with a given target template can be said to correspond to the extended extension primers. Where target templates are, or are derived from, a source nucleic acid, source nucleic acid sequence, and/or source nucleic acid sample, the target templates can be said to correspond to the source nucleic acid, source nucleic acid sequence, and/or source nucleic acid sample. Similarly, such source nucleic acids, source nucleic acid sequences, and source nucleic acid samples can be said to correspond to target templates which are, or are derived from, the source nucleic acids, source nucleic acid sequences, and source nucleic acid samples.

In general, the target template causes termination of primer extension by having a replication terminating feature. The replication terminating feature is something (such as an abasic nucleotide, a 5′ end, or a derivatized nucleotide) that prevents further extension of the primer. It should be noted that replication terminated by a feature of the template is distinct from termination caused by a feature of the extending strand such as a dideoxynucleotide. In the present method where replication terminating features are part of the target template termination of extension is determined by the template. Target templates can have multiple replication terminating features. Where there are multiple replication terminating features, it is generally the replication terminating feature 3′ of and closest to the primer complement region (or, put another way, the most 5′ replication termination feature in the extension region of the target template) that determines termination of primer extension.

Target templates can be separate molecules, can be a part of a larger molecules, and/or can be present in the same molecules. That is, for example, a nucleic acid molecule can include more than one target template. As discussed elsewhere herein, target templates can be defined by the extension primers which target them. Thus, for example, multiple target templates can be on the same nucleic acid molecule if multiple sequences in a nucleic acid molecule are targeted by different extension primers. Generally, target templates that are not at the 5′ end of a nucleic acid molecule will include a non-5′ end replication terminating feature.

Where the replication terminating feature is a 5′ end, the replication terminating feature can be, for example, the normal, natural, or naturally occurring end of a nucleic acid molecule, or it can be generated. Any treatment that results in cleavage or digestion of nucleic acid can be sued to generate a 5′ end. Many such treatments are known. For example, numerous endonucleases and exonucleases are known and can be used. Useful nucleases include endonucleases that cleave at defined location based on the nucleotide sequence at or adjacent to the cleavage site. A similar effect (sequence-specific cleavage) can be achieved using sequence-specific probes that mediate nucleic acid cleavage. 5′ ends can also be generated by, for example, damaging or modifying nucleotides (thus creating a labile or otherwise susceptible site) followed by cleavage or scission of the nucleic acid molecule at that site. For example, 5′ ends can be generated by incorporating deoxyuridine into nucleic acids, removing the uracil base portion to create an abasic nucleotide, and breaking the strand at the abasic site (using, for example, T4 endonuclease V (Epicentre Technologies, USA)). The base portion can be removed, for example, using uracil DNA glycosylase (Epicentre Technologies, USA). Treatment of abasic nucleotide residues with T4 endonuclease V cleaves the cDNA fragments at the site of the abasic nucleotide residue. The site of modification can be random or directed depending on the technique involved. 5′ ends can also be generated by ionizing radiation.

Non-5′ end replication terminating features can be any nucleotide, modified nucleotide, or extra component in a nucleic acid molecule that prevents or terminates replication. Replication need not terminate at the site of the replication terminating feature; the replication terminating feature need only cause or facilitate termination. Replication terminating features can include abasic and derivatized nucleotides. Abasic nucleotides can be incorporated into nucleic acids, added to nucleic acids, or created in nucleic acids by removal of the base portion of a nucleotide. For example, abasic nucleotides can be generated by incorporating deoxyuridine into nucleic acids and removing the uracil base portion to create an abasic nucleotide. The base portion can be removed, for example, using uracil DNA glycosylase (Epicentre Technologies, USA).

C. Extended Extension Primers

Extended extension primers are the product of extension of extension primers using a target template as the template. After extension, extended extension primers dissociate from target templates, allowing other extension primer to interact with the target template and be extended. Extended extension primers can also interact with detection probes and/or anchor probes. The extended extension primers can interact with target templates, detection probes, and anchor probes in a nucleotide sequence-specific manner (by base pairing or hybridization, for example). Where there are different extended extension primers, different target templates, different detection probes, and/or different anchor probes, the nucleotide sequence-specific interaction generally occurs only between particular extended extension primers and particular target templates, detection probes, and/or anchor primers. That is, extended extension primers can be specific to particular target templates, detection probes, and/or anchor primers based on the specificity of the nucleotide sequence-specific interaction. Extended extension primers having such a relationship with a given target template, detection probe, and/or anchor probe can be said to correspond to the target template, detection probe, and/or anchor probe. Similarly, the target templates, detection probes, and/or anchor probes that have such a relationship with a given extended extension primer can be said to correspond to the extended extension primers.

Useful extended extension primers comprise oligonucleotides, oligonucleotide analogs, or a combination. Extended extension primers can comprise an extended target complement portion and, optionally, a non-target complement portion. The extended target complement portion is complementary to sequence in a target template referred to as the extended primer complement region of the target template. The extended template complement portion of an extended extension primer comprises a target complement portion and an extension portion. The target complement portion is the target complement portion of the extension primer used to form the extended extension primer. The extension portion comprises the nucleotides added to the extension primer by extension. Thus, an extended extension primer can also be said to comprise an extension portion, a target complement portion, and, optionally, a non-target complement portion.

Once an extension primer is extended it is an extended extension primer (that is, the extension primer with the nucleotides added by the extension of the primer). Both the extension primer and the extended extension primer are complementary to the target template, with the extended extension primer having more complementary nucleotides.

Extension primers will be extended from their location of interaction with the target template to a replication terminating feature in the target template. The length of this extension (and thus the length of the extension portion of the extended extension primer) is determined by the location of interaction and the location of the replication terminating feature. Extension primers can be extended in the disclosed method by any number of nucleotides that allows the cycle of interaction, extension, and dissociation to take place. Generally, this feature is related to the relative melting temperatures of the extension primer and of the extended extension primer that results. The extension primer that is extended to form a given extended extension primer can be said to correspond to the extended extension primer. Similarly, the extended extension primer that results from extension of a given extension primer can be said to correspond to the extension primer.

Design of extended extension primers generally should take into account the relative melting temperatures of the extension primer and of the extended extension primer that results. Thus, the length of the extension portion of the extended extension primer generally can be adjusted in design based on the composition of the extension portion and the length and composition of the target complement portion of the extension primer. The length of the extension portion (that is, the length of extension) is determined by the distance between the 3′ end of the extension primer when interacting with the target template and this distance can be adjusted during design by locating the 3′ end of the extension primer appropriately. It should be understood that, if desired, target templates can be designed to work with particular extension primers. Thus, the length and composition of the primer complement region of target templates can be adjusted based on the relative melting temperatures of the extension primer and of the extended extension primer that results.

Design of extended extension primers should also take into account the location of replication terminating features. That is, the primer complement region of the target templates can be chosen based on the location of replication terminating features. Alternatively, the location of replication terminating features can be introduced at particular locations in nucleic acids (and the extended extension primers designed based on these locations). The distance between the primer complement region of the target template and the replication terminating feature generally determines the number of nucleotides added during extension and thus affects the melting temperature of the extended extension primer.

The extended target complement portion of an extended extension primer is based on the particular sequences—in a nucleic acid sample, for example—to which the source extension primer is targeted. Design of targeting of extension primers is described elsewhere herein.

Extended extension primers can contain additional sequence (and/or other features) that is not complementary to any part of the target template. This sequence is referred to as the non-target complement portion of the extended extension primer. The non-target complement portion of extended extension primers is the non-target complement portion of the extension primer used to form the extended extension primer. The non-target complement portion of the extended extension primer, if present, can facilitate, for example, detection, immobilization, or separation of the primers. The non-target complement portion of an extended extension primer may be any length, but is generally 1 to 100 nucleotides long, and preferably 4 to 8 nucleotides long. The non-target complement portion is generally at the 5′ end of the extended extension primer. To aid in detection and quantitation of extended extension primers, extended extension primers can include one or more detection labels. Extended extension primers can, but need not, include a non-target complement portion and/or features other than the extended target complement portion. Thus, for example, extended extension primers may consist of an extended target complement portion, extended extension primers may comprise an extended target complement portion, and extended extension primers may comprise nucleotides where the nucleotides consist of an extended target complement portion.

Extended extension primers can have one or more modified nucleotides. Such extended extension primers are referred to herein as modified extended extension primers. Some forms of modified extended extension primers, such as RNA/2′-O-methyl RNA chimeric extended extension primers, have a higher melting temperature than DNA primers. Also, extended extension primers made of RNA will be exonuclease resistant. These and other nucleotide modifications are described more extensively elsewhere herein.

1. Extended Extension Primer Melting Temperature

The melting temperatures (Tₘ) of extended extension primers are related to the dissociation constants (k_{d}) for the extended extension primers, and the melting temperature and dissociation constant are related to the hybrid stability of extended extension primers. The same relationships exist for extension primers. The relationship between these characteristics in the extended extension primers and these characteristics in their cognate extension primers can affect the amplification reaction, and thus can be a factor in the design of extended extension primers. As the Tₘ of the extended extension primer increases relative to the Tₘ of the extension primer, the dissociation rate of the extended extension primer decreases relative to the dissociation rate of the extension primer. This relative decrease will generally increase the ratio of extended extension primer/target template (that is, extended extension primer bound to target template) to extension primer/target template (that is, the extension primer bound to target template) for a given concentration of extended extension primer and extension primer. Because the amplification reaction proceeds more efficiently, in general, when the ratio of extended extension primer/target template to extension primer/target template is lower, it is desirable to limit the difference in melting temperature of the extended extension primer and the extension primer. It should be understood that differences in the concentration of the extension primer and extended extension primers will also affect the ratio of extended extension primer/target template to extension primer/target template, with higher extension primer concentrations and lower extended extension primer concentrations decreasing the ratio as discuss more fully elsewhere herein. This effect can be used to counteract some of the effect of the difference in melting temperature of the extension primer and extended extension primer.

Two different interactions of extended extension primers are most significant to the disclosed method: the interaction of extended extension primers with target templates and the interaction of extended extension primer with detection probes. As a result, the melting temperature of extended extension primers when interacting with target templates and the melting temperature of extended extension primers when interacting with detection probes are most significant to the disclosed method. Unless the context clearly indicates otherwise, reference herein to the melting temperature of an extended extension primer refers to the melting temperature of the extended extension primer when interacting with a target template. Because the extended target complement portion of an extended extension primer interacts with the target template, and because extended extension primers can include portions and features other than an extended target complement portion, it should be understood that reference to the melting temperature of an extended extension primer refers to the melting temperature of the extended target complement portion of the extended extension primer when interacting with the target template. Similarly, because the extended primer complement region of a target template interacts with the extended extension primer, and because target templates can include portions, regions, and features other than an extended primer complement region, it should be understood that reference to the melting temperature of an extended extension primer refers to the melting temperature of the extended extension primer when interacting with the extended primer complement region of the target template. It also should be understood that reference to the melting temperature of an extended extension primer refers to the melting temperature of the extended target complement of the extension primer when interacting with the extended primer complement region of the target template. The interaction of an extension primer and a target template can be described in any or all of these ways and should be understood to encompass all of these descriptions.

When the melting temperature of an extended extension primer interacting with a detection probe is intended, interaction with the detection probe will be specified unless the context makes it clear that such interaction is intended. The melting temperature of an extended extension primer when interacting with a sequence or component other than a target template or detection probe is sometimes relevant and may be referred to explicitly herein. For example, the melting temperature of an extended extension primer when interacting with an anchor probe may be referred to explicitly as such.

The melting temperature of extended extension primers and other disclosed components and oligonucleotides can be calculated using known formulas and principles of thermodynamics (see, for example, Santa Lucia et al., Biochemistry 35:3555-3562 (1996); Freier et al., Proc. Natl. Acad. Sci. USA 83:9373-9377 (1986); Breslauer et al., Proc. Natl. Acad. Sci. USA 83:3746-3750 (1986)). These principles are described more fully elsewhere herein. Melting temperatures can also be determined empirically by measuring the relevant interaction at different temperatures. Where the melting temperatures of extended extension primers and other components are to be compared, the melting temperatures can be determined using the same technique or calculation.

i. Difference in Melting Temperature

For extended extension primer design purposes, the difference in melting temperature can be expressed as the percent difference in melting temperature of the extended extension primer and the extension primer. Examples of such percent differences in melting temperature are described elsewhere herein. The melting temperature of extended extension primers can also be expressed in terms of the difference in the melting temperature of their cognate extension primer. Examples of such differences in melting temperature are described elsewhere herein.

The melting temperature of extended extension primers can also be expressed in terms of the difference in the melting temperature when interacting with a detection probe and the melting temperature when interacting with the target template. Although the following description refers to the melting temperature of extended extension primers when interacting with detection probes in relation to the melting temperature of extended extension primers when interacting with target templates, this description should also be understood to disclose and describe the melting temperature of the extended target complement portion of extended extension primers when interacting with target templates in relation to the melting temperature of extended extension primers when interacting with detection probes, the melting temperature of extended extension primers when interacting with target templates in relation to the melting temperature of the extended extension primers when interacting with the primer complement portion of detection probes, the melting temperature of the extended target complement portion of extended extension primers when interacting with target templates in relation to the melting temperature of the extended extension primers when interacting with the primer complement portion of detection probes, the melting temperature of extended extension primers when interacting with target templates to which the extended extension primers correspond in relation to the melting temperature of extended extension primers when interacting with detection probes, the melting temperature of extended extension primers when interacting with target templates in relation to the melting temperature of extended extension primers when interacting with detection probes to which the extended extension primers correspond, and the melting temperature of extended extension primers when interacting with target templates to which the extended extension primers correspond in relation to the melting temperature of extended extension primers when interacting with detection probes to which the extended extension primers correspond.

The melting temperature of extended extension primers when interacting with target templates can be, for example, about 2° C. to about 25° C., about 3° C. to about 25° C., about 4° C. to about 25° C., about 5° C. to about 25° C., about 6° C. to about 25° C., about 7° C. to about 25° C., about 8° C. to about 25° C., about 9° C. to about 25° C., about 10° C. to about 25° C., about 11° C. to about 25° C., about 12° C. to about 25° C., about 13° C. to about 25° C., about 14° C. to about 25° C., about 15° C. to about 25° C., about 16° C. to about 25° C., about 17° C. to about 25° C., about 18° C. to about 25° C., about 19° C. to about 25° C., about 20° C. to about 25° C., about 21° C. to about 25° C., about 22° C. to about 25° C., about 23° C. to about 25° C., or about 24° C. to about 25° C. lower than the melting temperature of the extended extension primer when interacting with detection probes. The melting temperature of extended extension primers when interacting with target templates can be, for example, about 2° C. to about 24° C., about 3° C. to about 24° C., about 4° C. to about 24° C., about 5° C. to about 24° C., about 6° C. to about 24° C., about 7° C. to about 24° C., about 8° C. to about 24° C., about 9° C. to about 24° C., about 10° C. to about 24° C., about 11° C. to about 24° C., about 12° C. to about 24° C., about 13° C. to about 24° C., about 14° C. to about 24° C., about 15° C. to about 24° C., about 16° C. to about 24° C., about 17° C. to about 24° C., about 18° C. to about 24° C., about 19° C. to about 24° C., about 20° C. to about 24° C., about 21° C. to about 24° C., about 22° C. to about 25° C., or about 23° C. to about 24° C. lower than the melting temperature of the extended extension primer when interacting with detection probes.

The melting temperature of extended extension primers when interacting with target templates can be, for example, about 2° C. to about 23° C., about 3° C. to about 23° C., about 4° C. to about 23° C., about 5° C. to about 23° C., about 6° C. to about 23° C., about 7° C. to about 23° C., about 8° C. to about 23° C., about 9° C. to about 23° C., about 10° C. to about 23° C., about 11° C. to about 23° C., about 12° C. to about 23° C., about 13° C. to about 23° C. about 14° C. to about 23° C., about 15° C. to about 23° C., about 16° C. to about 23° C., about 17° C. to about 23° C., about 18° C. to about 23° C., about 19° C. to about 23° C., about 20° C. to about 23° C., about 21° C. to about 23° C., or about 22° C. to about 23° C. lower than the melting temperature of the extended extension primer when interacting with detection probes. The melting temperature of extended extension primers when interacting with target templates can be, for example, about 2° C. to about 22° C., about 3° C. to about 22° C., about 4° C. to about 22° C., about 5° C. to about 22° C., about 6° C. to about 22° C., about 7° C. to about 22° C., about 8° C. to about 22° C., about 9° C. to about 22° C., about 10° C. to about 22° C., about 11° C. to about 22° C., about 12° C. to about 22° C., about 13° C. to about 22° C., about 14° C. to about 22° C. about 15° C. to about 22° C., about 16° C. to about 22° C., about 17° C. to about 22° C., or about 18° C. to about 22° C., about 19° C. to about 22° C., about 20° C. to about 22° C., or about 21° C. to about 22° C. lower than the melting temperature of the extended extension primer when interacting with detection probes.

The melting temperature of extended extension primers when interacting with target templates can be, for example, about 2° C. to about 21° C., about 3° C. to about 21° C., about 4° C. to about 21° C., about 5° C. to about 21° C., about 6° C. to about 21° C., about 7° C. to about 21° C., about 8° C. to about 21° C., about 9° C. to about 21° C., about 10° C. to about 21° C., about 11° C. to about 21° C., about 12° C. to about 21° C., about 13° C. to about 21° C., about 14° C. to about 21° C., about 15° C. to about 21° C., about 16° C. to about 21° C., about 17° C. to about 21° C., about 18° C. to about 21° C., about 19° C. to about 21° C., or about 20° C. to about 21° C. lower than the melting temperature of the extended extension primer when interacting with detection probes. The melting temperature of extended extension primers when interacting with target templates can be, for example, about 2° C. to about 20° C., about 3° C. to about 20° C., about 4° C. to about 20° C., about 5° C. to about 20° C., about 6° C. to about 20° C., about 7° C. to about 20° C., about 8° C. to about 20° C., about 9° C. to about 20° C., about 10° C. to about 20° C., about 11° C. to about 20° C., about 12° C. to about 20° C., about 13° C. to about 20° C., about 14° C. to about 20° C., about 15° C. to about 20° C., about 16° C. to about 20° C., about 17° C. to about 20° C., about 18° C. to about 20° C., or about 19° C. to about 20° C. lower than the melting temperature of the extended extension primer when interacting with detection probes.

The melting temperature of extended extension primers when interacting with target templates can be, for example, about 2° C. to about 19° C., about 3° C. to about 19° C., about 4° C. to about 19° C., about 5° C. to about 19° C., about 6° C. to about 19° C., about 7° C. to about 19° C., about 8° C. to about 19° C., about 9° C. to about 19° C., about 10° C. to about 19° C., about 11° C. to about 19° C., about 12° C. to about 19° C., about 13° C. to about 19° C., 14° C. to about 19° C., about 15° C. to about 19° C., about 16° C. to about 19° C., about 17° C. to about 19° C., or about 18° C. to about 19° C. lower than the melting temperature of the extended extension primer when interacting with detection probes. The melting temperature of extended extension primers when interacting with target templates can be, for example, about 2° C. to about 18° C., about 3° C. to about 18° C., about 4° C. to about 18° C., about 5° C. to about 18° C., about 6° C. to about 18° C., about 7° C. to about 18° C., about 8° C. to about 18° C., about 9° C. to about 18° C., about 10° C. to about 18° C., about 11° C. to about 18° C., about 12° C. to about 18° C., about 13° C. to about 18° C., about 14° C. to about 18° C., about 15° C. to about 18° C., about 16° C. to about 18° C., or about 17° C. to about 18° C. lower than the melting temperature of the extended extension primer when interacting with detection probes.

The melting temperature of extended extension primers when interacting with target templates can be, for example, about 2° C. to about 17° C., about 3° C. to about 17° C., about 4° C. to about 17° C., about 5° C. to about 17° C., about 6° C. to about 17° C., about 7° C. to about 17° C., about 8° C. to about 17° C., about 9° C. to about 17° C., about 10° C. to about 17° C., about 11° C. to about 17° C., about 12° C. to about 17° C., about 13° C. to about 17° C., about 14° C. to about 17° C., about 15° C. to about 17° C., or about 16° C. to about 17° C. lower than the melting temperature of the extended extension primer when interacting with detection probes. The melting temperature of extended extension primers when interacting with target templates can be, for example, about 2° C. to about 16° C., about 3° C. to about 16° C., about 4° C. to about 16° C., about 5° C. to about 16° C., about 6° C. to about 16° C., about 7° C. to about 16° C., about 8° C. to about 16° C., about 9° C. to about 16° C., about 10° C. to about 16° C., about 11° C. to about 16° C., about 12° C. to about 16° C., about 13° C. to about 16° C., about 14° C. to about 16° C., or about 15° C. to about 16° C. lower than the melting temperature of the extended extension primer when interacting with detection probes.

The melting temperature of extended extension primers when interacting with target templates can be, for example, about 2° C. to about 15° C., about 3° C. to about 15° C., about 4° C. to about 15° C., about 5° C. to about 15° C., about 6° C. to about 15° C., about 7° C. to about 15° C., about 8° C. to about 15° C., about 9° C. to about 15° C., about 10° C. to about 15° C., about 11° C. to about 15° C., about 12° C. to about 15° C., about 13° C. to about 15° C., to about 14° C. to about 15° C. lower than the melting temperature of the extended extension primer when interacting with detection probes. The melting temperature of extended extension primers when interacting with target templates can be, for example, about 2° C. to about 14° C., about 3° C. to about 14° C., about 4° C. to about 14° C., about 5° C. to about 14° C., about 6° C. to about 14° C., about 7° C. to about 14° C., about 8° C. to about 14° C., about 9° C. to about 14° C., about 10° C. to about 14° C., about 11° C. to about 14° C., about 12° C. to about 14° C., or about 13° C. to about 14° C. lower than the melting temperature of the extended extension primer when interacting with detection probes. The melting temperature of extended extension primers when interacting with target templates can be, for example, about 2° C. to about 13° C., about 3° C. to about 13° C., about 4° C. to about 13° C., about 5° C. to about 13° C., about 6° C. to about 13° C., about 7° C. to about 13° C., about 8° C. to about 13° C., about 9° C. to about 13° C., about 10° C. to about 13° C., about 11° C. to about 13° C., or about 12° C. to about 13° C. lower than the melting temperature of the extended extension primer when interacting with detection probes.

The melting temperature of extended extension primers when interacting with target templates can be, for example, about 2° C. to about 12° C., about 3° C. to about 12° C., about 7° C. to about 4° C. to about 12° C., about 5° C. to about 12° C., about 6° C. to about 12° C., about 7° C. to about 12° C., about 8° C. to about 12° C., about 9° C. to about 12° C., about 10° C. to about 12° C., or about 11° C. to about 12° C. lower than the melting temperature of the extended extension primer when interacting with detection probes. The melting temperature of extended extension primers when interacting with target templates can be, for example, about 2° C. to about 11° C., about 3° C. to about 11° C., about 4° C. to about 11° C., about 5° C. to about 11° C., about 6° C. to about 11° C., about 7° C. to about 11° C., about 8° C. to about 11° C., about 9° C. to about 11° C., or about 10° C. to about 11° C. lower than the melting temperature of the extended extension primer when interacting with detection probes. The melting temperature of extended extension primers when interacting with target templates can be, for example, about 2° C. to about 10° C., about 3° C. to about 10° C., about 4° C. to about 10° C., about 5° C. to about 10° C., about 6° C. to about 10° C., about 7° C. to about 10° C., about 8° C. to about 10° C., or about 9° C. to about 10° C. lower than the melting temperature of the extended extension primer when interacting with detection probes.

The melting temperature of extended extension primers when interacting with target templates can be, for example, about 2° C. to about 9° C., about 3° C. to about 9° C., about 4° C. to about 9° C., about 5° C. to about 9° C., about 6° C. to about 9° C., about 7° C. to about 9° C., or about 8° C. to about 9° C. lower than the melting temperature of the extended extension primer when interacting with detection probes. The melting temperature of extended extension primers when interacting with target templates can be, for example, about 2° C. to about 8° C., about 3° C. to about 8° C., about 4° C. to about 8° C., about 5° C. to about 8° C., about 6° C. to about 8° C., or about 7° C. to about 8° C. lower than the melting temperature of the extended extension primer when interacting with detection probes. The melting temperature of extended extension primers when interacting with target templates can be, for example, about 2° C. to about 7° C., about 3° C. to about 7° C., about 4° C. to about 7° C., about 5° C. to about 7° C., or about 6° C. to about 7° C. lower than the melting temperature of the extended extension primer when interacting with detection probes. The melting temperature of extended extension primers when interacting with target templates can be, for example, about 2° C. to about 6° C., about 3° C. to about 6° C., about 4° C. to about 6° C., about 5° C. to about 6° C., 2° C. to about 5° C., about 3° C. to about 5° C., about 4° C. to about 5° C., 2° C. to about 4° C., about 3° C. to about 4° C., or 2° C. to about 3° C. lower than the melting temperature of the extended extension primer when interacting with detection probes.

The melting temperature of extended extension primers when interacting with target templates can be, for example, about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., or about 25° C. lower than the melting temperature of the extended extension primer when interacting with detection probes. The melting temperature of extended extension primers when interacting with target templates can be, for example, 2° C., 3° C., 4° C., 5° C., 6° C., 7° C., 8° C., 9°C., 10° C., 11° C., 12° C., 13° C., 14° C., 15° C., 16° C., 17° C., 18° C., 19° C., 20° C., 21° C., 22° C., 23° C., 24° C., or 25° C. lower than the melting temperature of the extended extension primer when interacting with detection probes. All of the above relationships also can be described in terms of the melting temperature of the extended extension primer when interacting with detection probes being higher than the melting temperature of extended extension primers when interacting with target templates, and such alternative descriptions are contemplated and should be considered specifically disclosed.

Where the extended extension primer includes a non-target complement portion, the melting temperature of extended extension primers can also be expressed in terms of the difference in the melting temperature of the entire extended extension primer (that is, both the extended target complement portion and the non-target complement portion) and the melting temperature of the extended target complement portion of the extended extension primer. This distinction is generally relevant to the difference in the melting temperature of an extended extension primer to target templates (where only the target complement portion of the extended extension primer interacts with the target template) and the melting temperature of the extended extension primer to detection probes (when the entire extended extension primer can interact with the detection probe). Although the following description refers to the melting temperature of entire extended extension primers in relation to the melting temperature of the extended target complement portion of the extended extension primers, this description should also be understood to disclose and describe the melting temperature of the extended target complement portion of extended extension primers in relation to the melting temperature of entire extended extension primers, the melting temperature of the extended target complement portion of extended extension primers when interacting with target templates in relation to the melting temperature of entire extended extension primers when interacting with detection probes, the melting temperature of extended extension primers when interacting with target templates in relation to the melting temperature of the entire extended extension primers when interacting with the primer complement portion of detection probes, the melting temperature of the extended target complement portion of extended extension primers when interacting with target templates in relation to the melting temperature of the entire extended extension primers when interacting with the primer complement portion of detection probes, the melting temperature of extended extension primers when interacting with target templates to which the extended extension primers correspond in relation to the melting temperature of entire extended extension primers when interacting with detection probes, the melting temperature of extended extension primers when interacting with target templates in relation to the melting temperature of entire extended extension primers when interacting with detection probes to which the extended extension primers correspond, and the melting temperature of extended extension primers when interacting with target templates to which the extended extension primers correspond in relation to the melting temperature of entire extended extension primers when interacting with detection probes to which the extended extension primers correspond.

The melting temperature of the extended target complement portion of extended extension primers can be, for example, about 2° C. to about 25° C., about 3° C. to about 25° C., about 4° C. to about 25° C., about 5° C. to about 25° C., about 6° C. to about 25° C., about 7° C. to about 25° C., about 8° C. to about 25° C., about 9° C. to about 25° C., about 10° C. to about 25° C., 25° C., about 11° C. to about 25° C., about 12° C. to about 25° C., about 13° C. to about 25° C., about 14° C. to about 25° C., about 15° C. to about 25° C., about 16° C. to about 25° C., about 17° C. to about 25° C., about 18° C. to about 25° C., about 19° C. to about 25° C., about 20° C. to about 25° C., about 21° C. to about 25° C., about 22° C. to about 25° C., about 23° C. to about 25° C., or about 24° C. to about 25° C. lower than the melting temperature of the entire extended extension primer. The melting temperature of the extended target complement portion of extended extension primers can be, for example, about 2° C. to about 24° C., about 3° C. to about 24° C., about 4° C. to about 24° C., about 5° C. to about 24° C., about 6° C. to about 24° C., about 7° C. to about 24° C., about 8° C. to about 24° C., about 9° C. to about 24° C., about 10° C. to about 24° C., about 11° C. to about 24° C., about 12° C. to about 24° C., about 13° C. to about 24° C., about 14° C. to about 24° C., about 15° C. to about 24° C., about 16° C. to about 24° C., about 17° C. to about 24° C., about 18° C. to about 24° C., about 19° C. to about 24° C., about 20° C. to about 24° C., about 21° C. to about 24° C., about 22° C. to about 25° C., or about 23° C. to about 24° C. lower than the melting temperature of the entire extended extension primer.

The melting temperature of the extended target complement portion of extended extension primers can be, for example, about 2° C. to about 23° C., about 3° C. to about 23° C., about 4° C. to about 23° C., about 5° C. to about 23° C., about 6° C. to about 23° C., about 7° C. to about 23° C., about 8° C. to about 23° C., about 9° C. to about 23° C., about 10° C. to about 23° C., about 11° C. to about 23° C., about 12° C. to about 23° C., about 13° C. to about 23° C., about 14° C. to about 23° C., about 15° C. to about 23° C., about 16° C. to about 23° C., about 17° C. to about 23° C., about 18° C. to about 23° C., about 19° C. to about 23° C., about 20° C. to about 23° C., about 21° C. to about 23° C., or about 22° C. to about 23° C. lower than the melting temperature of the entire extended extension primer. The melting temperature of the extended target complement portion of extended extension primers can be, for example, about 2° C. to about 22° C., about 3° C. to about 22° C., about 4° C. to about 22° C., about 5° C. to about 22° C., about 6° C. to about 22° C., about 7° C. to about 22° C., about 8° C. to about 22° C., about 9° C. to about 22° C., about 10° C. to about 22° C., about 11° C. to about 22° C., about 12° C. to about 22° C., about 13° C. to about 22° C., about 14° C. to about 22° C., about 15° C. to about 22° C., about 16° C. to about 22° C., about 17° C. to about 22° C., about 18° C. to about 22° C., about 19° C. to about 22° C., about 20° C. to about 22° C., or about 21° C. to about 22° C. lower than the melting temperature of the entire extended extension primer.

The melting temperature of the extended target complement portion of extended extension primers can be, for example, about 2° C. to about 21° C., about 3° C. to about 21° C., about 4° C. to about 21° C., about 5° C. to about 21° C., about 6° C. to about 21° C., about 7° C. to about 21° C., about 8° C. to about 21° C., about 9° C. to about 21° C., about 10° C. to about 21° C., about 11° C. to about 21° C., about 12° C. to about 21° C., about 13° C. to about 21° C., about 14° C. to about 21° C., about 15° C. to about 21° C., about 16° C. to about 21° C., about 17° C. to about 21° C., about 18° C. to about 21° C., about 19° C. to about 21° C., or about 20° C. to about 21° C. lower than the melting temperature of the entire extended extension primer. The melting temperature of the extended target complement portion of extended extension primers can be, for example, about 2° C. to about 20° C., about 3° C. to about 20° C., about 4° C. to about 20° C., about 5° C. to about 20° C., about 6° C. to about 20° C., about 7° C. to about 20° C., about 8° C. to about 20° C., about 9° C. to about 20° C., about 10° C. to about 20° C., about 11° C. to about 20° C., about 12° C. to about 20° C., about 13° C. to about 20° C., about 14° C. to about 20° C., about 15° C. to about 20° C., about 16° C. to about 20° C., about 17° C. to 20° C., about 18° C. to about 20° C., or about 19° C. to about 20° C. lower than the melting temperature of the entire extended extension primer.

The melting temperature of the extended target complement portion of extended extension primers can be, for example, about 2° C. to about 19° C., about 3° C. to about 19° C., about 4° C. to about 19° C., about 5° C. to about 19° C., about 6° C. to about 19° C., about 7° C. to about 19° C., about 8° C. to about 19° C., about 9° C. to about 19° C., about 10° C. to about 19° C., about 11° C. to about 19° C., about 12° C. to about 19° C., about 13° C. to about 19° C., about 14° C. to about 19° C., about 15° C. to about 19° C., about 16° C. to about 19° C., 17° C. to about 19° C., or about 18° C. to about 19° C. lower than the melting temperature of the entire extended extension primer. The melting temperature of the extended target complement portion of extended extension primers can be, for example, about 2° C. to about 18° C., about 3° C. to about 18° C., about 4° C. to about 18° C., about 5° C. to about 18° C., about 6° C. to about 18° C., about 7° C. to about 18° C., about 8° C. to about 18° C., about 9° C. to about 18° C., about 10° C. to about 18° C., about 11° C. to about 18° C., about 12° C. to about 18° C., about 13° C. to about 18° C., about 14° C. to about 18° C., about 15° C. to about 18° C., about 16° C. to about 18° C., or about 17° C. to about 18° C. lower than the melting temperature of the entire extended extension primer.

The melting temperature of the extended target complement portion of extended extension primers can be, for example, about 2° C. to about 17° C., about 3° C. to about 17° C., about 4° C. to about 17° C., about 5° C. to about 17° C., about 6° C. to about 17° C., about 7° C. to about 17° C., about 8° C. to about 17° C., about 9° C. to about 17° C., about 10° C. to about 17° C., about 11° C. to about 17° C., about 12° C. to about 17° C., about 13° C. to about 17° C., about 14° C. to about 17° C., about 15° C. to about 17° C., or about 16° C. to about 17° C. lower than the melting temperature of the entire extended extension primer. The melting temperature of the extended target complement portion of extended extension primers can be, for example, about 2° C. to about 16° C., about 3° C. to about 16° C., about 4° C. to about 16° C., about 5° C. to about 16° C., about 6° C. to about 16° C., about 7° C. to about 16° C., about 8° C. to about 16° C., about 9° C. to about 16° C., about 10° C. to about 16° C., about 11° C. to about 16° C., about 12° C. to about 16° C., about 13° C. to about 16° C., about 14° C. to about 16° C., or about 15° C. to about 16° C. lower than the melting temperature of the entire extended extension primer.

The melting temperature of the extended target complement portion of extended extension primers can be, for example, about 2° C. to about 15° C., about 3° C. to about 15° C., about 4° C. to about 15° C., about 5° C. to about 15° C., about 6° C. to about 15° C., about 7° C. to about 15° C., about 8° C. to about 15° C., about 9° C. to about 15° C., about 10° C. to about 15° C., about 11° C. to about 15° C., about 12° C. to about 15° C., about 13° C. to about 15° C., or about 14° C. to about 15° C. lower than the melting temperature of the entire extended extension primer. The melting temperature of the extended target complement portion of extended extension primers can be, for example, about 2° C. to about 14° C., about 3° C. to about 14° C., about 4° C. to about 14° C., about 5° C. to about 14° C., about 6° C. to about 14° C., about 7° C. to about 14° C., about 8° C. to about 14° C., about 9° C. to about 14° C., about 10° C. to about 14° C., about 11° C. to about 14° C., about 12° C. to about 14° C., or about 13° C. to about 14° C. lower than the melting temperature of the entire extended extension primer. The melting temperature of the extended target complement portion of extended extension primers can be, for example, about 2° C. to about 13° C., about 3° C. to about 13° C., about 4° C. to about 13° C., about 5° C. to about 13° C., about 6° C. to about 13° C., about 7° C. to about 13° C., about 8° C. to about 13° C., about 9° C. to about 13° C., about 10° C. to about 13° C., about 11° C. to about 13° C., or about 12° C. to about 13° C. lower than the melting temperature of the entire extended extension primer.

The melting temperature of the extended target complement portion of extended extension primers can be, for example, about 2° C. to about 12° C., about 3° C. to about 12° C., about 4° C. to about 12° C., about 5° C. to about 12° C., about 6° C. to about 12° C., about 7° C. to about 12° C., about 8° C. to about 12° C., about 9° C. to about 12° C., about 10° C. to about 12° C., or about 11° C. to about 12° C. lower than the melting temperature of the entire extended extension primer. The melting temperature of the extended target complement portion of extended extension primers can be, for example, about 2° C. to about 11° C., about 3° C. to about 11° C., about 4° C. to about 11° C., about 5° C. to about 11° C., about 6° C. to about 11° C., about 7° C. to about 11° C., about 8° C. to about 11° C., about 9° C. to about 11° C., or about 10° C. to about 11° C. lower than the melting temperature of the entire extended extension primer. The melting temperature of the extended target complement portion of extended extension primers can be, for example, about 2° C. to about 10° C., about 3° C. to about 10° C., about 4° C. to about 10° C., about 5° C. to about 10° C., about 6° C. to about 10° C., about 7° C. to about 10° C., about 8° C. to about 10° C., or about 9° C. to about 10° C. lower than the melting temperature of the entire extended extension primer.

The melting temperature of the extended target complement portion of extended extension primers can be, for example, about 2° C. to about 9° C., about 3° C. to about 9° C., about 4° C. to about 9° C., about 5° C. to about 9° C., about 6° C. to about 9° C., about 7° C., about 9° C., or about 8° C. to about 9° C. lower than the melting temperature of the entire extended extension primer. The melting temperature of the extended target complement portion of extended extension primers can be, for example, about 2° C. to about 8° C., about 3° C. to about 8° C., about 4° C. to about 8° C., about 5° C. to about 8° C., about 6° C. to about 8° C., or about 7° C. to about 8° C. lower than the melting temperature of the entire extended extension primer. The melting temperature of the extended target complement portion of extended extension primers can be, for example, about 2° C. to about 7° C., about 3° C. to about 7° C., about 4° C. to about 7° C., about 5° C. to about 7° C., or about 6° C. to about 7° C. lower than the melting temperature of the entire extended extension primer. The melting temperature of the extended target complement portion of extended extension primers can be, for example, about 2° C. to about 6° C., about 3° C. to about 6° C., about 4° C. to about 6° C., about 5° C. to about 6° C., 2° C. to about 5° C., about 3° C. to about 5° C., about 4° C. to about 5° C., 2° C. to about 4° C., about 3° C. to about 4° C., or 2° C. to about 3° C. lower than the melting temperature of the entire extended extension primer.

The melting temperature of the extended target complement portion of extended extension primers can be, for example, about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., or about 25° C. lower than the melting temperature of the entire extended extension primer. The melting temperature of the extended target complement portion of extended extension primers can be, for example, 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C. a lower than the melting temperature of the entire extended extension primer. All of the above relationships also can be described in terms of the melting temperature of the entire extended extension primer being higher than the melting temperature of the extended target complement portion of extended extension primers, and such alternative descriptions are contemplated and should be considered specifically disclosed.

The melting temperature of extended extension primers can also be expressed in terms of the difference from the temperature at which the extension primers and target templates are incubated. Although the following description refers to the melting temperature of extended extension primers in relation to the temperature at which the extension primers and target templates are incubated, this description should also be understood to disclose and describe the melting temperature of the extended target complement portion of extended extension primers in relation to the temperature at which the extension primers and target templates are incubated, the melting temperature of entire extended extension primers in relation to the temperature at which the extension primers and target templates are incubated, the melting temperature of extended extension primers when interacting with target templates in relation to the temperature at which the extension primers and target templates are incubated, and the melting temperature of extended extension primers when interacting with target templates to which the extended extension primers correspond in relation to the temperature at which the extension primers and target templates are incubated. The melting temperature of the extended extension primer can be, for example, about 0° C. to about 20° C., about 0° C. to about 19° C., about 0° C. to about 18° C., about 0° C. to about 17° C., about 0° C to about 16° C., about 0°to about 15° C., about 0° C. to about 14° C., about 0° C. to about 13° C., about 0° C. to about 12° C., about 0° C. to about 11° C., about 0° C. to about 10° C., about 0° C. to about 9° C., about 0° C. to about 8° C., about 0° C. to about 7° C., about 0° C. to about 6° C., about 0° C. to about 5° C., about 0° C. to about 4° C., about 0° C. to about 3° C., about 0° C. to about 2° C., or about 0° C. to about 1° C. higher than the temperature at which the extension primers and target templates are incubated.

The melting temperature of the extended extension primer can be, for example, about 1° C. to about 20° C., about 1° C. to about 19° C., about 1° C. to about 18° C., about 1° C. to about 17° C., about 1° C. to about 16° C., about 1° C. to about 15° C., about 1° C. to about 14° C., about 1° C. to about 13° C., about 1° C. to about 12° C., about 1° C. to about 11° C., about 1° C. to about 10° C., about 1° C. to about 9° C., about 1° C. to about 8° C., about 1° C. to about 7° C., 1° C. to about 6° C., about 1° C. to about 5° C., about 1° C. to about 4° C., about 1° C. to about 3° C., or about 1° C. to about 2° C. higher than the temperature at which the extension primers and target templates are incubated. The melting temperature of the extended extension primer can be, for example, about 2° C. to about 20° C., about 2° C. to about 19° C., about 2° C. to about 18° C., about 2° C. to about 17° C., about 2° C. to about 16° C., about 2° C. to about 15° C., about 2° C. to about 14° C., about 2° C. to about 13° C., about 2° C. to about 12° C., about 2° C. to about 11° C., about 2° C. to about 10° C., about 2° C. to about 9° C., about 2° C. to about 8° C., about 2° C. to about 7° C., about 2° C. to about 6° C., about 2° C. to about 5° C., about 2° C. to about 4° C., or about 2° C. to about 3° C. higher than the temperature at which the extension primers and target templates are incubated.

The melting temperature of the extended extension primer can be, for example, about 3° C. to about 20° C., about 3° C. to about 19° C., about 3° C. to about 18° C., about 3° C. to about 17° C., about 3° C. to about 16° C., about 3° C. to about 15° C., about 3° C. to about 14° C., about 3° C. to about 13° C., about 3° C. to about 12° C., about 3° C. to about 11° C., about 3° C. to about 10° C., about 3° C. to about 9° C., about 3° C. to about 8° C., about 3° C. to about 7° C., about 3° C. to about 6° C., about 3° C. to about 5° C., or about 3° C. to about 4° C. higher than the temperature at which the extension primers and target templates are incubated. The melting temperature of the extended extension primer can be, for example, about 4° C. to about 20° C., about 4° C. to about 19° C., about 4° C. to about 18° C., about 4° C. to about 17° C., about 4° C. to about 16° C., about 4° C. to about 15° C., about 4° C. to about 14° C., about 4° C. to about 13° C., about 4° C. to about 12° C., about 4° C. to about 11° C., about 4° C. to about 10° C., about 4° C. to about 9° C., about 4° C. to about 8° C., about 4° C. to about 7° C., about 4° C. to about 6° C., or about 4° C. to about 5° C. higher than the temperature at which the extension primers and target templates are incubated.

The melting temperature of the extended extension primer can be, for example, about 5° C. to about 20° C., about 5° C. to about 19° C., about 5° C. to about 18° C., about 5° C. to about 17° C., about 5° C. to about 16° C., about 5° C. to about 15° C., about 5° C. to about 14° C., about 5° C. to about 13° C., about 5° C. to about 12° C., about 5° C. to about 11° C., about 5° C., or about 10° C., about 5° C. to about 9° C., about 5° C. to about 8° C., about 5° C. to about 7° C., or about 5° C. to about 6° C. higher than the temperature at which the extension primers and target templates are incubated.

The melting temperature of the extended extension primer can be, for example, about 6° C. to about 20° C., about 6° C. to about 19° C., about 6° C. to about 18° C., about 6° C. to about 17° C., about 6° C. to about 16° C., about 6° C. to about 15° C., about 6° C. to about 14° C., about 6° C. to about 13° C., about 6° C. to about 12° C., about 6° C. to about 11° C., about 6° C. to about 10° C., about 6° C. to about 9° C., about 6° C. to about 8° C., or about 6° C. to about 7° C. higher than the temperature at which the extension primers and target templates are incubated. The melting temperature of the extended extension primer can be, for example, about 7° C. to about 20° C., about 7° C. to about 19° C., about 7° C. to about 18° C., about 7° C. to about 17° C., about 7° C. to about 16° C., about 7° C. to about 15° C., about 7° C. to about 14° C., about 7° C. to about 13° C., about 7° C. to about 12° C., about 7° C. to about 11° C., about 7° C. to about 10° C., about 7° C. to about 9° C., or about 7° C. to about 8° C. higher than the temperature at which the extension primers and target templates are incubated.

The melting temperature of the extended extension primer can be, for example, about 8° C. to about 20° C., about 8° C. to about 19° C., about 8° C. to about 18° C., about 8° C. to about 17° C., about 8° C. to about 16° C., about 8° C. to about 15° C., about 8° C. to about 14° C., about 8° C. to about 13° C., about 8° C. to about 12° C., about 8° C. to about 11° C., about 8° C. to about 10° C., or about 8° C. to about 9° C. higher than the temperature at which the extension primers and target templates are incubated. The melting temperature of the extended extension primer can be, for example, about 9° C. to about 20° C., about 9° C. to about 19° C., about 9° C. to about 18° C., about 9° C. to about 17° C., about 9° C. to about 16° C., about 9° C., about 15° C., about 9° C. to about 14° C., about 9° C. to about 13° C., about 9° C. to about 12° C., about 9° C. to about 11° C., or about 9° C. to about 10° C. higher than the temperature at which the extension primers and target templates are incubated.

The melting temperature of the extended extension primer can be, for example, about 10° C. to about 20° C., about 10° C. to about 19° C., about 10° C. to about 18° C., about 10° C. to about 17° C., about 10° C. to about 16° C., about 10° C. to about 15° C., about 10° C. to about 14° C., about 10° C. to about 13° C., about 10° C. to about 12° C., or about 10° C. to about 11° C. higher than the temperature at which the extension primers and target templates are incubated. The melting temperature of the extended extension primer can be, for example, about 11° C. to about 20° C., about 11° C. to about 19° C., about 11° C. to about 18° C., about 11° C. to about 17° C., about 11° C. to about 16° C., about 11° C. to about 15° C., about 11° C. to about 14° C., about 11° C. to about 13° C., or about 11° C. to about 12° C. higher than the temperature at which the extension primers and target templates are incubated. The melting temperature of the extended extension primer can be, for example, about 12° C. to about 20° C., about 12° C. to about 19° C., about 12° C. to about 18° C., about 12° C. to about 17° C., about 12° C. to about 16° C., about 12° C. to about 15° C., about 12° C. to about 14° C., or about 12° C. to about 13° C. higher than the temperature at which the extension primers and target templates are incubated.

The melting temperature of the extended extension primer can be, for example, about 13° C. to about 20° C., about 13° C. to about 19° C., about 13° C. to about 18° C., about 13° C. to about 17° C., about 13° C. to about 16° C., about 13° C. to about 15° C., or about 13° C. to about 14° C. higher than the temperature at which the extension primers and target templates are incubated. The melting temperature of the extended extension primer can be, for example, about 14° C. to about 20° C., about 14° C. to about 19° C., about 14° C. to about 18° C., about 14° C. to about 17° C., about 14° C. to about 16° C., or about 14° C. to about 15° C. higher than the temperature at which the extension primers and target templates are incubated. The melting temperature of the extended extension primer can be, for example, about 15° C. to about 20° C., about 15° C. to about 19° C., about 15° C. to about 18° C., about 15° C. to about 17° C., or about 15° C. to about 16° C. higher than the temperature at which the extension primers and target templates are incubated.

The melting temperature of the extended extension primer can be, for example, about 16° C. to about 20° C., about 16° C. to about 19° C., about 16° C. to about 18° C., about 16° C. to about 17° C., 17° C. to about 20° C., about 17° C. to about 19° C., about 17° C. to about 18° C., about 18° C. to about 20° C., about 18° C. to about 19° C., or about 19° C. to about 20° C. higher than the temperature at which the extension primers and target templates are incubated. The melting temperature of the extended extension primer can be, for example, about 20° C., about 19° C., about 18° C., about 17° C., about 16° C., about 15° C., about 14° C., about 13° C., about 12° C., about 11° C., about 10° C., about 9° C., about 8° C., 7° C., about 6° C., about 5° C., about 4° C., about 3° C., about 2° C., about 1° C., about 0° C. higher than the temperature at which the extension primers and target templates are incubated. The melting temperature of the extended extension primer can be, for example, 20° C., 19° C., 18° C., 17° C., 16° C., 15° C., 14° C., 13° C., 12° C., 11° C., 10° C., 9° C., 8° C., 7° C., 6° C., 5° C. 4° C., 3° C., 2° C., 1° C., or 0° C. higher than the temperature at which the extension primers and target templates are incubated. All of the above relationships also can be described in terms of the temperature at which the extension primers and target templates are incubated being lower than the melting temperature of extended extension primers, and such alternative descriptions are contemplated and should be considered specifically disclosed.

ii. Melting Temperature

The melting temperature of extended extension primers can also be expressed in absolute terms. However, the principles discussed above and elsewhere herein, such as the temperature of incubation and its useful relationship to the melting temperature of extended extension primers, should be considered in designing the melting temperature of extended extension primers. Although the following description refers to the melting temperature of extended extension primers, this description should also be understood to disclose and describe the melting temperature of the extended target complement portion of extended extension primers, the melting temperature of extended extension primers when interacting with target templates, and the melting temperature of extended extension primers when interacting with target templates to which the extended extension primers correspond. The melting temperature of the extended extension primer can be, for example, about 64° C. to about 85° C., about 64° C. to about 83° C., about 64° C. to about 81° C., about 64° C. to about 79° C., about 64° C. to about 77° C., about 64° C. to about 76° C., about 64° C. to about 75° C., about 64° C. to about 74° C., about 64° C. to about 73° C., about 64° C. to about 72° C., about 64° C. to about 70° C., about 64° C. to about 68° C., or about 64° C. to about 66° C. The melting temperature of the extended extension primer can be, for example, about 66° C. to about 85° C., about 66° C. to about 83° C., about 66° C. to about 81° C., about 66° C. to about 79° C., about 66° C. to about 77° C., about 66° C. to about 76° C., about 66° C. to about 75° C., about 66° C. to about 74° C., about 66° C. to about 73° C., about 66° C. to about 72° C., about 66° C. to about 70° C., about 66° C. to about 68° C., or about 64° C. to about 66° C.

The melting temperature of the extended extension primer can be, for example, about 68° C. to about 85° C., about 68° C. to about 83° C., about 68° C. to about 81° C., about 68° C. to about 79° C., about 68° C. to about 77° C., about 68° C. to about 76° C., about 68° C. to about 75° C., about 68° C. to about 74° C., about 68° C. to about 73° C., about 68° C. to about 72° C., or about 68° C. to about 70° C. The melting temperature of the extended extension primer can be, for example, about 70° C. to about 85° C., about 70° C. to about 83° C., about 70° C. to about 81° C., about 70° C. to about 79° C., about 70° C. to about 77° C., about 70° C. to about 76° C., about 70° C. to about 75° C., about 70° C. to about 74° C., about 70° C. to about 73° C., or about 70° C. to about 72° C.

The melting temperature of the extended extension primer can be, for example, about 72° C. to about 85° C., about 72° C. to about 83° C., about 72° C. to about 81° C., about 72° C. to about 79° C., about 72° C. to about 77° C., about 72° C. to about 76° C., about 72° C. to about 75° C., about 72° C. to about 74° C., or about 72° C. to about 73° C. The melting temperature of the extended extension primer can be, for example, about 73° C. to about 85° C., about 73° C. to about 83° C., about 73° C. to about 81° C., about 73° C. to about 79° C., about 73° C. to about 77° C., about 73° C. to about 76° C., about 73° C. to about 75° C., or about 73° C. to about 74° C.

The melting temperature of the extended extension primer can be, for example, about 74° C. to about 85° C., about 74° C. to about 83° C., about 74° C. to about 81° C., about 74° C. to about 79° C., about 74° C. to about 77° C., about 74° C. to about 76° C., or about 74° C. to about 75° C. The melting temperature of the extended extension primer can be, for example, about 75° C. to about 85° C., about 75° C. to about 83° C., about 75° C. to about 81° C., about 75° C. to about 79° C., about 75° C. to about 77° C., or about 75° C. to about 76° C. to about temperature of the extended extension primer can be, for example, about 76° C. to about 85° C., about 76° C. to about 83° C., about 76° C. to about 81° C., about 76° C. to about 79° C., or about 76° C. to about 77° C.

The melting temperature of the extended extension primer can be, for example, about 77° C. to about 85° C., about 77° C. to about 83° C., about 77° C. to about 81° C., about 77° C. to about 79° C., about 74° C. to about 77° C., about 74° C. to about 76° C., or about 74° C. to about 75° C. The melting temperature of the extended extension primer can be, for example, about 79° C. to about 85° C., about 79° C. to about 83° C., about 79° C. to about 81° C., about 81° C. to about 85° C., about 81° C. to about 83° C., or about 83° C. to about 85° C. The melting temperature of the extended extension primer can be, for example, about 85° C., about 83° C., about 81° C., about 79° C., about 77° C., about 76° C., about 75° C., about 74° C., about 73° C., about 72° C., about 70° C., about 68° C., about 66° C., or about 64° C. The melting temperature of the extended extension primer can be, for example, 85° C., 83° C., 81° C., 79° C., 77° C., 76° C., 75° C., 74° C., 73° C., 72° C., 70° C., 68° C., 66° C. or 64° C.

2. Extension Portion

The design of extended extension primers can also be expressed in terms of the number of nucleotides added by extension. However, the effect of the added nucleotides on the melting temperature of the extended extension primer, and thus the effect on the relationship of the melting temperature of the extension primer and the extended extension primer, should be considered in designing extension primers. The length of extension (number of nucleotides added) corresponds to the length of the extension portion of extended extension primers and the length added to the target complement portion of extension primers to form the extended target complement portion of extension primers. The extended extension primer can have an extension portion of, for example, 3 to 20 nucleotides, 3 to 19 nucleotides, 3 to 18 nucleotides, 3 to 17 nucleotides, 3 to 16 nucleotides, 3 to 15 nucleotides, 3 to 14 nucleotides, 3 to 13 nucleotides, 3 to 12 nucleotides, 3 to 11 nucleotides, 3 to 10 nucleotides, 3 to 9 nucleotides, 3 to 8 nucleotides, 3 to 7 nucleotides, 3 to 6 nucleotides, 3 to 5 nucleotides, or 3 to 4 nucleotides. The extended extension primer can have an extension portion of, for example, 4 to 20 nucleotides, 4 to 19 nucleotides, 4 to 18 nucleotides, 4 to 17 nucleotides, 4 to 16 nucleotides, 4 to 15 nucleotides, 4 to 14 nucleotides, 4 to 13 nucleotides, 4 to 12 nucleotides, 4 to 11 nucleotides, 4 to 10 nucleotides, 4 to 9 nucleotides, 4 to 8 nucleotides, 4 to 7 nucleotides, 4 to 6 nucleotides, or 4 to 5 nucleotides.

The extended extension primer can have an extension portion of, for example, 5 to 20 nucleotides, 5 to 19 nucleotides, 5 to 18 nucleotides, 5 to 17 nucleotides, 5 to 16 nucleotides, 5 to 15 nucleotides, 5 to 14 nucleotides, 5 to 13 nucleotides, 5 to 12 nucleotides, 5 to 11 nucleotides, 5 to 10 nucleotides, 5 to 9 nucleotides, 5 to 8 nucleotides, 5 to 7 nucleotides, or 5 to 6 nucleotides. The extended extension primer can have an extension portion of, for example, 6 to 20 nucleotides, 6 to 19 nucleotides, 6 to 18 nucleotides, 6 to 17 nucleotides, 6 to 16 nucleotides, 6 to 15 nucleotides, 6 to 14 nucleotides, 6 to 13 nucleotides, 6 to 12 nucleotides, 6 to 11 nucleotides, 6 to 10 nucleotides, 6 to 9 nucleotides, 6 to 8 nucleotides, or 6 to 7 nucleotides. The extended extension primer can have an extension portion of, for example, 7 to 20 nucleotides, 7 to 19 nucleotides, 7 to 18 nucleotides, 7 to 17 nucleotides, 7 to 16 nucleotides, 7 to 15 nucleotides, 7 to 14 nucleotides, 7 to 13 nucleotides, 7 to 12 nucleotides, 7 to 11 nucleotides, 7 to 10 nucleotides, 7 to 9 nucleotides, or 7 to 8 nucleotides.

The extended extension primer can have an extension portion of, for example, 8 to 20 nucleotides, 8 to 19 nucleotides, 8 to 18 nucleotides, 8 to 17 nucleotides, 8 to 16 nucleotides, 8 to 15 nucleotides, 8 to 14 nucleotides, 8 to 13 nucleotides, 8 to 12 nucleotides, 8 to 11 nucleotides, 8 to 10 nucleotides, or 8 to 9 nucleotides. The extended extension primer can have an extension portion of, for example, 9 to 20 nucleotides, 9 to 19 nucleotides, 9 to 18 nucleotides, 9 to 17 nucleotides, 9 to 16 nucleotides, 9 to 15 nucleotides, 9 to 14 nucleotides, 9 to 13 nucleotides, 9 to 12 nucleotides, 9 to 11 nucleotides, or 9 to 10 nucleotides. The extended extension primer can have an extension portion of, for example, 10 to 20 nucleotides, 10 to 19 nucleotides, 10 to 18 nucleotides, 10 to 17 nucleotides, 10 to 16 nucleotides, 10 to 15 nucleotides, 10 to 14 nucleotides, 10 to 13 nucleotides, 10 to 12 nucleotides, or 10 to 11 nucleotides. The extended extension primer can have an extension portion of, for example, 11 to 20 nucleotides, 11 to 19 nucleotides, 11 to 18 nucleotides, 11 to 17 nucleotides, 11 to 16 nucleotides, 11 to 15 nucleotides, 11 to 14 nucleotides, 11 to 13 nucleotides, or 11 to 12 nucleotides.

The extended extension primer can have an extension portion of, for example, 12 to 20 nucleotides, 12 to 19 nucleotides, 12 to 18 nucleotides, 12 to 17 nucleotides, 12 to 16 nucleotides, 12 to 15 nucleotides, 12 to 14 nucleotides, or 12 to 13 nucleotides. The extended extension primer can have an extension portion of, for example, 13 to 20 nucleotides, 13 to 19 nucleotides, 13 to 18 nucleotides, 13 to 17 nucleotides, 13 to 16 nucleotides, 13 to 15 nucleotides, or 13 to 14 nucleotides. The extended extension primer can have an extension portion of, for example, 14 to 20 nucleotides, 14 to 19 nucleotides, 14 to 18 nucleotides, 14 to 17 nucleotides, 14 to 16 nucleotides, 14 to 15 nucleotides, 15 to 20 nucleotides, 15 to 19 nucleotides, 15 to 18 nucleotides, 15 to 17 nucleotides, 15 to 16 nucleotides, 16 to 20 nucleotides, 16 to 19 nucleotides, 16 to 18 nucleotides, 16 to 17 nucleotides, 17 to 20 nucleotides, 17 to 19 nucleotides, 17 to 18 nucleotides, 18 to 20 nucleotides, 18 to 19 nucleotides, or 19 to 20 nucleotides. The extended extension primer can have an extension portion of, for example, 20 nucleotides, 19 nucleotides, 18 nucleotides, 17 nucleotides, 16 nucleotides, 15 nucleotides, 14 nucleotides, 13 nucleotides, 12 nucleotides, 11 nucleotides, 10 nucleotides, 9 nucleotides, 8 nucleotides, 7 nucleotides, 6 nucleotides, 5 nucleotides, 4 nucleotides, or 3 nucleotides.

3. Sets of Extended Extension Primers

Multiple extended extension primers can be used together, such as in sets of more than one extended extension primer. Primers in such sets can have any or all of a variety of characteristics in common, that differ, or a combination. Thus, for example, a set of extended extension primers can be made up of extended extension primers corresponding to different target templates of nucleic acid sequences, extended extension primers having the same or similar hybrid stability, extended extension primers having the same or similar melting temperature, extended extension primers having a common non-target complement portion, extended extension primers having the same number of nucleotides in the extension portion, extended extension primers formed from extension primers having the same or similar hybrid stability, extended extension primers formed from extension primers having the same or similar melting temperature, or a combination of two or more of such features. Any of the disclosed characteristics of extended extension primers can be the subject of similarity or difference between extended extension primers in a set; all such sets are specifically contemplated and should be considered disclosed.

Where multiple extended extension primers are used, or for any set of extended extension primers, the melting temperature of the extended extension primers can be referred to in different ways. For example, the extended extension primers can each have a given melting temperature or range of melting temperature; one or more, two or more, three or more, etc., of the extended extension primers can have a given melting temperature or range of melting temperature; or at least one, at least two, at least three, etc., of the extended extension primers can have a given melting temperature or range of melting temperature. All of the extended extension primer melting temperatures, melting temperature ranges, and melting temperature relationships disclosed herein can be embodied in groups and sets of extended extension primers where the melting temperatures are referred to in the manner described above. Such concepts are specifically contemplated and should be considered disclosed herein. Thus, for example, the extended extension primer melting temperature range of about 0° C. to about 6° C. higher than the temperature at which the extension primers and target templates are incubated (one of the ranges described above) can be combined with the above concepts to result in a group or set of extended extension primers in which, for example, the extended extension primers can each have a melting temperature of about 0° C. to about 6° C. higher than the temperature at which the extension primers and target templates are incubated; one or more, two or more, three or more, etc., of the extended extension primers can have a melting temperature of about 0° C. to about 6° C. higher than the temperature at which the extension primers and target templates are incubated; or at least one, at least two, at least three, etc., of the extended extension primers can have a melting temperature of about 0° C. to about 6° C. higher than the temperature at which the extension primers and target templates are incubated. All such combinations are contemplated and should be considered specifically disclosed.

A useful form of extended extension primer set is a set where all or a subset of the extended extension primers have the same or similar hybrid stability or the same or similar melting temperature. Such similarity gives the extended extension primers similar thermodynamic behavior and can give the disclosed method more consistent operation when using multiple extended extension primers. The hybrid stability and/or melting temperature of extended extension primers can be calculated using known formulas and principles of thermodynamics (see, for example, Santa Lucia et al., Biochemistry 35:3555-3562 (1996); Freier et al., Proc. Natl. Acad. Sci. USA 83:9373-9377 (1986); Breslauer et al., Proc. Natl. Acad. Sci. USA 83:3746-3750 (1986)). These principles are described more fully elsewhere herein. The hybrid stability of the extended extension primers can be made more similar (a process that can be referred to as smoothing the hybrid stabilities) by, for example, chemically modifying the extended extension primers (Nguyen et al., Nucleic Acids Res. 25(15):3059-3065 (1997); Hohsisel, Nucleic Acids Res. 24(3):430-432 (1996)). Hybrid stability can also be smoothed by carrying out the hybridization under specialized conditions (Nguyen et al., Nucleic Acids Res. 27(6): 1492-1498 (1999); Wood et al., Proc. Natl. Acad. Sci. USA 82(6):1585-1588 (1985)).

Another particularly useful means of smoothing hybrid stability or melting temperature of the extended extension primers is to vary the length of the extended extension primers (or the extended target complement portion of the extended extension primers). This would allow adjustment of the hybrid stability of each extended extension primer so that all of the extended extension primers had similar hybrid stabilities (to the extent possible). Since the addition or deletion of a single nucleotide from an extended extension primer will change the hybrid stability of the extended extension primer by a fixed increment, it is understood that the hybrid stabilities of the extended extension primers typically will not be equal. For this reason, smoothing of hybrid stability of extended extension primers as used herein refers to any increase in the similarity of the hybrid stabilities of extended extension primers (or, put another way, any reduction in the differences in hybrid stabilities of the extended extension primers).

Similarity of hybrid stability of a set or group of extended extension primers can be referred to by the percent difference in stability of the extended extension primers in the set that form the most stable and least stable hybrids. Thus, a set of extended extension primers can have, for example, about 1%, about 2%, about 3%, about 4%, about 5%, about 6%, about 7%, about 8%, about 9%, about 10%, greater than about 10%, 1%, 2%, 3%, 4%, 5%, 6%, 7%, 8%, 9%, 10%, or greater than 10% difference in hybrid stability. Similarity of melting temperature of a set or group of extended extension primers can be referred to by the range of melting temperatures of the extended extension primers in the set. Thus, a set of extended extension primers can have, for example, extended extension primers with melting temperatures within about 10° C, about 9° C., about 8° C., about 7° C., about 6° C., about 5° C., about 4° C., about 3° C., about 2° C., about 1° C., 10°, 9° C., 8° C., 7° C., 6° C., 5° C., 4° C., 3° C., 2° C., or 1° C.

The efficiency of the disclosed method can also be improved by grouping extended extension primers of similar hybrid stability in different assays, reactions, and/or reaction chambers that can be subjected to different reaction conditions. In this way, the reaction conditions can be optimized for particular classes of extended extension primers.

Extended extension primers can also be designed to be produced from extension primers that have the same or similar hybrid stability or the same or similar melting temperature. Particularly useful are sets of extended extension primers where the extedned extension primers have the same or similar hybrid stability or the same or similar melting temperature and that are produced from extension primers having the same or similar hybrid stability or the same or similar melting temperature.

D. Detection Probes

Detection probes are oligonucleotides, oligonucleotide analogs, or a combination, that can interact with extended extension primers thereby facilitating detection of the extended extension primers. The extended extension primers can interact with detection probes in a nucleotide sequence-specific manner (by base pairing or hybridization, for example). Where there are different extended extension primers and/or different detection probes, the nucleotide sequence-specific interaction generally occurs only between particular extended extension primers and particular detection probes. That is, extended extension primers can be specific to particular detection probes based on the specificity of the nucleotide sequence-specific interaction. Extended extension primers having such a relationship with a given detection probe can be said to correspond to the detection probe. Similarly, the detection probes that have such a relationship with a given extended extension primer can be said to correspond to the extended extension primers.

Detection probes include a primer complement portion and, optionally, a probe complement portion and/or a stem portion. The primer complement portion of a detection probe can be complementary to all or part of the extended target complement portion of an extended extension primer, all or a part of an extended extension primer, and/or all or a part of the extension portion of an extended extension primers. The probe complement portion of a detection probe can be complementary to the probe complement portion of an anchor probe or to the stem portion of the detection probe (in a stem-loop detection probe). The primer complement portion and probe complement portion of a detection probe are generally non-overlapping and can be either abutting (that is, adjacent with no intervening nucleotides) or non-abutting. The abutting arrangement is useful for facilitating coupling an extended extension primer to the detection probe or an anchor probe.

Although detection probes can have sequences complementary to extension primers, extended extension primers generally have a higher melting temperature with their corresponding detection probe than the corresponding extension primer has with the detection probe. This relationship allows extended extension probes to interact with detection probes in preference to extension probes. As described elsewhere herein, extended extension primers generally have a higher melting temperature when interacting with their corresponding detection probe than when interacting with their corresponding target template. This relationship allows extended extension probes to interact with detection probes in preference to interaction with target templates and can help drive the amplification by abstracting extended extension primers and allowing the target template to facilitate extension of another extension primer.

Abstraction of extended extension primers can also be supported by coupling extended extension primers to detection probes. Such coupling stabilizes the interaction of the extended extension probe and the detection probe, thus preventing the extended extension primer from interacting with target templates. Coupling of the extended extension primer drives the amplification beyond the effect of the thermodynamics and kinetics of the interactions of the extended extension primer with target templates and detection probes by making the interaction with the detection probe essentially irreversible.

Coupling can be accomplished by any suitable means, including enzymatic ligation and reaction with a reactive group. Both ligation and coupling via a reactive group can couple ends of the extended extension primer and detection probe. Coupling via a reactive group can couple extended extension probes and detection probes at any point of interaction (based on the location of the reactive group). Many chemistries and techniques for coupling compounds are known and can be used to couple reporter signals to analytes. For example, coupling can be made using thiols, epoxides, nitriles for thiols, NHS esters, isothiocyanates for amines, and alcohols for carboxylic acids.

Coupling of extended extension primers to detection probes can be facilitated by stem-loop detection probes. Stem-loop detection probes are detection probes that can form a stem and loop structure where a 3′ tail extends beyond the stem. In useful stem-loop detection probes, the 3′ tail comprises or consists of the primer complement portion of the detection probe. Stem-loop detection probes where the primer complement portion extends to the stem facilitate coupling of extended extension primers to the detection probes by placing the 3′ end of the extended extension primers adjacent to the 5′ end of the detection probe (that is, the free end of the stem). This allows ligation. For coupling via a chemically reactive group, the 5′ end of the stem-loop detection probe can be derivatized with a reactive group.

Detection probes can be associated with a solid supports as described elsewhere herein. In some embodiments, detection probes can contain a spacer. The spacer, if used, can help to overcome steric factors from the surface if the detection probe is immobilized, aid in anchoring ligase on detection probes, or provide other advantages, such as control or alteration of the hydrophobicity of detection probes attached to a solid support. Spacers useful for the disclosed method include nucleotide spacers such as poly dT or poly dA; aliphatic linkers such as C18, C12, or multimers thereof, aromatic spacers, or RNA, DNA, PNA or combinations thereof.

E. Anchor Probes

Anchor probes are oligonucleotides, oligonucleotide analogs, or a combination, that can interact with detection probes and that can be coupled to extended extension primers. The anchor probes can interact with detection probes in a nucleotide sequence-specific manner (by base pairing or hybridization, for example). Where there are different anchor probes and/or different detection probes, the nucleotide sequence-specific interaction generally occurs only between particular anchor probes and particular detection probes. That is, anchor probes can be specific to particular detection probes based on the specificity of the nucleotide sequence-specific interaction. Anchor probes having such a relationship with a given detection probe can be said to correspond to the detection probe. Similarly, the detection probes that have such a relationship with a given anchor probe can be said to correspond to the anchor probes. Anchor probes that correspond to a given detection probe can be said to correspond to extended extension probes that correspond to that detection probe. Similarly, extended extension primers that correspond to a given detection probe can be said to correspond to anchor probes that correspond to that detection probe.

Anchor probes include a probe complement portion. The probe complement portion of an anchor probe can be complementary to the probe complement portion of a detection probe. Abstraction of extended extension primers from interaction with target templates can be supported by coupling extended extension primers to anchor probes. Such coupling stabilizes the interaction of the extended extension probe and the detection probe to which the anchor probe interacts, thus preventing the extended extension primer from interacting with target templates. Coupling of the extended extension primer drives the amplification beyond the effect of the thermodynamics and kinetics of the interactions of the extended extension primer with target templates and detection probes by making the interaction with the detection probe and/or anchor probe essentially irreversible.

Coupling can be accomplished by any suitable means, including enzymatic ligation and reaction with a reactive group. Both ligation and coupling via a reactive group can couple ends of the extended extension primer and detection probe. Coupling via a reactive group can couple extended extension probes and detection probes at any point of interaction (based on the location of the reactive group). Many chemistries and techniques for coupling compounds are known and can be used to couple reporter signals to analytes. For example, coupling can be made using thiols, epoxides, nitrites for thiols, NHS esters, isothiocyanates for amines, and alcohols for carboxylic acids.

Coupling of extended extension primers to anchor probes can be facilitated by interaction of anchor probes and extended extension primers to detection probes. The interaction of detection probes with both extended extension probes and anchor probes brings the extended extension probe into proximity with the anchor probe, thus allowing the extended extension primer and anchor probe to be coupled. The extended extension primer interaction with the primer complement portion of the detection probe and the anchor probe interacts with the probe complement portion of the detection probe. In some detection probes, the primer complement portion and probe complement portion are abutting (that is, adjacent with no intervening nucleotides). The abutting arrangement brings the extended extension primer and anchor probe into an arrangement that allows coupling of their ends.

Anchor probes can be associated with a solid supports as described elsewhere herein. In some embodiments, anchor probes can contain a spacer. The spacer, if used, can help to overcome steric factors from the surface if the anchor probe is immobilized, aid in anchoring ligase on anchor probes, or provide other advantages, such as control or alteration of the hydrophobicity of anchor probes attached to a solid support. Spacers useful for the disclosed method include nucleotide spacers such as poly dT or poly dA; aliphatic linkers such as C18, C12, or multimers thereof; aromatic spacers, or RNA, DNA, PNA or combinations thereof.

F. Nucleic Acid Sequences

The disclosed method involves use of nucleic acid molecules and nucleic acid sequences for amplification as target templates or for production of target templates. As used herein, unless the context indicates otherwise, the term nucleic acid molecule refers to both actual molecules and to nucleic acid sequences that are part of a larger nucleic acid molecule. Nucleic acid molecules and nucleic acid sequences used as, and/or to produce, target templates are also referred to as nucleic acid sequences of interest and target sequences. Examples of nucleic acid molecules or sources of nucleic acid molecules and target templates include mRNA molecules, cDNA molecules, genomic DNA, viral nucleic acid, and cloned nucleic acid, although any nucleic acid molecule or sequence can be used in the disclosed compositions and method. Nucleic acid molecules, which can be used as target templates and/or the source for production of target templates, can be any nucleic acid. Nucleic acid molecules can include multiple nucleic acid molecules, such as in the case of mRNA amplification, multiple sites in a nucleic acid molecule, or a single region of a nucleic acid molecule. For example, nucleic acid molecules can be mRNA and cDNA.

Target sequences can be from any nucleic acid sample of interest. The source, identity, and preparation of many such nucleic acid samples are known. It is useful if nucleic acid samples known or identified for use in amplification or detection methods are used for the method described herein. The nucleic acid sample can be, for example, a nucleic acid sample from one or more cells, tissue, or bodily fluids such as blood, urine, semen, lymphatic fluid, cerebrospinal fluid, or amniotic fluid, or other biological samples, such as tissue culture cells, buccal swabs, mouthwash, stool, tissues slices, biopsy aspiration, and archeological samples such as bone or mummified tissue. Types of useful nucleic acid samples include blood samples, urine samples, semen samples, lymphatic fluid samples, cerebrospinal fluid samples, amniotic fluid samples, biopsy samples, needle aspiration biopsy samples, cancer samples, tumor samples, tissue samples, cell samples, cell lysate samples, a crude cell lysate samples, forensic samples, archeological samples, infection samples, nosocomial infection samples, production samples, drug preparation samples, biological molecule production samples, protein preparation samples, lipid preparation samples, and/or carbohydrate preparation samples.

G. Nucleic Acid Samples

Nucleic acid samples can be derived from any source that has, or is suspected of having, nucleic acids. A nucleic acid sample can be a source of nucleic acids used to produce target templates. The nucleic acid sample can contain target nucleic acids, for example specific mRNA molecules or pool of mRNA molecules. Nucleic acid samples can contain RNA or DNA or both. Nucleic acid samples in certain embodiments can also include chemically synthesized nucleic acids. Nucleic acid samples can include any nucleotide, nucleotide analog; nucleotide substitute or nucleotide conjugate.

Nucleic acid samples can be, for example, a nucleic acid sample from one or more cells, tissue, or bodily fluids such as blood, urine, semen, lymphatic fluid, cerebrospinal fluid, or amniotic fluid, or other biological samples, such as tissue culture cells, buccal swabs, mouthwash, stool, tissues slices, biopsy aspiration, and archeological samples such as bone or mummified tissue. Types of useful nucleic acid samples include blood samples, urine samples, semen samples, lymphatic fluid samples, cerebrospinal fluid samples, amniotic fluid samples, biopsy samples, needle aspiration biopsy samples, cancer samples, tumor samples, tissue samples, cell samples, cell lysate samples, crude cell lysate samples, forensic samples, archeological samples, infection samples, nosocomial infection samples, production samples, drug preparation samples, biological molecule production samples, protein preparation samples, lipid preparation samples, and/or carbohydrate preparation samples.

H. Nucleic Acid Libraries

The disclosed method can be used to produce extended extension primers and/or derivatives of extended extension primers that serve as a nucleic acid library of a nucleic acid sample. Such a nucleic acid library can be used for any purpose, including, for example, detection of sequences, production of probes, production of nucleic acid arrays or chips, and comparison with nucleic acids in other nucleic acid libraries. Similarly prepared nucleic acid libraries of other nucleic acid samples can allow convenient detection of differences between the samples. Nucleic acid libraries can be used both for detection of related nucleic acid samples and comparison of nucleic acid samples. For example, the presence or identity of specific organisms can be detected by producing a nucleic acid library of the test organism and comparing the resulting nucleic acid library with reference nucleic acid libraries prepared from known organisms. Changes and differences in gene expression patterns can also be detected by preparing nucleic acid libraries of mRNA from different cell samples and comparing the nucleic acid libraries. The extended extension primers can also be used to produce a set of probes or primers that is specific for the source of a nucleic acid sample. The extended extension primers can also be used as a fingerprint of nucleic acid sequences present in a sample. Nucleic acid libraries can be made up of, or derived from, for example, the mRNA of a sample such that the entire relevant mRNA content of the sample is substantially represented.

Nucleic acid libraries can be stored or archived for later use. For example, extended extension primers produced in the disclosed method can be physically stored, either in solution, frozen, or attached or adhered to a solid-state substrate such as an array. Storage in an array is useful for providing an archived probe set derived from the nucleic acids in any sample of interest. As another example, informational content of, or derived from, nucleic acid fingerprints can also be stored. Such information can be stored, for example, in or as computer readable media. Examples of informational content of nucleic acid libraries include nucleic acid sequence information (complete or partial); differential nucleic acid sequence information such as sequences present in one sample but not another; hybridization patterns of extended extension primers to, for example, nucleic acid arrays, sets, chips, or other extended extension primers. Numerous other data that is or can be derived from nucleic acid libraries and extended extension primers produced in the disclosed method can also be collected, used, saved, stored, and/or archived.

Nucleic acid libraries can also contain or be made up of other information derived from the information generated in the disclosed method, and can be combined with information obtained or generated from any other source. The informational nature of nucleic acid libraries produced using the disclosed method lends itself to combination and/or analysis using known bioinformatics systems and methods.

Nucleic acid libraries of nucleic acid samples can be compared to similar nucleic acid libraries derived from any other sample to detect similarities and differences in the samples (which is indicative of similarities and differences in the nucleic acids in the samples). For example, a nucleic acid library of a first nucleic acid sample can be compared to a nucleic acid library of a sample from the same type of organism as the first nucleic acid sample, a sample from the same type of tissue as the first nucleic acid sample, a sample from the same organism as the first nucleic acid sample, a sample obtained from the same source but at time different from that of the first nucleic acid sample, a sample from an organism different from that of the first nucleic acid sample, a sample from a type of tissue different from that of the first nucleic acid sample, a sample from a strain of organism different from that of the first nucleic acid sample, a sample from a species of organism different from that of the first nucleic acid sample, or a sample from a type of organism different from that of the first nucleic acid sample.

The same type of tissue is tissue of the same type such as liver tissue, muscle tissue, or skin (which may be from the same or a different organism or type of organism). The same organism refers to the same individual, animal, or cell. For example, two samples taken from a patient are from the same organism. The same source is similar but broader, referring to samples from, for example, the same organism, the same tissue from the same organism, the same DNA molecule, or the same DNA library. Samples from the same source that are to be compared can be collected at different times (thus allowing for potential changes over time to be detected). This is especially useful when the effect of a treatment or change in condition is to be assessed. Samples from the same source that have undergone different treatments can also be collected and compared using the disclosed method. A different organism refers to a different individual organism, such as a different patient, a different individual animal. Different organism includes a different organism of the same type or organisms of different types. A different type of organism refers to organisms of different types such as a dog and cat, a human and a mouse, or E. coli and Salmonella. A different type of tissue refers to tissues of different types such as liver and kidney, or skin and brain. A different strain or species of organism refers to organisms differing in their species or strain designation as those terms are understood in the art.

I. Fragmentation Primers

Fragmentation primers are primers that that are used to produce replicated strands that can be cleaved at defined sequences. Fragmentation primers include a target complement portion and, optionally, a non-complementary portion. Fragmentation primers can be used to produce defined 5′ ends that serve as replication terminating features. Generally, fragmentation primers can be designed to hybridize to and prime replication from a defined sequence. The site or sites of cleavage generally can be specified by the location of specific nucleotides, modified nucleotides, or other component incorporated into the fragmentation primer. For example, deoxyuridine nucleotides can be included in a fragmentation primer. The replicated strand produced from the fragmentation primer can then be cleaved at the site of the deoxyuridine nucleotides (as described elsewhere herein).

J. Detection Labels

To aid in detection and quantitation of extended extension primers produced in the disclosed method, detection labels can be included in or coupled to extension primers or directly incorporated into extended extension primers. As used herein, a detection label is any molecule that can be associated with nucleic acid, directly or indirectly, and which results in a measurable, detectable signal, either directly or indirectly. Many such labels for incorporation into nucleic acids or coupling to nucleic acids are known to those of skill in the art. Examples of detection labels suitable for use in the disclosed method are radioactive isotopes, fluorescent molecules, phosphorescent molecules, enzymes, antibodies, and ligands.

Examples of suitable fluorescent labels include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, Cascade Blue®, Oregon Green®, pyrene, lissamine, xanthenes, acridines, oxazines, phycoerythrin, macrocyclic chelates of lanthanide ions such as quantum dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy F1, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH—CH₃, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin E8G, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

Useful fluorescent labels are fluorescein (5-carboxyfluorescein-N-hydroxysuccinimide ester), rhodamine (5,6-tetramethyl rhodamine), and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including Amersham Pharmacia Biotech, Piscataway, N.J.; Molecular Probes, Eugene, Oreg.; and Research Organics, Cleveland, Ohio.

Additional labels of interest include those that provide for signal only when the nucleic acid with which they are associated is specifically bound to a target molecule. Such labels include “molecular beacons” as described in Tyagi & Kramer, Nature Biotechnology (1996) 14:303 and EP 0 070 685 B1. Other labels of interest include those described in U.S. Pat. No. 5,563,037; WO 97/17471 and WO 97/17076.

Labeled nucleotides are a useful form of detection label since they can be directly incorporated into the extension primers or other nucleic acids during synthesis. Examples of detection labels that can be incorporated into nucleic acids include nucleotide analogs such as BrdUrd (5-bromodeoxyuridine, Hoy and Schimke, Mutation Research 290:217-230 (1993)), aminoallyldeoxyuridine (Henegariu et al., Nature Biotechnology 18:345-348 (2000)), 5-methylcytosine (Sano et al., Biochim. Biophys. Acta 951:157-165 (1988)), bromouridine (Wansick et al., J. Cell Biology 122:283-293 (1993)) and nucleotides modified with biotin (Langer et al., Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is BrdUrd (bromodeoxyuridine, BrdUrd, BrdU, BUdR, Sigma-Aldrich Co). Other useful nucleotide analogs for incorporation of detection label into DNA are AA-dUTP (aminoallyl-deoxyuridine triphosphate, Sigma-Aldrich Co.), and 5-methyl-dCTP (Roche Molecular Biochemicals). A preferred nucleotide analog for incorporation of detection label into RNA is biotin- 16-UTP (biotin-16-uridine-5′-triphosphate, Roche Molecular Biochemicals). Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Detection labels that are incorporated into nucleic acid, such as biotin, can be subsequently detected using sensitive methods known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2,-dioxetane-3-2′-(5′-chloro)tricyclo [3.3.1.1^{3,7}]decane]-4-yl) phenyl phosphate; Tropix, Inc.). Labels can also be enzymes, such as alkaline phosphatase, soybean peroxidase, horseradish peroxidase and polymerases, that can be detected, for example, with chemical signal amplification or by using a substrate to the enzyme which produces light (for example, a chemiluminescent 1,2-dioxetane substrate) or fluorescent signal.

Molecules that combine two or more of these detection labels are also considered detection labels. Any of the known detection labels can be used with the disclosed primers, probes, tags, and method to label and detect extended extension primers produced in the disclosed method. Methods for detecting and measuring signals generated by detection labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection label coupled to the antibody.

K. Oligonucleotide Synthesis

Extension primers, detection probes, anchor probes, target templates, and any other oligonucleotides can be synthesized using established oligonucleotide synthesis methods. Methods to produce or synthesize oligonucleotides are well known in the art. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method. Target templates also can be produced from nucleic acid molecules by techniques described elsewhere herein. Solid phase chemical synthesis of DNA fragments can be performed using protected nucleoside cyanoethyl phosphoramidites (S. L. Beaucage et al. (1981) Tetrahedron Lett. 22:1859). In this approach, the 3′-hydroxyl group of an initial 5′-protected nucleoside is first covalently attached to the polymer support (R. C. Pless et al. (1975) Nucleic Acids Res. 2:773 (1975)). Synthesis of the oligonucleotide then proceeds by deprotection of the 5′-hydroxyl group of the attached nucleoside, followed by coupling of an incoming nucleoside-3′-phosphoramidite to the deprotected hydroxyl group (M. D. Matteucci et a. (1981) J. Am. Chem. Soc. 103:3185). The resulting phosphite triester is finally oxidized to a phosphorotriester to complete the internucleotide bond (R. L. Letsinger et al. (1976) J. Am. Chem. Soc. 9:3655). Alternatively, the synthesis of phosphorothioate linkages can be carried out by sulfurization of the phosphite triester. Several chemicals can be used to perform this reaction, among them 3H-1,2-benzodithiole-3-one, 1,1-dioxide (R. P. Iyer, W. Egan, J. B. Regan, and S. L. Beaucage, J. Am. Chem. Soc., 1990, 112, 1253-1254). The steps of deprotection, coupling and oxidation are repeated until an oligonucleotide of the desired length and sequence is obtained. Other methods exist to generate oligonucleotides such as the H-phosphonate method (Hall et al, (1957) J. Chem. Soc., 3291-3296) or the phosphotriester method as described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994). Other forms of oligonucleotide synthesis are described in U.S. Pat. No. 6,294,664 and U.S. Pat. No. 6,291,669.

Many of the oligonucleotides described herein are designed to be complementary to certain portions of other oligonucleotides or nucleic acids such that stable hybrids can be formed between them via base pairing. The stability of these hybrids can be calculated using known methods such as those described in Santa Lucia et al., Biochemistry 35:3555-3562 (1996); Freier et al., Proc. Natl. Acad. Sci. USA 83:9373-9377 (1986); Breslauer et al., Proc. Natl. Acad. Sci. USA 83:3746-3750 (1986), Lesnick and Freier, Biochemistry 34:10807-10815 (1995), McGraw et al., Biotechniques 8:674-678 (1990), and Rychlik et al., Nucleic Acids Res. 18:6409-6412 (1990).

Oligonucleotides can be synthesized, for example, on a Perseptive Biosystems 8909 Expedite Nucleic Acid Synthesis system using standard β-cyanoethyl phosphoramidite coupling chemistry on synthesis columns (Glen Research, Sterling, Va.). Oxidation of the newly formed phosphites can be carried out using, for example, the sulfurizing reagent 3H-1,2-benzothiole-3-one-1,1-idoxide (Glen Research) or the standard oxidizing reagent after the first and second phosphoramidite addition steps. The thio-phosphitylated oligonucleotides can be deprotected, for example, using 30% ammonium hydroxide (3.0 ml) in water at 55° C. for 16 hours, concentrated in an OP 120 Savant Oligo Prep deprotection unit for 2 hours, and desalted with PD10 Sephadex columns using the protocol provided by the manufacturer.

So long as their relevant function is maintained, extension primers, detection probes, anchor probes, target templates, and any other oligonucleotides can be made up of or include modified nucleotides (nucleotide analogs). Many modified nucleotides are known and can be used in oligonucleotides. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl, hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Other modified bases are those that function as universal bases. Universal bases include 3-nitropyrrole and 5-nitroindole. Universal bases substitute for the normal bases but have no bias in base pairing. That is, universal bases can base pair with any other base. Base modifications often can be combined with for example a sugar modification, such as 2′-O-methoxyethyl, to achieve useful properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference in its entirety, and specifically for their description of base modifications, their synthesis, their use, and their incorporation into oligonucleotides and nucleic acids.

Nucleotide analogs can also include modifications of the sugar moiety. Modifications to the sugar moiety would include natural modifications of the ribose and deoxyribose as well as synthetic modifications. Sugar modifications include but are not limited to the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10, alkyl or C2 to C10 alkenyl and alkynyl. 2′ sugar modifications also include but are not limited to —O[(CH₂)n O]m CH₃, —O(CH₂)n OCH₃, —O(CH₂)n NH₂, —O(CH₂)n CH₃, —O(CH₂)n —ONH2, and —O(CH₂)nON[(CH ₂)n CH₃)]₂, where n and m are from 1 to about 10.

Other modifications at the 2′ position include but are not limited to: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmnacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety, and specifically for their description of modified sugar structutres, their synthesis, their use, and their incorporation into nucleotides, oligonucleotides and nucleic acids.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkages between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference its entirety, and specifically for their description of modified phosphates, their synthesis, their use, and their incorporation into nucleotides, oligonucleotides and nucleic acids.

It is understood that nucleotide analogs need only contain a single modification, but may also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize and hybridize to (base pair to) complementary nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference its entirety, and specifically for their description of phosphate replacements, their synthesis, their use, and their incorporation into nucleotides, oligonucleotides and nucleic acids.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science 254:1497-1500 (1991)).

Oligonucleotides can be comprised of nucleotides and can be made up of different types of nucleotides or the same type of nucleotides. For example, one or more of the nucleotides in an oligonucleotide can be ribonucleotides, 2′-O-methyl ribonucleotides, or a mixture of ribonucleotides and 2′-O-methyl ribonucleotides; about 10% to about 50% of the nucleotides can be ribonucleotides, 2′-O-methyl ribonucleotides, or a mixture of ribonucleotides and 2′-O-methyl ribonucleotides; about 50% or more of the nucleotides can be ribonucleotides, 2′-O-methyl ribonucleotides, or a mixture of ribonucleotides and 2′-O-methyl ribonucleotides; or all of the nucleotides are ribonucleotides, 2′-O-methyl ribonucleotides, or a mixture of ribonucleotides and 2′-O-methyl ribonucleotides. Such oligonucleotides can be referred to as chimeric oligonucleotides.

A random or degenerate sequence refers to the relationship between different sequences such that, collectively, the sequences encompass a set of related sequences. Thus, a fully random sequence (or fully degenerate sequence) refers to a set of sequences where every possible sequence is represented. A partially random sequence (or partially degenerate sequence) refers to a set of sequences where (1) some base positions are the same in all of the sequences and some base positions differ between the sequences, and/or (2) more than one but less than every possible sequence is represented. The term random sequence can refer to either or both fully random and partially random sequences. A given molecule can contain both random and non-random portions (that is, random and non-random sequences). It should be understood that reference to a random sequence or nucleic acid molecule refers both to a single sequence or nucleic acid molecule in a set of random sequences or nucleic acid molecules or to a set of sequences or nucleic acid molecules that collectively have random sequences as described above. In general, the context will indicate whether a single sequence or nucleic acid molecule or multiple sequence or nucleic acid molecules are being referred to. Sets of sequences or nucleic acid molecules having random or partially random sequences can be synthesized using standard techniques by allowing the addition of any nucleotide at each position to be randomized. It should be understood that due to limitations of the synthetic process used, synthesis of fully random sequences may not, in fact, produce a set containing every possible sequence. As a practical matter, such sequences, or, more properly, such sequence sets), are intended to be encompassed by the term fully random sequence so long as synthesis of a fully random sequence was the goal. In any case, such sets of sequences are also encompassed by the term partially random.

L. Solid Supports

Solid supports are solid-state substrates or supports with which detection probes, anchor probes, extended extension primers or other components used in or with the disclosed method can be associated. Detection probe, anchor probes, and extended extension primers can be associated with solid supports directly or indirectly. For example, extended extension primers can be bound to the surface of a solid support or associated with anchor probes and/or detection probes immobilized on solid supports. An array detector is a solid support to which multiple different anchor probes and/or detection probes have been coupled in an array, grid, or other organized or specified pattern.

Solid-state substrates for use in solid supports can include any solid material with which components can be associated, directly or indirectly. This includes materials such as acrylamide, agarose, cellulose, cellulose, nitrocellulose, glass, gold, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A useful form for a solid-state substrate is a microtiter dish. A particularly useful form of microtiter dish is the standard 96-well type. In some embodiments, a multiwell glass slide can be employed.

In some embodiments of the disclosed method, amplification is performed in solution and the resulting extended extension primers are captured on a solid support. For example, the multiple extension primers can be extended together (that is, not in separate reaction chambers) and the products (extended extension primers) captured. For example, extension can be performed to produce extended extension primers, and the extended extension primers then can be detected or quantitated by hybridization to a solid support containing detection probes complementary to the extended extension primers. By attaching different detection probes to different regions of a solid support, different extended extension primers can be captured at different, and therefore diagnostic, locations on the solid support. For example, in a microtiter plate multiplex assay, detection probes specific for up to 96 different extended extension primers (each produced from a different target template or target template sequence) can be immobilized on a microtiter plate, each in a different well. Capture and detection will occur only in those probe elements on the solid support corresponding to extended extension primers for which the corresponding target templates (or nucleic acids from which the target templates were derived) were present in a sample.

Different anchor probes and/or detection probes can be used together as a set. The set can be used as a mixture of all or subsets of the anchor probes and/or detection probes used separately in separate reactions, or immobilized on a solid support. Anchor probes and/or detection probes used separately or as mixtures can be physically separable through, for example, association with or immobilization on a solid support. An array can include a plurality of anchor probes and/or detection probes immobilized at identified or predefined locations on the solid support. Each predefined location on the solid support generally has one type of component (that is, all the components at that location are the same). Alternatively, multiple types of components can be immobilized in the same predefined location on a solid support. Each location will have multiple copies of the given components. The spatial separation of different components on the solid support allows separate detection and identification of amplification products.

Although useful, it is not required that the solid support be a single unit or structure. The set of anchor probes, detection probes and/or other components may be distributed over any number of solid supports. For example, at one extreme, each probe may be immobilized in a separate reaction tube or container, or on separate beads or microparticles.

Methods for immobilization of oligonucleotides to solid-state substrates are well established. Oligonucleotides, including anchor probes and detection probes, can be coupled to substrates using established coupling methods. For example, suitable attachment methods are described by Pease et al., Proc. Natl. Acad. Sci. USA 91(11):5022-5026 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A useful method of attaching oligonucleotides to solid-state substrates is described by Guo et al., Nucleic Acids Res. 22:5456-5465 (1994).

Each of the components (for example, anchor probes and/or detection probes) immobilized on the solid support can be located in a different predefined region of the solid support. The different locations can be different reaction chambers. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances.

Components can be associated or immobilized on a solid support at any density. Components can be immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

M. DNA Polymerases

The disclosed method makes use of DNA polymerase to extend extension primers and otherwise synthesize DNA. Many DNA polymerases are known and any suitable DNA polymerase can be used. Different forms of the method, and particular steps in the method, are best performed with DNA polymerase having particular activities and/or characteristics. For example, it is useful to use a DNA polymerase that will stop replicating at or because of a replication terminating feature for extension of extension primers. Although many replication terminating features work with any polymerase (5′ ends, for example), other replication terminating features may function only with some polymerases. For example, many, but not all, DNA polymerases require a DNA template, so ribonucleotides in an otherwise deoxyribonucleotide target template could serve as replication terminating features. Useful DNA polymerases include bacteriophage Deep Vent DNA polymerase (Huang and Keohavong, “Fidelity and predominant mutations produced by deep vent wild-type and exonuclease-deficient DNA polymerases during in vitro DNA amplification” DNA Cell Biol. 15:589-94 (1996)) and ThermoSequenase DNA polymerase (Amersham Biosciences). Other useful polymerases include exo(−)VENT® DNA polymerase (Kong et al., J. Biol. Chem. 268:1965-1975 (1993)). Deep Vent DNA polymerase and ThermoSequenase DNA polymerase are most useful.

The suitablility of any polymerase for use in the disclosed method can be easily determined by performing the disclosed method using the polymerase to be tested. For example, one or more of the reactions as described in the Example can be performed using the polymerase to be tested rather than DeepVent(exo−) DNA polymerase (NEB) or Vent(exo−) DNA polymerase.

N. Ligases

Any DNA ligase is suitable for use in the disclosed method. Useful ligases are those that preferentially form phosphodiester bonds at nicks in double-stranded DNA. That is, ligases that fail to ligate the free ends of single-stranded DNA at a significant rate are useful. Many suitable ligases are known, such as T4 DNA ligase (Davis et al., Advanced Bacterial Genetics—A Manual for Genetic Engineering (Cold Spring Harbor Laboratory, Cold Spring Harbor, N.Y., 1980)), E. coli DNA ligase (Panasnko et al., J. Biol. Chem. 253:4590-4592 (1978)), AMPLIGASE® (Kalin et al., Mutat. Res., 283(2):119-123 (1992); Winn-Deen et al., Mol Cell Probes (England) 7(3):179-186 (1993)), Taq DNA ligase (Barany, Proc. Natl. Acad. Sci. USA 88:189-193 (1991), Thermus thermophilus DNA ligase (Abbott Laboratories), Thermus scotoductus DNA ligase and Rhodothermus marinus DNA ligase (Thorbjarnardottir et al., Gene 151:177-180 (1995)). T4 DNA ligase is useful for ligations involving probes hybridized to RNA sequences due to its ability to ligate DNA ends involved in DNA:RNA hybrids (Hsuih et al., Quantitative detection of HCV RNA using novel ligation-dependent polymerase chain reaction, American Association for the Study of Liver Diseases (Chicago, Ill., Nov. 3-7, 1995)).

O. Reverse Transcriptases

Reverse transcriptases useful for producing nucleic acid molecules and target templates for use in the disclosed method, or for extending extension primers, can be any polymerase that exhibits reverse transcriptase activity. Useful catalytic activities include an RNA-dependent DNA polymerase activity and a RNase H activity. The reverse transcriptase can also have a DNA-dependent DNA polymerase activity for second strand synthesis, although a separate DNA polymerase can be used. Second strand synthesis is not required to produce target templates or to extend extension primers. Most reverse transcriptases, including those derived from Moloney murine leukemia virus (MMLV-RT), avian myeloblastosis virus (AMV-RT), bovine leukemia virus (BLV-RT), Rous sarcoma virus (RSV) and human immunodeficiency virus (HIV-RT) have each of these activities. Many other reverse transcriptases are known (available from, for example, Boehringer Mannheim Corp., Indianapolis, Ind.; Life Technologies, Inc., Rockville, Md.; New England Biolabs, Inc., Beverley, Mass.; Perkin Elmer Corp., Norwalk, Conn.; Pharmacia LKB Biotechnology, Inc., Piscataway, N.J.; Qiagen, Inc., Valencia, Calif.; Stratagene, La Jolla, Calif.) which can be used with the disclosed method. A variety of proteins that catalyze one or two of these activities can be added to the cDNA synthesis reaction. For example, MMLV reverse transcriptase lacking RNase H activity (described in U.S. Pat. No. 5,405,776) which catalyzes RNA-dependent DNA polymerase activity and DNA-dependent DNA polymerase activity, can be added with a source of RNase H activity, such as the RNase H purified from cellular sources, including Escherichia coli. These proteins may be added together during a single reaction step, or added sequentially during two or more substeps. RNase H activity is not required to produce target templates or to extend extension primers. Finally, additional proteins that may enhance the yield of double-stranded DNA products (if double-stranded products are desired) may also be added to the cDNA synthesis reaction. These proteins include a variety of DNA polymerases (such as those derived from E coli, thermophilic bacteria, archaebacteria, phage, yeasts, Neurosporas, Drosophilas, primates and rodents), and DNA Ligases (such as those derived from phage or cellular sources, including T4 DNA Ligase and E. coli DNA Ligase).

P. Kits

The materials described above can be packaged together in any suitable combination as a kit useful for performing the disclosed method. It is useful if the kit components in a given kit is designed and adapted for use together in the disclosed method. For example disclosed are kits for producing extended extension primers, the kit comprising one or more extension primers. The kits also can contain one or more enzymes to be used in the method, reagents for producing target templates, and/or buffers and other reagents.

The disclosed kits can also include one or more detection probes, one or more anchor probes and/or one or more target templates. Preferably, a portion of each of the detection probes in a kit has sequence matching or complementary to a portion of a different extended extension primer to be detected.

Q. Mixtures

Disclosed are mixtures formed by performing the disclosed method. For example, disclosed are mixtures comprising target templates and extension primers; extension primers and extended extension primers; target templates and extended extension primers; target templates, extension primers and extended extension primers; extended extension primers and detection probes; extension primers, extended extension primers and detection probes; target templates, extended extension primers and detection probes; target templates, extension primers, extended extension primers and detection probes; extended extension primers and anchor probes; extension primers, extended extension primers and anchor probes; target templates, extended extension primers and anchor probes; target templates, extension primers, extended extension primers and anchor probes; extended extension primers, anchor probes and detection probes; extension primers, extended extension primers, anchor probes and detection probes; or target templates, extended extension primers, anchor probes and detection probes; target templates, extension primers, extended extension primers, anchor probes and detection probes.

Whenever the method involves mixing or bringing into contact compositions or components or reagents, performing the method creates a number of different mixtures. For example, if the method includes 3 mixing steps, after each one of these steps a unique mixture is formed if the steps are performed sequentially. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

R. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising reactions or components of the method in combination with an incubator or temperature regulating device; or reactions or components of the method in combination with a device for detecting and/or quantitating detection labels.

S. Data Structures and Computer Control

Disclosed are data structures used in, generated by, or generated from, the disclosed method. Data structures generally are any form of data, information, and/or objects collected, organized, stored, and/or embodied in a composition or medium. A nucleic acid library stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Uses

The disclosed methods and compositions are applicable to numerous areas including, but not limited to, analysis of nucleic acids present in a sample (for example, analysis of messenger RNA in a sample), disease detection, mutation detection, gene expression profiling, RNA expression profiling, gene discovery, gene mapping (molecular haplotyping), agricultural research, and virus detection. The disclosed method can be used to analyze of messenger RNA expression. Other uses include detection of nucleic acids in situ in cells, on microarrays, on DNA fibers, and on genomic DNA arrays; detection of RNA in cells; RNA expression profiling; molecular haplotyping; mutation detection; detection of abnormal RNA (for example, overexpression of an oncogene or absence of expression of a tumor suppressor gene); expression in cancer cells; detection of viral genome in cells; viral RNA expression; detection of inherited diseases such as cystic fibrosis, muscular dystrophy, diabetes, hemophilia, sickle cell anemia; assessment of predisposition for cancers such as prostate cancer, breast cancer, lung cancer, colon cancer, ovarian cancer, testicular cancer, pancreatic cancer.

### Method

Disclosed is a method for amplifying nucleic acid sequences by limited primer extension. The disclosed method involves association of a primer with a template, extension of the primer for a short distance, termination of extension, and dissociation of the primer from the template, whereupon the events repeat with a new primer. The repeated association, extension, and dissociation of primers from a single template sequence results in amplification of the extended sequences. The termination of extension can be effected by a feature of the template sequence. The reaction can be carried under a single set of conditions, such as isothermal conditions, based on the thermodynamics of dissociation of the extended primers. At temperatures around the melting temperature of the extended primer, including temperatures below the melting temperature, a significant fraction of the extended primer will be dissociated at any one time (put another way, each template sequence will be free of primer a fraction of the time). This allows a new, unextended primer to become associated with the template sequence and extended. The kinetics of the amplification can be improved by reducing the chance of reassociation of extended primers with the target sequence. This can be accomplished, for example, by providing a thermodynamically favored sink for the extended primers, by using an excess of unextended primer over the number of extended primers that will be produced, or both. A thermodynamically favored sink can be, for example, a peptide nucleic acid complementary to the extended primer or ligation of the extended primer to another nucleic acid.

The disclosed method is particularly suited to detection of nucleic acid sequences. By amplifying specific sequences targeted by extension primers and adjacent to replication terminating features, extended extension primers are produced incorporating the targeted sequences. By detecting the extended extension primers, the corresponding targeted sequence is detected. If a targeted sequence is not present (or if there is no adjacent replication terminating feature), the sequence will not be amplified, no extended extension primer corresponding to that sequence will be produced or detected, and thus the sequence will not be detected.

Multiple sequences can be amplified in the same reaction by targeting multiple sequences with extension primers. Likewise, multiple sequences can be detected via production of their corresponding extended extension primers. Multiplex detection is facilitated by the different sequences of different extension primers. For example, the extended extension primers can be hybridized to detection probes. Detection probes can also serve as sink for extended extension primers during the amplification reaction. In this way, sequences can be detected during the amplification reaction. Such simultaneous amplification and detection can be facilitated by using detection probes associated with a substrate. Multiplex detection can be facilitated by an array of detection probes with different detection probes at different locations of a substrate.

The sequences amplified depend on the extension primers; only those sequences targeted by an extension primer (and adjacent to a replication terminating feature) will be amplified. This allows specific sequences to be targeted for amplification. Flexibility in the location of replication terminating features allows flexibility in targeting sequences. If a targeted sequence is not present (or if there is no adjacent replication terminating feature), the sequence will not be amplified.

The extension primers and target templates can be incubated at any suitable temperature. Generally, extension primers and target templates should be incubated at a temperature that (in combination with other conditions of the reaction) promote interaction of the extension primers and the target templates, extension of the extension primers using the interacting target templates as template, and dissociation of the extended extension primers from the target templates, whereby multiple extended extension primers are produced from at least one target template. As described elsewhere herein, these effect can be obtained and increased by the use of extension primer and target templates that have certain relationships to each other and to the resulting extended extension primers. These effects can also be aided by performing extension at a temperatures with particular relationships to the melting temperature of, for example, extension primers, target templates, extended extension primers and detection probes. Thus, the temperature at which extension primers and target templates are incubated can be expressed in terms of the difference from the melting temperature of extension primers and target templates. Although the following description refers to the temperature at which extension primers and target templates are incubated in relation to the melting temperature of extension primers, this description should also be understood to disclose and describe the temperature at which extension primers and target templates are incubated in relation to the melting temperature of the target complement portion of extension primers, the temperature at which extension primers and target templates are incubated in relation to the melting temperature of extension primers when interacting with target templates, and the temperature at which extension primers and target templates are incubated in relation to the melting temperature of extension primers when interacting with target templates to which the extension primers correspond.

The temperature at which the extension primers and target templates are incubated can be, for example, about 5° C. to about 20° C., about 5° C. to about 19° C., about 5° C. to about 18° C., about 5° C. to about 17° C., about 5° C. to about 16° C., about 5° C. to about 15° C., about 5° C. to about 14° C., about 5° C. to about 13° C., about 5° C. to about 12° C., about 5° C. to about 11° C., about 5° C. to about 10° C., about 5° C. to about 9° C., about 5° C. to about 8° C., about 5° C. to about 7° C., or about 5° C. to about 6° C. higher than the melting temperature of the extension primer.

The temperature at which the extension primers and target templates are incubated can be, for example, about 6° C. to about 20° C., about 6° C. to about 19° C., about 6° C. to about 18° C., about 6° C. to about 17° C., about 6° C. to about 16° C., about 6° C. to about 15° C., about 6° C. to about 14° C., about 6° C. to about 13° C., about 6° C. to about 12° C., about 6° C. to about 11° C., about 6° C. to about 10° C., about 6° C. to about 9° C., about 6° C. to about 8° C., or about 6° C. to about 7° C. higher than the melting temperature of the extension primer. The temperature at which the extension primers and target templates are incubated can be, for example, about 7° C. to about 20° C., about 7° C. to about 19° C., about 7° C. to about 18° C., about 7° C. to about 17° C., about 7° C. to about 16° C., about 7° C. to about 15° C., about 7° C. to about 14° C., about 7° C. to about 13° C., about 7° C. to about 12° C., about 7° C. to about 11° C., about 7° C. to about 10° C., about 7° C. to about 9° C., or about 7° C. to about 8° C. higher than the melting temperature of the extension primer.

The temperature at which the extension primers and target templates are incubated can be, for example, about 8° C. to about 20° C., about 8° C. to about 19° C., about 8° C. to about 18° C., about 8° C. to about 17° C., about 8° C. to about 16° C., about 8° C. to about 15° C., about 8° C. to about 14° C., about 8° C. to about 13° C., about 8° C. to about 12° C., about 8° C. to about 11° C., about 8° C. to about 10° C., or about 8° C. to about 9° C. higher than the melting temperature of the extension primer. The temperature at which the extension primers and target templates are incubated can be, for example, about 9° C. to about 20° C., about 9° C. to about 19° C., about 9° C. to about 18° C., about 9° C. to about 17° C., about 9° C. to about 16° C., about 9° C. to about 15° C., about 9° C. to about 14° C., about 9° C. to about 13° C., about 9° C. to about 12° C., about 9° C. to about 11° C., or about 9° C. to about 10° C. higher than the melting temperature of the extension primer.

The temperature at which the extension primers and target templates are incubated can be, for example, about 10° C. to about 20° C., about 10° C. to about 19° C., about 10° C. to about 18° C., about 10° C. to about 17° C., about 10° C. to about 16° C., about 10° C. to about 15° C., about 10° C. to about 14° C., about 10° C. to about 13° C., about 10° C. to about 12° C., or about 10° C. to about 11° C. higher than the melting temperature of the extension primer. The temperature at which the extension primers and target templates are incubated can be, for example, about 11° C. to about 20° C., about 11° C. to about 19° C., about 11° C. to about 18° C., about 11° C. to about 17° C., about 11° C. to about 16° C., about 11° C. to about 15° C., about 11° C. to about 14° C., about 11° C. to about 13° C., or about 11° C. to about 12° C. higher than the melting temperature of the extension primer. The temperature at which the extension primers and target templates are incubated can be, for example, about 12° C. to about 20° C., about 12° C. to about 19° C., about 12° C. to about 18° C., about 12° C. to about 17° C., about 12° C. to about 16° C., about 12° C. to about 15° C., about 12° C. to about 14° C., or about 12° C. to about 13° C. higher than the melting temperature of the extension primer.

The temperature at which the extension primers and target templates are incubated can be, for example, about 13° C. to about 20° C., about 13° C. to about 19° C., about 13° C. to about 18° C., about 13° C. to about 17° C., about 13° C. to about 16° C., about 13° C. to about 15° C., or about 13° C. to about 14° C. higher than the melting temperature of the extension primer. The temperature at which the extension primers and target templates are incubated can be, for example, about 14° C. to about 20° C., about 14° C. to about 19° C., about 14° C. to about 18° C., about 14° C. to about 17° C., about 14° C. to about 16° C., or about 14° C. to about 15° C. higher than the melting temperature of the extension primer. The temperature at which the extension primers and target templates are incubated can be, for example, about 15° C. to about 20° C., about 15° C. to about 19° C., about 15° C. to about 18° C., about 15° C. to about 17° C., or about 15° C. to about 16° C. higher than the melting temperature of the extension primer.

The temperature at which the extension primers and target templates are incubated can be, for example, about 16° C. to about 20° C., about 16° C. to about 19° C., about 16° C. to about 18° C., about 16° C. to about 17° C., 17° C. to about 20° C., about 17° C. to about 19° C., about 17° C. to about 18° C., about 18° C. to about 20° C., about 18° C. to about 19° C., or about 19° C. to about 20° C. higher than the melting temperature of the extension primer. The temperature at which the extension primers and target templates are incubated can be, for example, about 20° C., about 19° C., about 18° C., about 17° C., about 16° C., about 15° C., about 14° C., about 13° C., about 12° C., about 11° C., about 10° C., about 9° C., about 8° C., about 7° C., about 6° C., or about 5° C. higher than the melting temperature of the extension primer. The temperature at which the extension primers and target templates are incubated can be, for example, 20° C., 19° C., 18° C., 17° C., 16° C., 15° C., 14° C., 13° C., 12° C., 11° C., 10° C., 9° C., 8° C., 7° C., 6° C., or 5° C. higher than the melting temperature of the extension primer. All of the above relationships also can be described in terms of the melting temperature of the extension primer being lower than the temperature at which the extension primer and target templates are incubated, and such alternative descriptions are contemplated and should be considered specifically disclosed.

The temperature at which the extension primer and target templates are incubated can also be expressed in terms of the difference from the melting temperature of extended extension primers. Although the following description refers to the temperature at which the extension primer and target templates are incubated in relation to the melting temperature of extended extension primers, this description should also be understood to disclose and describe the temperature at which the extension primer and target templates are incubated in relation to the melting temperature of the extended target complement portion of extended extension primers, the temperature at which the extension primer and target templates are incubated in relation to the melting temperature of entire extended extension primers, the temperature at which the extension primer and target templates are incubated in relation to the melting temperature of extended extension primers when interacting with target templates, and the temperature at which the extension primer and target templates are incubated in relation to the melting temperature of extended extension primers when interacting with target templates to which the extended extension primers correspond.

The temperature at which the extension primer and target templates are incubated can be, for example, about 0° C. to about 20° C., about 0° C. to about 19° C., about 0° C. to about 18° C., about 0° C. to about 17° C., about 0° C. to about 16° C., about 0° C. to about 15° C., about 0° C. to about 14° C., about 0° C. to about 13° C., about 0° C. to about 12° C., about 0° C. to about 11° C., about 0° C. to about 10° C., about 0° C. to about 9° C., about 0° C. to about 8° C., about 0° C. to about 7° C., about 0° C. to about 6° C., about 0° C. to about 5° C., about 0° C. to about 4° C., about 0° C. to about 3° C., about 0° C. to about 2° C., or about 0° C. to about 1° C. lower than the melting temperature of the extended extension primer.

The temperature at which the extension primer and target templates are incubated can be, for example, about 1° C. to about 20° C., about 1° C. to about 19° C., about 1° C. to about 18° C., about 1° C. to about 17° C., about 1° C. to about 16° C., about 1° C. to about 15° C., 1° C. to about 14° C., about 1° C. to about 13° C., about 1° C. to about 12° C., about 1° C. to about 11° C., about 1° C. to about 10° C., about 1° C. to about 9° C., about 1° C. to about 8° C., about 1° C. to about 7° C., about 1° C. to about 6° C., about 1° C. to about 5° C., about 1° C. to about 4° C., about 1° C. to about 3° C., or about 1° C. to about 2° C. lower than the melting temperature of the extended extension primer. The temperature at which the extension primer and target templates are incubated can be, for example, about 2° C. to about 20° C., about 2° C. to about 19° C., about 2° C. to about 18° C., about 2° C. to about 17° C., about 2° C. to about 16° C., 2° C. to about 15° C., about 2° C. to about 14° C., about 2° C. to about 13° C., about 2° C. to about 12° C., about 2° C. to about 11° C., about 2° C. to about 10° C., about 2° C. to about 9° C., about 2° C. to about 8° C., about 2° C. to about 7° C., about 2° C. to about 6° C., about 2° C. to a about 2° C. to about 4° C., or about 2° C. to about 3° C. lower than the melting temperature of the extended extension primer.

The temperature at which the extension primer and target templates are incubated can be, for example, about 3° C. to about 20° C., about 3° C. to about 19° C., about 3° C. to about 18° C., about 3° C. to about 17° C., about 3° C. to about 16° C., about 3° C. to about 15° C., 3° C. to about 14° C., about 3° C. to about 13° C., about 3° C. to about 12° C., about 3° C. to about 11° C., about 3° C. to about 10° C., about 3° C. to about 9° C., about 3° C. to about 8° C., about 3° C. to about 7° C., about 3° C. to about 6° C., about 3° C. to about 5° C., or about 3° C. to about 4° C. lower than the melting temperature of the extended extension primer. The temperature at which the extension primer and target templates are incubated can be, for example, about 4° C. to about 20° C., about 4° C. to about 19° C., about 4° C. to about 18° C., about 4° C. to about 17° C., about 4° C. to about 16° C., about 4° C. to about 15° C., about 4° C. to about 14° C., 4° C. to about 13° C., about 4° C. to about 12° C., about 4° C. to about 11° C., about 4° C. to about 10° C., about 4° C. to about 9° C., about 4° C. to about 8° C., about 4° C. to about 7° C., about to about 6° C., or about 4° C. to about 5° C. lower than the melting temperature of the extended extension primer.

The temperature at which the extension primer and target templates are incubated can be, for example, about 5° C. to about 20° C., about 5° C. to about 19° C., about 5° C. to about 18° C., about 5° C. to about 17° C., about 5° C. to about 16° C., about 5° C. to about 15° C., 5° C. to about 14° C., about 5° C. to about 13° C., about 5° C. to about 12° C., about 5° C. to about 11° C., about 5° C. to about 10° C., about 5° C. to about 9° C., about 5° C. to about 8° C., about to about 7° C., or about 5° C. to about 6° C. lower than the melting temperature of the extended extension primer.

The temperature at which the extension primer and target templates are incubated can be, for example, about 6° C. to about 20° C., about 6° C. to about 19° C., about 6° C. to about 18° C., about 6° C. to about 17° C., about 6° C. to about 16° C., about 6° C. to about 15° C., 6° C. to about 14° C., about 6° C. to about 13° C., about 6° C. to about 12° C., about 6° C. to about 11° C., about 6° C. to about 10° C., about 6° C. to about 9° C., about 6° C. to about 8° C., 6° C. to about 7° C. lower than the melting temperature of the extended extension primer. The temperature at which the extension primer and target templates are incubated can be, for example, about 7° C. to about 20° C., about 7° C. to about 19° C., about 7° C. to about 18° C., about 7° C. to about 17° C., about 7° C. to about 16° C., about 7° C. to about 15° C., about 7° C. to about 14° C., about 7° C. to about 13° C., about 7° C. to about 12° C., about 7° C. to about 11° C., about 7° C. to about 10° C., about 7° C. to about 9° C., or about 7° C. to about 8° C. lower than the melting temperature of the extended extension primer.

The temperature at which the extension primer and target templates are incubated can be, for example, about 8° C. to about 20° C., about 8° C. to about 19° C., about 8° C. to about 18° C., about 8° C. to about 17° C., about 8° C. to about 16° C., about 8° C. to about 15° C., 8° C. to about 14° C., about 8° C. to about 13° C., about 8° C. to about 12° C., about 8° C. to about 11° C., about 8° C. to about 10° C., or about 8° C. to about 9° C. lower than the melting temperature of the extended extension primer. The temperature at which the extension primer and target templates are incubated can be, for example, about 9° C. to about 20° C., about 9° C. to about 19° C., about 9° C. to about 18° C., about 9° C. to about 17° C., about 9° C., about 16° C., about 9° C. to about 15° C., about 9° C. to about 14° C., about 9° C. to about 13° C., about 9° C. to about 12° C., about 9° C. to about 1 I° C., or about 9° C. to about 10° C. lower than the melting temperature of the extended extension primer.

The temperature at which the extension primer and target templates are incubated can be, for example, about 10° C. to about 20° C., about 10° C. to about 19° C., about 10° C. to about 18° C., about 10° C. to about 17° C., about 10° C. to about 16° C., about 10° C. to about 15° C., about 10° C. to about 14° C., about 10° C. to about 13° C., about 10° C. to about 12° C., about 10° C. to about 11° C. lower than the melting temperature of the extended extension primer. The temperature at which the extension primer and target templates are incubated can be, for example, about 1°° C. to about 20° C., about 11° C. to about 19° C., about 11° C. to about 18° C., about 11° C. to about 17° C., about 11° C. to about 16° C., about 11° C. to about 15° C., about 1°° C. to about 14° C., about 1° C. to about 13° C., or about 11° C. to about 12° C. lower than the melting temperature of the extended extension primer. The temperature at which the extension primer and target templates are incubated can be, for example, about 12° C. to about 20° C., about 12° C. to about 19° C., about 12° C. to about 18° C., about 12° C. to about 17° C., about 12° C. to about 16° C., about 12° C. to about 15° C., about 12° C. to about 14° C., or about 12° C. to about 13° C. lower than the melting temperature of the extended extension primer.

The temperature at which the extension primer and target templates are incubated can be, for example, about 13° C. to about 20° C., about 13° C. to about 19° C., about 13° C. to about 18° C., about 13° C. to about 17° C., about 13° C. to about 16° C., about 13° C. to about 15° C., or about 13° C. to about 14° C. lower than the melting temperature of the extended extension primer. The temperature at which the extension primer and target templates are incubated can be, for example, about 14° C. to about 20° C., about 14° C. to about 19° C., about 14° C. to about 18° C., about 14° C. to about 17° C., about 14° C. to about 16° C., or about 14° C. to about 15° C. lower than the melting temperature of the extended extension primer. The temperature at which the extension primer and target templates are incubated can be, for example, about 15° C. to about 20° C., about 15° C. to about 19° C., about 15° C. to about 18° C., about 15° C. to about 17° C., or about 15° C. to about 16° C. lower than the melting temperature of the extended extension primer.

The temperature at which the extension primer and target templates are incubated can be, for example, about 16° C. to about 20° C., about 16° C. to about 19° C., about 16° C. to about 18° C., about 16° C. to about 17° C., 17° C. to about 20° C., about 17° C. to about 19° C., about 17° C. to about 18° C., about 18° C. to about 20° C., about 18° C. to about 19° C., or about 19° C. to about 20° C. lower than the melting temperature of the extended extension primer. The temperature at which the extension primer and target templates are incubated can be, for example, about 20° C., about 19° C., about 18° C., about 17° C., about 16° C., about 15° C., about 14° C., about 13° C., about 12° C., about 11° C., about 10° C., about 9° C., about 8° C., about 7° C., about 6° C., about 5° C., about 4° C., about 3° C., about 2° C., about 1° C., about 0° C. lower than the melting temperature of the extended extension primer. The temperature at which the extension primer and target templates are incubated can be, for example, 20° C., 19° C., 18° C., 17° C., 16° C., 15° C., 14° C., 13° C., 12° C., 11° C., 10° C., 9° C., 8° C., 7° C., 6° C., 5° C., 4° C., 3° C., 2° C., 1° C., or 0° C. lower than the melting temperature of the extended extension primer. All of the above relationships also can be described in terms of the melting temperature of extended extension primers being higher than the temperature at which the extension primer and target templates are incubated, and such alternative descriptions are contemplated and should be considered specifically disclosed.

A. Signal Quotients

The disclosed method produces signals based on extended extension primers. That is, in the method, production of extended extension primers is detected, either directly or indirectly. Modes and means of such detection are described elsewhere herein. The signals produced can be processed in order to gain information about extended extension primers and the target templates from which they are derived. For example, signals for extended extension primers generated form genomic target templates can be used to assess the presence, absence and amplification of alleles in the genome. One useful way to process signals generated in the disclosed method is through Signal Quotients (SQ).

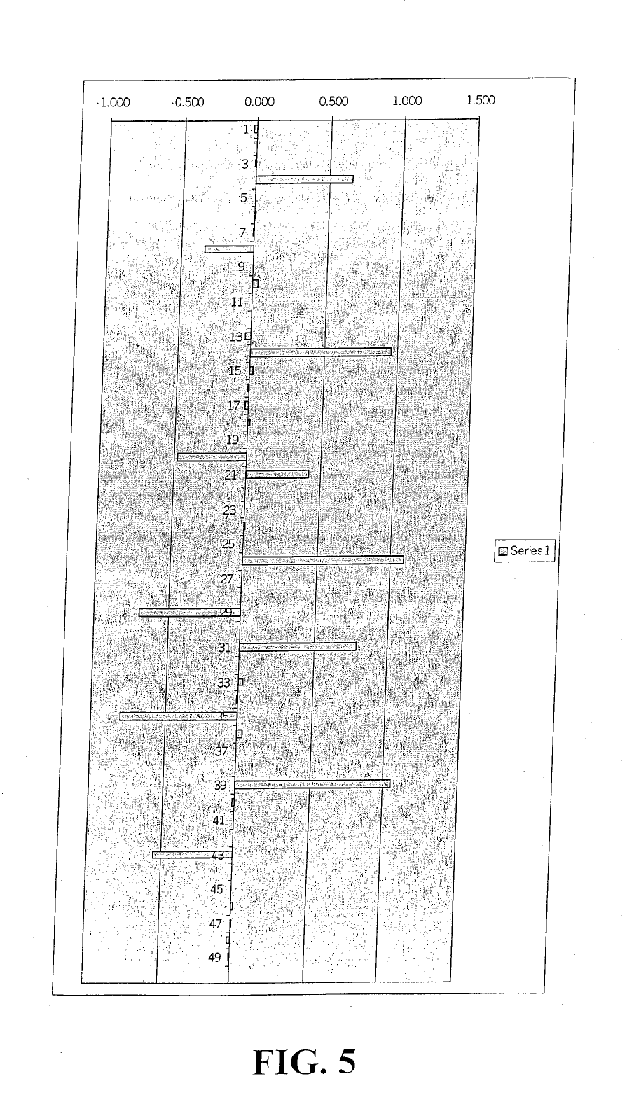

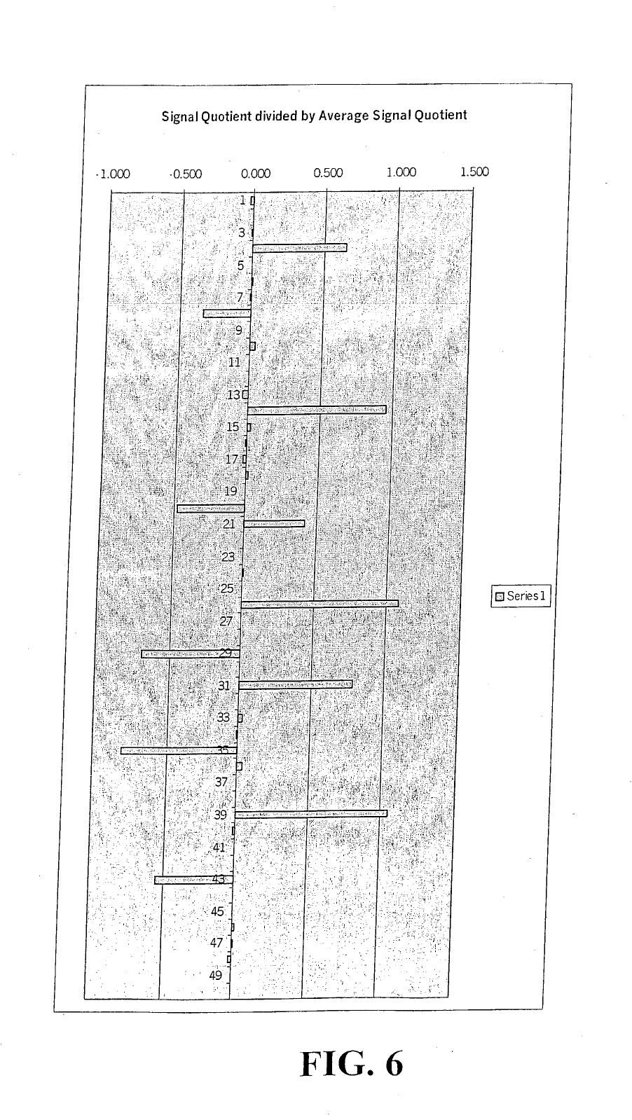

Signals can be processed by calculating a Ratio of Signal Quotients (RSQ) where RSQ=Median Signal Quotient (MSQ) divided by Average Signal Quotient (ASQ). The Ratio of Signal Quotients (RSQ) can be, for example, plotted to show gains and losses of alleles across the genome. Examples of such plots are shown in FIGS. 5 and 6. The RSQ can be analyzed or assessed in any other suitable manner.

The Median Signal Quotient can be calculated by dividing the signal intensity from a specific extended extension primer by the median of the signal intensities from all extended extension primers being assessed, measured or otherwise detected. These calculations can be used when multiple target templates are assessed in the same, for example, operation, assay, or experiment.

The Average Signal Quotient can be calculated using signal intensities obtained from calibration assays. A calibration assay is a form of the disclosed method performed using normal DNA and the same reagents and procedure that are used for other assays (with which the results from the calibration assay will be used) using nucleic acid samples of interest. Specifically, the Average Signal Quotient can be calculated by dividing the average signal intensity from a specific extended extension primer over a number of calibration assays by the average signal intensity from all extended extension primers for all of the calibration assays. The calibration assays generally will be separate assays performed using the same label and same reagents using DNA from normal individuals. The Average Signal Quotient represents the relative signal intensity of specific extended extension primers, compared to the total ensemble of extended extension primers, in a calibration assay. The average of many measurements is calculated in order to account for possible variations in results.

Generally, the more calibration assays performed, the better the results. For example, 10 or more or 16 or more calibration assays would be a useful number. Experimental error decreases with the square root of the number of observations. Thus, for 9 observations the observed error is expected to be three times lower than for a single observation. For 16 observations, the observed error is expected to be four times lower than for a single observation. The more observations used for this calculation, the lower the expected error. Thus, performing more experiments for use in calculating the ASQ can provide better results.

Table 1 a model example of signal data and calculated signal quotients based on the signal data. The model data represent signals from detection of 50 different extended extension primers corresponding to 50 different target templates. In this example, the data are modeled to represent signals that could be obtained from target templates derived from a genomic DNA sample. The Signal column is the raw signal obtained when the extended extension primers are detected. The median signal and the average signal for the fifty signals are listed at the bottom of the Signal column. The MSQ column is the Median Signal Quotient for each extended extension primer and is calculated as described above (that is, signal divided by median signal). The RSQ column is the Ratio of Signal Quotients and represents the Median Signal Quotient divided by the Average Signal Quotient. The Average Signal Quotient would be based on the results of a series of calibration assays as described above. The log(RSQ) column is the log of RSQ for each extended extension primer. The log(RSQ) for each extended extension primer is plotted in FIG. 5.

TABLE 1

| Primer | Signal | MSQ | RSQ | log (RSQ) |
|---|---|---|---|---|
| 1 | 288 | 0.664 | 0.948 | −0.023 |
| 2 | 202 | 0.465 | 0.999 | 0.000 |
| 3 | 234 | 0.539 | 0.988 | −0.005 |
| 4 | 1544 | 3.558 | 4.489 | 0.652 |
| 5 | 435 | 1.002 | 0.999 | 0.000 |
| 6 | 354 | 0.816 | 1.025 | 0.011 |
| 7 | 234 | 0.539 | 0.987 | −0.006 |
| 8 | 210 | 0.484 | 0.460 | −0.337 |
| 9 | 433 | 0.998 | 0.997 | −0.001 |
| 10 | 367 | 0.846 | 1.095 | 0.040 |
| 11 | 354 | 0.816 | 0.999 | 0.000 |
| 12 | 234 | 0.539 | 0.999 | 0.000 |
| 13 | 599 | 1.380 | 0.915 | −0.038 |
| 14 | 2028 | 4.673 | 8.962 | 0.952 |
| 15 | 460 | 1.060 | 1.055 | 0.023 |
| 16 | 324 | 0.747 | 0.989 | −0.005 |
| 17 | 501 | 1.154 | 0.945 | −0.025 |
| 18 | 358 | 0.825 | 1.032 | 0.014 |
| 19 | 454 | 1.046 | 0.995 | −0.002 |
| 20 | 198 | 0.456 | 0.335 | −0.474 |
| 21 | 1694 | 3.903 | 2.617 | 0.418 |
| 22 | 543 | 1.251 | 0.999 | 0.000 |
| 23 | 234 | 0.539 | 0.999 | 0.000 |
| 24 | 543 | 1.251 | 1.014 | 0.006 |
| 25 | 555 | 1.279 | 0.996 | −0.002 |
| 26 | 4190 | 9.654 | 12.173 | 1.085 |
| 27 | 234 | 0.539 | 0.992 | −0.004 |
| 28 | 545 | 1.256 | 0.996 | −0.002 |
| 29 | 99 | 0.228 | 0.205 | −0.688 |
| 30 | 234 | 0.539 | 0.999 | 0.000 |
| 31 | 3030 | 6.982 | 6.091 | 0.785 |
| 32 | 444 | 1.023 | 1.008 | 0.004 |
| 33 | 254 | 0.585 | 1.076 | 0.032 |
| 34 | 252 | 0.581 | 0.989 | −0.005 |
| 35 | 89 | 0.205 | 0.157 | −0.804 |
| 36 | 299 | 0.689 | 1.083 | 0.035 |
| 37 | 446 | 1.028 | 0.999 | 0.000 |
| 38 | 554 | 1.276 | 1.007 | 0.003 |
| 39 | 2678 | 6.171 | 11.253 | 1.051 |
| 40 | 733 | 1.689 | 0.969 | −0.014 |
| 41 | 665 | 1.532 | 0.999 | 0.000 |
| 42 | 254 | 0.585 | 1.006 | 0.003 |
| 43 | 156 | 0.359 | 0.280 | −0.553 |
| 44 | 634 | 1.461 | 1.005 | 0.002 |
| 45 | 557 | 1.283 | 1.001 | 0.000 |
| 46 | 399 | 0.919 | 1.036 | 0.016 |
| 47 | 675 | 1.555 | 1.016 | 0.007 |
| 48 | 821 | 1.892 | 0.946 | −0.024 |
| 49 | 354 | 0.816 | 0.991 | −0.004 |
| 50 | 654 | 1.507 | 1.001 | 0.001 |
| Median | 434 | | | |
| Average | 653 | | | |

The data include some signals that reflect increased or decreased signal relative to the “normal” signal for that at extended extension primer (signals for extended extension primers 4, 14, 21, 31 and 39 are abnormally higher and signals for extended extension primers 8, 20, 29 35 and 43 are abnormally lower). Such differences result in log(RSQ) values that are of clearly greater magnitude than log(RSQ) values from “normal” signals. This can be seen in FIG. 5 where the extended extension primers with altered signals stand out.

Table 2 a model example of signal data and calculated signal quotients based on the signal data. The signal data is the same as the signal data in Table 1 except that the signals are only 0.66 of the corresponding signal in Table 1. This was done to illustrate the effect variability in assay yield would have on signal quotient calculations. The columns and their calculation are the same as in Table 1. The log(RSQ) for each extended extension primer based on the data in Table 2 is plotted in FIG. 6. As can be seen, the change in yield did not affect a Median Signal Quotient, Ration of Signal Quotients or log(RSQ). Thus, use of signal quotients to process and analyze signal data can reduce or eliminate the effects of variability in yield between assays.

TABLE 2

| | |
|---|---|
| Primer | Signal |

| | | | |
|---|---|---|---|
| (0.66 depressed) | MSQ | RSQ | log (RSQ) |

| | | | | |
|---|---|---|---|---|
| 1 | 190 | 0.664 | 0.948 | −0.023 |
| 2 | 133 | 0.465 | 0.999 | 0.000 |
| 3 | 154 | 0.539 | 0.988 | −0.005 |
| 4 | 1019 | 3.558 | 4.489 | 0.652 |
| 5 | 287 | 1.002 | 0.999 | 0.000 |
| 6 | 234 | 0.816 | 1.025 | 0.011 |
| 7 | 154 | 0.539 | 0.987 | −0.006 |
| 8 | 139 | 0.484 | 0.460 | −0.337 |
| 9 | 286 | 0.998 | 0.997 | −0.001 |
| 10 | 242 | 0.846 | 1.095 | 0.040 |
| 11 | 234 | 0.816 | 0.999 | 0.000 |
| 12 | 154 | 0.539 | 0.999 | 0.000 |
| 13 | 395 | 1.380 | 0.915 | −0.038 |
| 14 | 1338 | 4.673 | 8.962 | 0.952 |
| 15 | 304 | 1.060 | 1.055 | 0.023 |
| 16 | 214 | 0.747 | 0.989 | −0.005 |
| 17 | 331 | 1.154 | 0.945 | −0.025 |
| 18 | 236 | 0.825 | 1.032 | 0.014 |
| 19 | 300 | 1.046 | 0.995 | −0.002 |
| 20 | 131 | 0.456 | 0.335 | −0.474 |
| 21 | 1118 | 3.903 | 2.617 | 0.418 |
| 22 | 358 | 1.251 | 0.999 | 0.000 |
| 23 | 154 | 0.539 | 0.999 | 0.000 |
| 24 | 358 | 1.251 | 1.014 | 0.006 |
| 25 | 366 | 1.279 | 0.996 | −0.002 |
| 26 | 2765 | 9.654 | 12.173 | 1.085 |
| 27 | 154 | 0.539 | 0.992 | −0.004 |
| 28 | 360 | 1.256 | 0.996 | −0.002 |
| 29 | 65 | 0.228 | 0.205 | −0.688 |
| 30 | 154 | 0.539 | 0.999 | 0.000 |
| 31 | 2000 | 6.982 | 6.091 | 0.785 |
| 32 | 293 | 1.023 | 1.008 | 0.004 |
| 33 | 168 | 0.585 | 1.076 | 0.032 |
| 34 | 166 | 0.581 | 0.989 | −0.005 |
| 35 | 59 | 0.205 | 0.157 | −0.804 |
| 36 | 197 | 0.689 | 1.083 | 0.035 |
| 37 | 294 | 1.028 | 0.999 | 0.000 |
| 38 | 366 | 1.276 | 1.007 | 0.003 |
| 39 | 1767 | 6.171 | 11.253 | 1.051 |
| 40 | 484 | 1.689 | 0.969 | −0.014 |
| 41 | 439 | 1.532 | 0.999 | 0.000 |
| 42 | 168 | 0.585 | 1.006 | 0.003 |
| 43 | 103 | 0.359 | 0.280 | −0.553 |
| 44 | 418 | 1.461 | 1.005 | 0.002 |
| 45 | 368 | 1.283 | 1.001 | 0.000 |
| 46 | 263 | 0.919 | 1.036 | 0.016 |
| 47 | 446 | 1.555 | 1.016 | 0.007 |
| 48 | 542 | 1.892 | 0.946 | −0.024 |
| 49 | 234 | 0.816 | 0.991 | −0.004 |
| 50 | 432 | 1.507 | 1.001 | 0.001 |
| Median | 286.44 | | | |
| Average | 431 | | | |

### Specific Embodiments

In some forms, the method can comprise bringing into contact one or more extension primers and one or more target templates and incubating under conditions that promote interaction of the extension primers and the target templates, extension of the extension primers using the interacting target templates as template, and dissociation of the extended extension primers from the target templates, whereby multiple extended extension primers are produced from at least one target template. The target templates each comprise a replication-terminating feature.

In some forms, the method can comprise bringing into contact one or more extension primers and one or more target templates and incubating under conditions that promote interaction of the extension primers and the target templates, extension of the extension primers and one of the interacting target templates as template, and dissociation of the extended extension primers from the target templates, and detecting one or more of the extended extension primers. Multiple extended extension primers can be produced from at least one target temolate. Each extension primer can correspond to one or more of the target templates. The target templates can correspond to nucleic acid sequences of interest. Detection of an extended extension primer can indicate detection of the nucleic acid sequence of interest to which a target template corresponding to the detected extended extension primer corresponds.

In some forms, the method can comprise bringing into contact one or more extension primers and one or more target templates and incubating under conditions that promote interaction of the extension primers and the target templates, extension of the extension primers using the interacting target templates as template, and dissociation of the extended extension primers from the target templates, and detecting one or more of the extended extension primers. Multiple extended extension primers can be produced from at least one target template. Each extension primer can correspond to one or more of the target templates. The target templates can correspond to nucleic acid sequences of interest. Detection of an extended extension primer indicates detection of the nucleic acid sequence of interest to which a target template corresponding to the detected extended extension primer corresponds.

In some forms, the method can comprise bringing into contact one or more extension primers and one or more target templates and incubating under conditions that promote interaction of the extension primers and the target templates, extension of the extension primers using the interacting target templates as template, and dissociation of the extended extension primers from the target templates. Multiple extended extension primers can be produced from at least one target template.

The extension primers and target templates can be incubated under isothermal conditions. The extension primers and target templates can be incubated under a single set of conditions. The target templates are nucleic acid sequences of interest. The extension primers each can include a target complement portion. The extension primers each can comprise nucleotides consisting of a target complement portion. The extension primers each can also comprise a non-target complement portion.

The target complement portion of at least one of the extension primers can have a melting temperature of about 5° C. to about 16° C. lower than the temperature at which the extension primers and target templates are incubated. The target complement portion of at least one of the extension primers can have a melting temperature of about 10° C. to about 16° C. lower than the temperature at which the extension primers and target templates are incubated.

The target complement portion of at least one of the extension primers can have a melting temperature of about 11° C. to about 13° C. lower than the temperature at which the extension primers and target templates are incubated. The target complement portion of each extension primer can have a melting temperature of about 10° C. to about 16° C. lower than the temperature at which the extension primers and target templates are incubated. The target complement portion of each extension primer can have a calculated melting temperature of about 10° C. to about 16° C. lower than the temperature at which the extension primers and target templates are incubated. The target complement portion of at least one of the extension primers can have a melting temperature of about 10° C. to about 16° C. lower than the temperature at which the extension primers and target templates are incubated, and the extended extension primers produced by extension of the at least one extension primer can have a melting temperature of about 0° C. to about 6° C. higher than the temperature at which the extension primers and target templates are incubated. The target complement portion of each of the extension primers can have a melting temperature of about 10° C. to about 16° C. lower than the temperature at which the extension primers and target templates are incubated, and each of the extended extension primers can have a melting temperature of about 0° C. to about 6° C. higher than the temperature at which the extension primers and target templates are incubated. The melting temperature of the target complement portion of each of the extension primers can be about 10° C. to about 22° C. lower than the melting temperature of the extended extension primer.

The extension primers and target templates can be incubated at about 68° C. to about 74° C. The target complement portion of each extension primer can have a melting temperature of about 55° C. to about 61° C. The extension primers and target templates can be incubated at about 70° C. to about 72° C. The target complement portion of each extension primer can have a melting temperature of about 55° C. to about 61° C. The extension primers and target templates can be incubated at about 68° C. to about 74° C. The target complement portion of each extension primer can have a melting temperature of about 55° C. to about 61° C. The melting temperature of each extended extension primer can be about 72° C. to about 77° C. The extension primers and target templates can be incubated at about 70° C. to about 72° C. The target complement portion of each extension primer can have a melting temperature of about 55° C. to about 61° C. The melting temperature of each extended extension primer can be about 74° C. to about 76° C. At least one of the extended extension primers can have a melting temperature of about 2° C. lower than to about 7° C. higher than the temperature at which the extension primers and target templates are incubated. At least one of the extended extension primers can have a melting temperature of about 0° C. to about 6° C. higher than the temperature at which the extension primers and target templates are incubated. At least one of the extended extension primers can have a melting temperature of about 2° C. to about 5° C. higher than the temperature at which the extension primers and target templates are incubated. The extended extension primers can have melting temperatures of about 0° C. to about 6° C. higher than the temperature at which the extension primers and target templates are incubated. The extended extension primers can have calculated melting temperatures of about 0° C. to about 6° C. higher than the temperature at which the extension primers and target templates are incubated. The extension primers and target templates are incubated at about 68° C. to about 74° C., wherein the melting temperature of each extended extension primer is about 72° C. to about 77° C. The extension primers and target templates can be incubated at about 70° C. to about 72° C. The melting temperature of each extended extension primer can be about 74° C. to about 76° C.

The extension primers can be extended 5 to 11 nucleotides. The extension primers can be extended 5 to 8 nucleotides.

The extended extension primers can have melting temperatures of about 0° C. to about 6° C. higher than the temperature at which the extension primers and target templates are incubated.

The extended extension primers can each comprise an extension portion that can comprise nucleotides added to the extension primer by extension. The extended extension primers can each comprise an extended target complement portion, and the extended target complement portion can comprise the target complement portion and the extension portion. The extended extension primers can each further comprise a non-target complement portion. The extension primers can each further comprise a non-target complement portion, and the non-target complement portion of an extended extension primer can be the non-target complement portion of the extension primer from which the extended extension primer is produced. The extended extension primers each consist of an extended target complement portion. The extended extension primers can each comprise nucleotides, and the nucleotides can consist of the extended target complement portion.

The target complement portion of each of the extension primers can have a melting temperature of about 10° C. to about 16° C. lower than the temperature at which the extension primers and target templates are incubated. The extended target complement portion of each of the extended extension primers can have a melting temperature of about 0° C. to about 6° C. higher than the temperature at which the extension primers and target templates are incubated. The melting temperature of the target complement portion of each of the extension primers can be about 10° C. to about 22° C. lower than the melting temperature of the extended target complement portion of the extended extension primer.

The extension primers and target templates can be incubated at about 68° C. to about 74° C. The target complement portion of each extension primer can have a melting temperature of about 55° C. to about 61° C. The melting temperature of the extended target complement portion of each extended extension primer can be about 72° C. to about 77° C. The extended target complement portions of the extended extension primers can have melting temperatures of about 0° C. to about 6° C. higher than the temperature at which the extension primers and target templates are incubated.

At least 50 extended extension primers can be produced from at least one of the target templates. At least 100 extended extension primers can be produced from at least one of the target templates. At least 200 extended extension primers can be produced from at least one of the target templates. At least 400 extended extension primers can be produced from at least one of the target templates. At least 800 extended extension primers are produced from at least one of the target templates. At least 1000 extended extension primers are produced from at least one of the target templates. At least 2000 extended extension primers are produced from at least one of the target templates.

An average of at least 50 extended extension primers can be produced from each of the target templates. An average of at least 100 extended extension primers can be produced from each of the target templates. An average of at least 200 extended extension primers can be produced from each of the target templates. An average of at least 400 extended extension primers can be produced from each of the target templates. An average of at least 800 extended extension primers can be produced from each of the target templates. An average of at least 1000 extended extension primers can be produced from each of the target templates. An average of at least 2000 extended extension primers can be produced from each of the target templates.

In some forms, the method can further comprise detecting one or more of the extended extension primers. Each extended extension primer can correspond to one or more of the target templates. The target templates can correspond to nucleic acid sequences of interest. Detection of an extended extension primer can indicate detection of the nucleic acid sequence of interest to which a target template corresponding to the detected extended extension primer corresponds. Detection of the extended extension primers can be performed simultaneously with incubation of the extension primers and target templates. The extension primers can comprise a detection label, and the extended extension primers can be detected via the detection label. The extended extension primers can comprise a detection label, and the extended extension primers can be detected via the detection label. The extended extension primers can be detected via interaction of the extended extension primers with detection probes. The interaction can be a base pairing interaction. The interaction can be a hybridization interaction. The extended extension primers can be covalently coupled to the detection probes. The extended extension primers can be covalently coupled to the detection probes by ligation. The detection probes can be stem-loop probes. The extended extension primers can be covalently coupled to anchor probes, and covalent coupling of the extended extension primers can be facilitated by the interaction of the extended extension primers with the detection probes. The anchor probes can be associated with a substrate. The extended extension primers can be covalently coupled to the anchor probes by ligation. The detection probes each comprise an oligonucleotide can comprise modified nucleotides or an oligonucleotide analog, and the detection probes can have a higher melting temperature than an oligonucleotide consisting of unmodified nucleotides having the same nucleotide base composition as the oligonucleotide comprising modified nucleotides or the oligonucleotide analog. The detection probes can comprise peptide nucleic acid.

The melting temperature of the extended extension primers interacting with the detection probes can be at least 5° C. higher than the melting temperature of the extended extension primers interacting with the target templates. The melting temperature of the extended extension primers interacting with the detection probes can be at least 10° C. higher than the melting temperature of the extended extension primers interacting with the target templates. The melting temperature of the extended extension primers interacting with the detection probes can be at least 6° C. higher than the temperature at which the extension primers and target templates are incubated. The melting temperature of the extended extension primers interacting with the detection probes can be at least 11° C. higher than the temperature at which the extension primers and target templates are incubated. The melting temperature of the extended extension primers interacting with the detection probes is at least 20° C. higher than the temperature at which the extension primers and target templates are incubated.

The detection probes can each comprise a primer complement portion. The primer complement portion of the detection probes can each comprise an oligonucleotide comprising modified nucleotides or an oligonucleotide analog, and the primer complement portions of the detection probes can have a higher melting temperature than an oligonucleotide consisting of unmodified nucleotides having the same nucleotide base composition as the oligonucleotide comprising modified nucleotides or the oligonucleotide analog. The primer complement portion comprises peptide nucleic acid. The extended extension primers each comprise an extension portion, wherein the extension portion comprises nucleotides added to the extension primer by extension, wherein the extended extension primers each comprise an extended target complement portion and a non-target complement portion, wherein the extended target complement portion comprises the target complement portion and the extension portion. The detection probes can interact with both the extended target complement portion and the non-target complement portion of the extended extension primers.

The melting temperature of the extended extension primers interacting with the detection probes can be at least 5° C. higher than the melting temperature of the extended extension primers interacting with the target templates. The melting temperature of the extended extension primers interacting with the detection probes can be at least 10° C. higher than the melting temperature of the extended extension primers interacting with the target templates. The melting temperature of the extended extension primers interacting with the detection probes can be at least 6° C. higher than the temperature at which the extension primers and target templates are incubated. The melting temperature of the extended extension primers interacting with the detection probes can be at least 11° C. higher than the temperature at which the extension primers and target templates are incubated. The melting temperature of the extended extension primers interacting with the detection probes can be at least 20° C. higher than the temperature at which the extension primers and target templates are incubated.

The detection probes can be associated with a substrate. The detection probes can be attached to the substrate. The detection probes can be covalently coupled to the substrate. The detection probes can be indirectly associated with the substrate. The detection probes can be directly associated with the substrate. The substrate can be a single substrate structure, and all of the detection probes can be associated with a single substrate. The substrate can be a thin film, membranes, bead, microbead, bottle, dish, slide, fiber, optical fiber, woven fiber, chip, compact disk, shaped polymer, particles, or microparticle. The substrate can be comprised of a plurality of substrate structures. The substrate comprises a plurality of beads. The beads can be microbeads. The beads can be paramagnetic beads. The substrate can be acrylamide, cellulose, nitrocellulose, glass, gold, polystyrene, polyethylene vinyl acetate, polypropylene, polymethacrylate, polyethylene, polyethylene oxide, polysilicate, polycarbonate, teflon, fluorocarbon, nylon, silicon rubber, polyanhydride, polyglycolic acid, polylactic acid, polyorthoester, functionalized silane, polypropylfumerate, collagen, glycosaminoglycan, or polyamino acid.

A plurality of extension primers can be brought into contact with a plurality of target templates, and multiple extended extension primers can be produced from each of a plurality of the target templates. The extension primers and target templates can be incubated in the same reaction. The extension primers and target templates can be incubated in a plurality of different reactions. Each extension primer can be incubated with the target templates in a different reaction. At least two extension primers can be incubated with the target templates in a different reaction.

The target templates can each comprise a primer complement region, and the extension primers can each comprise a target complement portion. The target complement portion can be complementary to a primer complement region of one or more of the target templates, and the extension primers can comprise a set of extension primers. Each target complement portion of each extension primer in the set can have similar hybrid stability.

The extended extension primers can comprise a set of extended extension primers, and each extended extension primer in the set can have similar hybrid stability. The melting temperatures of each target complement portion of each extension primer in the set can be within 5° C. The melting temperatures of each target complement portion of each extension primer in the set can be within 3° C. The melting temperatures of each target complement portion of each extension primer in the set can be within 2° C. The extended extension primers can comprise a set of extended extension primers, and each extended extension primer in the set can have similar hybrid stability. The melting temperatures of each extended extension primer in the set can be within 7° C. The melting temperatures of each extended extension primer in the set can be within 5° C. The melting temperatures of each extended extension primer in the set can be within 3° C.

The extended extension primers can each comprise an extension portion. The extension portion can comprise nucleotides added to the extension primer by extension. The extended extension primers can each comprise an extended target complement portion. The extended target complement portion can comprise the target complement portion and the extension portion. The extended extension primers can comprise a set of extended extension primers, and the extended target complement portion of each extended extension primer in the set can have similar hybrid stability.

The melting temperature of the extended target complement region of each extended extension primer in the set can be within 7° C. The melting temperature of the extended target complement region of each extended extension primer in the set can be within 5° C. The melting temperature of the extended target complement region of each extended extension primer in the set can be within 3° C.

The extended extension primers each further can comprise a non-target complement region, and each extended extension primer in the set has similar hybrid stability.

The melting temperature of the extended extension primers can be at least 5° C. higher than the melting temperature of the extended target complement portion of the extended extension primers. The melting temperature of the extended extension primers can be at least 10° C. higher than the melting temperature of the extended target complement portion of the extended extension primers. The melting temperature of the extended extension primers can be at least 6° C. higher than the temperature at which the extension primers and target templates are incubated. The melting temperature of the extended extension primers can be at least 11° C. higher than the temperature at which the extension primers and target templates are incubated. The melting temperature of the extended extension primers can be at least 20° C. higher than the temperature at which the extension primers and target templates are incubated.

In some forms, the method can further comprise detecting one or more of the extended extension primers.

Each extended extension primer can correspond to a different target template. Each target template can correspond to a different nucleic acid sequence of interest. Detection of an extended extension primer can indicate detection of the nucleic acid sequence of interest to which the target template corresponding to the detected extended extension primer corresponds. Detection of a plurality of extended extension primers can indicate detection of the nucleic acid sequences of interest to which the target templates corresponding to the detected extended extension primers correspond. Detection of the extended extension primers can be performed simultaneously with incubation of the extension primers and target templates.

The extension primers can comprise a detection label. The detection label of each extension primer can be the same, and the extended extension primers can be detected via the detection label.

The extended extension primers can comprise a detection label. The detection label of each extended extension primer can be the same, and the extended extension primers can be detected via the detection label.

The extension primers can comprise a detection label. The detection label of each extension primer can be different, and the extended extension primers can be detected via the detection label.

The extended extension primers can comprise a detection label. The detection label of each extended extension primer can be different, and the extended extension primers are detected via the detection label.

A signal quotient can be calculated for one or more of the detected extended extension primers. The extended extension primers can be detected via detection labels. A signal quotient can be calculated for one or more of the detected extended extension primers using the detection labels. The extended extension primers can be detected via detection labels. The detection labels can generate signals, and a signal quotient can be calculated for one or more of the detected extended extension primers using the signals.

The extended extension primers can be detected via detection labels. The detection labels can generate signals. The intensity of the signal generated for each detected extended extension primer can be measured, and a signal quotient can be calculated for one or more of the detected extended extension primers using the intensity of the measured signals.

The signal quotient can be a median signal quotient. The median signal quotient of a given extended extension primer can be calculated by dividing the signal intensity generated for that extended extension primer by the median signal intensity generated for the detected extended extension primers.

The median signal quotient of a given extended extension primer can be calculated by dividing the signal intensity generated for that extended extension primer by the median signal intensity generated for all of the detected extended extension primers.

The median signal quotient of a given extended extension primer can be calculated by dividing the signal intensity generated for that extended extension primer by the median signal intensity generated for a subset of all of the detected extended extension primers.

The signal quotient can be a ratio of signal quotients, and the ratio of signal quotients of a given extended extension primer can be calculated by dividing the median signal quotient for that extended extension primer by the average signal quotient for that extended extension primer.

Each of the plurality of amplifications can be carried out in the same way.

The average signal quotient of a given extended extension primer can be calculated by dividing the average signal intensity generated for that extended extension primer in 10 or more amplifications by the average signal intensity generated for the detected extended extension primers in the 10 or more amplifications.

The average signal quotient of a given extended extension primer can be calculated by dividing the average signal intensity generated for that extended extension primer in 20 or more amplifications by the average signal intensity generated for the detected extended extension primers in the 20 or more amplifications.

The average signal quotient of a given extended extension primer can be calculated by dividing the average signal intensity generated for that extended extension primer in 50 or more amplifications by the average signal intensity generated for the detected extended extension primers in the 50 or more amplifications.

The average signal quotient of a given extended extension primer can be calculated by dividing the average signal intensity generated for that extended extension primer in all of the amplifications by the average signal intensity generated for the detected extended extension primers in all of the amplifications.

The average signal quotient of a given extended extension primer can be calculated by dividing the average signal intensity generated for that extended extension primer in a subset of all of the amplifications by the average signal intensity generated for the detected extended extension primers in a subset of all of the amplifications.

The extended extension primers can be detected via interaction of the extended extension primers with detection probes, wherein each extended extension primer corresponds to a different detection probe, wherein each extended extension primer can interact with the detection probe to which the extended extension primer corresponds.

The interaction can be a base pairing interaction. The interaction can be a hybridization interaction. The extended extension primers can be covalently coupled to the detection probes. The extended extension primers can be covalently coupled to the detection probes by ligation. The detection probes can be stem-loop probes.

The extended extension primers can be covalently coupled to anchor probes. Covalent coupling of the extended extension primers can be facilitated by the interaction of the extended extension primers with the detection probes, and each extended extension primer can correspond to a different anchor probe.

The anchor probes can be associated with a substrate. The extended extension primers can be covalently coupled to the anchor probes by ligation. The detection probes can comprise peptide nucleic acid.

The melting temperature of the extended extension primers can interact with the detection probes to which the extended extension primers correspond is at least 5° C. higher than the melting temperature of the extended extension primers interacting with the target templates to which the extended extension primers correspond.

The melting temperature of the extended extension primers can interact with the detection probes to which the extended extension primers correspond is at least 10° C. higher than the melting temperature of the extended extension primers interacting with the target templates to which the extended extension primers correspond.

The melting temperature of the extended extension primers can interact with the detection probes to which the extended extension primers correspond is at least 6° C. higher than the temperature at which the extension primers and target templates are incubated.

The melting temperature of the extended extension primers can interact with the detection probes to which the extended extension primers correspond is at least 11° C. higher than the temperature at which the extension primers and target templates are incubated.

The melting temperature of the extended extension primers can interact with the detection probes to which the extended extension primers correspond is at least 20° C. higher than the temperature at which the extension primers and target templates are incubated.

The detection probes can each comprise a primer complement region. The primer complement region can comprise peptide nucleic acid. The detection probes can be associated with a substrate. The detection probes can be attached to the substrate. The detection probes can be covalently coupled to the substrate. The detection probes can be indirectly associated with the substrate. The detection probes can be directly associated with the substrate. The substrate can be a single substrate structure, and all of the detection probes can be associated with a single substrate. The detection probes can be each associated with a different region of the substrate. The region of the substrate where an extended extension primer is detected can be indicative of the identity of the detected extended extension primer. The substrate can be a thin film, membranes, bead, microbead, bottle, dish, slide, fiber, optical fiber, woven fiber, chip, compact disk, shaped polymer, particles, or microparticle. The substrate can comprise a plurality of substrate structures. The detection probes can be each associated with a different substrate structure. The substrate structure on which an extended extension primer is detected can be indicative of the identity of the detected extended extension primer. The detection probes can be each associated with a different region of a substrate structure, a different substrate structure, or a different region of a different substrate structure. The substrate structure and region of the substrate structure where an extended extension primer is detected can be indicative of the identity of the detected extended extension primer.

The substrate can comprise a plurality of beads. The beads can be microbeads. The beads can be paramagnetic beads. The substrate can be acrylamide, cellulose, nitrocellulose, glass, gold, polystyrene, polyethylene vinyl acetate, polypropylene, polymethacrylate, polyethylene, polyethylene oxide, polysilicate, polycarbonate, teflon, fluorocarbon, nylon, silicon rubber, polyanhydride, polyglycolic acid, polylactic acid, polyorthoester, functionalized silane, polypropylfumerate, collagen, glycosaminoglycan, or polyamino acid.

The target templates each comprise a primer complement region, an extension region, and replication-terminating feature. The extension primers each comprise a target complement portion, wherein the target complement portion is complementary to a primer complement region of one or more of the target templates. The replication-terminating feature can be a 5′ end, one or more abasic nucleotides, or one or more derivatized nucleotides. The replication-terminating feature can be a 5′ end.

The target templates can correspond to nucleic acid molecules comprising nucleic acid sequences of interest, and the replication terminating feature of at least one of the target templates can be produced by cleavage of the nucleic acid molecule to which the target template corresponds. The cleavage can be accomplished using one or more restriction endonucleases, cleavase I, T4 endonuclease, endonuclease IV, one or more resolvases, or a combination. The nucleic acid molecule can be DNA containing deoxyuridine, and the cleavage can be accomplished by excising uracil residues in the nucleic acid molecule using uracil DNA glycosylase and cleaving the backbone of the nucleic acid molecule with endonuclease. The uracil residues can be excised in the presence of single stranded binding protein, and excision of uracil in loops of the nucleic acid molecule can be favored over excision of uracil in single-stranded regions of the nucleic acid molecule. The DNA containing deoxyuridine can be produced by replicating a nucleic acid molecule using one or more DNA primers containing one or more deoxyuridines. One or more mismatch probes can be hybridized to the nucleic acid molecule. There can be at least one mismatch between each mismatch probe and the nucleic acid molecule. The cleavage can be accomplished by using cleavase I, wherein cleavase I cleaves at the mismatches.

### Illustrations

A. Analysis of Sequences in Human Genomic DNA from Tissues.

The following is an illustration of a form of the disclosed method used to determine if there are abnormalities in gene dosage relative to the dosage in DNA from blood.

1. Genomic DNA is extracted from a tissue biopsy using standard methods. The DNA is digested with a type IIS restriction enzyme, which cleaves approximately every 512 bases, on the average.

2. An array of 96 extension primer sets is prepared for use in a microtiter well format, where each set comprises 32 different extension primers, where the extension primers in each set are designed according to the criteria required for thermodynamic equilibrium extension, as described herein, to target the 5′-termini of 32 different genomic loci of known sequence. As described elsewhere herein, design criteria include, for example, the melting temperature of the extension primers and extended extension primers relative to the temperature and extension reaction conditions. In a preferred embodiment, each primer is specific for a different sequence present at the 5′-end of a unique restriction fragment of the genomic DNA. In a simple embodiment, the total number of different extension primers is 96×32=3,072. In other embodiments, the number of extension primers used in a single set can comprise 64 different extension primers, for a total of 96×64=6,144 primers per microtiter plate. An example of an even larger array of extension primers would use a microtiter plate of 384 wells, with 64 primers per well, for a total of 384×64=24,576 primers. Embodiments with more than 64 primers per set can also be used. However, when the number of primers exceeds 64, the number of unwanted, or artifactual priming reactions can increase.

3. The extension primers include a fluorescein moiety at the 5′-end as a detection label.

4. Thermodynamic equilibrium extension is performed for 2 hours at 72° C., using ThenmoSequenase DNA polymerase. All extension primers in a set of 32 (or 64) primers are extended and released in solution, having incorporated at their 3-end a sequence of, for example, 5 to 11 bases, corresponding to the complement of the 5′-terminus of the restriction fragments of genomic DNA. The number of incorporated bases is generally determined by the design of the extension primers relative to the target sequences.

5. After completion of the thermodynamic equilibrium extension amplification, the material in each well is contacted with a small array of probes, with each array located within a single well of a microtiter dish. The array contains 32 detection probes, each detection probe designed specifically for capture of a specific extended extension primer produced from a primer in the set of 32 extension primers. If the set contains 64 primers, the array will likewise contain 64 detection probes.

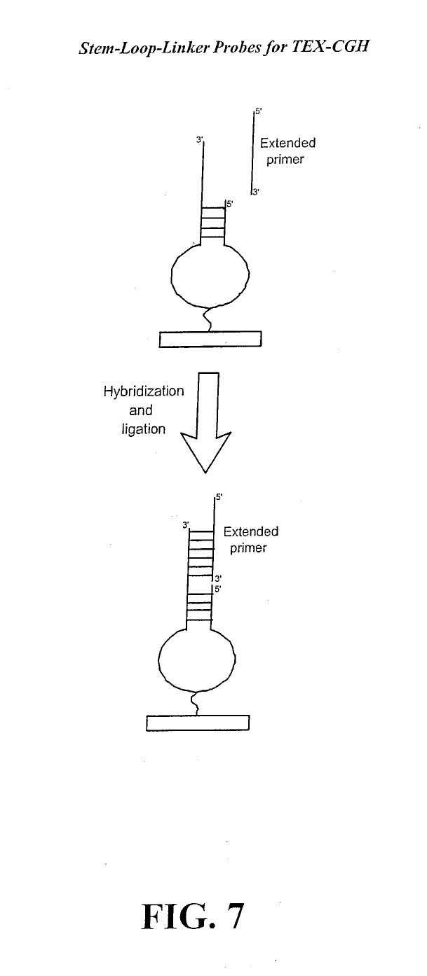

6. A preferred design for the capture step is the use of stem-loop probes as the detection probes. Stem-loop probes are described by Broude, N E, Woodward, K, Cavallo, R, Cantor, C R, and Englert, D 2001, nucleic Acids Research, 29:e92. Each stem-loop detection probe is designed to facilitate ligation of a specific extended extension primer to a phosphorylated 5′-end in the stem-loop detection probe, as illustrated in the diagram of FIG. 7. Sequences in the single stranded portion of the stem-loop detection probe (the primer complement portion of the detection probes) are complementary to sequences of the extended extension primer (the probe complement portion of the extended extension probes), including 5 to 11 bases of extended sequence (the extension portion of the extended extension primer), plus another 1 to 6 bases of the extended extension primer. Ligation is performed at using T4 DNA ligase, under the conditions described by Boude et al (2001). After ligation, the array is washed to remove unligated DNA.

Extension Primer (nucleotides 1 to 16 of SEQ ID NO:10):

cgacatacgaggatag-3′

Extended Extension Primer (extension portion in CAPS; nucleotides 1 to 26 of SEQ ID NO:10):

cgacatacgaggatagCAGACAGATG-3′

Stem-loop Detection Probe on Array Surface (nucleotides 27 to 65 of SEQ ID NO:10):

```
                            5′-pACTAGTCGTTA
                                        |||||||| T---array surface
3′-CCTATCGTCTGTCTAC TGATCAGCCAT
```

Annealing of Extended Extension Probe (nucleotides 1 to 26 SEQ ID NO:10 and nucleotides 27 to 65 of SEQ ID NO:10):

```
cgacatacgaggatagCAGACAGATGpACTAGTCGTTA
                    |||||||||||||||| |||||||| T---array surface
                    CCTATCGTCTGTCTAC TGATCAGCCAT
```

Ligation of Extended Extension Probe (SEQ ID NO:10):

```
cgacatacgaggatagCAGACAGATGACTAGTCGTTA
                    |||||||||||||||||||||||| T---array surface
                    CCTATCGTCTGTCTACTGATCAGCCAT
```

6A. Alternative sequence design of probes used when the polymerase adds a single untemplated base, most frequently an A, after the last templated nucleotide addition by the TEX reaction.

Extension Primer (nucleotides 1 to 16 of SEQ ID NO:11):

cgacatacgaggatag-3′

Extended Extension Primer (extension portion in CAPS; untemplated “A” addition shown BOLD; nucleotides 1 to 27 SEQ ID NO:11):

cgacatacgaggatagCAGACAGATGA-3′

Stem-loop Detection Probe on Array Surface, with additional base designed to pair with untemplated “A” (nucleotides 28 to 67 of SEQ ID NO:11):

5′-pACTAGTCGTTA
|||||||| T--array surface
3′-CCTATCGTCTGTCTACT TGATCAGCCAT

Annealing of Extended Extension Probe (nucleotides 1 to 27 SEQ ID NO:11 and nucleotides 28 to 67 of SEQ ID NO:11):

```
cgacatacgaggatagCAGACAGATGA-3′
cgacatacgaggatagCAGACAGATGApACTAGTCGTTA
                    ||||||||||||||||| |||||||| T--array surface
                    CCTATCGTCTGTCTACT TGATCAGCCAT
```

Ligation of Extended Extension Probe (SEQ ID NO:11):

```
cgacatacgaggatagCAGACAGATGAACTAGTCGTTA
                    ||||||||||||||||||||||||| T--array surface
                    CCTATCGTCTGTCTACTTGATCAGCCAT
```

7. Detection of ligated extended extension primers is performed by detection of fluorescent signal in a microarray scanner capable of reading microtiter dishes. (Alpha Innotech Corporation 14743 Catalina St. San Leandro, Calif. 94577).

8. Signals are processed (normalized) by calculating a Ratio of Signal Quotients (RSQ) where RSQ=Median Signal Quotient (MSQ)/Average Signal Quotient (ASQ). Median Signal Quotient is obtained by dividing the signal intensity of the extended extension primers bound at one specific probe address by the median of the signal intensities of all extended extension primers at all addresses. Average Signal Quotient is calculated as the average signal intensity of the extended extension primers bound at one specific probe address over a number of calibration assays divided by the average signal intensity of all extended extension primers bound at all addresses for all of the calibration assays. The calibration assays generally will be separate assays performed using the same label and same reagents using DNA from normal individuals. Average Signal Quotient represents the relative signal intensity of extended extension primers at a probe array element, compared to the total ensemble of extended extension primers at all of the probe array elements, in a calibration assay. The average of many measurements is calculated in order to account for possible variations in results.

Generally, the more calibration assays performed, the better the results. For example, 10 or more or 16 or more calibration assays woulf be a useful number. Experimental error decreases with the square root of the number of observations. Thus, for 9 observations the observed error is expected to be three times lower than for a single observation. For 16 observations, the observed error is expected to be four times lower than for a single observation. The more observations used for this calculation, the lower the expected error. Thus, performing more experiments for use in calculating the ASQ can provide better results.

9. The Ratio of Signal Quotients (RSQ) can then be plotted to show gains and losses of alleles across the genome. Examples of such plots are shown in FIGS. 5 and 6.

B. Detection of Hepatitis C virus Using Thermodynamic Equilibrium Extension of Primers and Rolling Circle Amplification

The following illustrates detection of specific nucleic acid sequences in hepatitis C virus using a form of the disclosed method.

1. Nucleic Acid Sequence Preparation

Add 200 μl of 6M guanidinium thiocianate in 0.1M Tris-HCl, pH 7.4 (GTC) to 100 μl of whole blood or serum.

Add 50 μl of 3-5% Aerosil A-300 in 4M GTC (4M guanidinium thiocianate in 0.1M Tris-HCl, pH 7.4), mix intensively 5 second.

Spin 2 minutes at 10,000-15,000 rpm.

Transfer supernatant into new tube.

Add 10 μl of streptavidin-paramagnetic beads (SA-PMB), coated with chimera 2′-O-methyl RNA-DNA (biotin-(C4)-TTTTacctcccggggcactcgcaagcaccCTATCAGGCA; SEQ ID NO:1), mix, incubate 10-15 minutes. Uppercase nucleotides are deoxyribonucleotides and lowercase nucleotides are 2′-O-methyl ribonucleotides.

Separate PMB, using magnet.

Wash 2 times with 30 μl 4M GTC.

Wash 2 times with 300 μl 6×SSPE (SSC).

Wash 1 time with 300 μl 10 mM Tris-HCl, pH 8.0.

Add 20 μl mixture for reverse transcription (1× buffer for RT, 200 mM of each dNTP, 1U AMV reverse transcriptase), incubate at 42° C. for 30-40 minutes.

2. Target Template Preparation

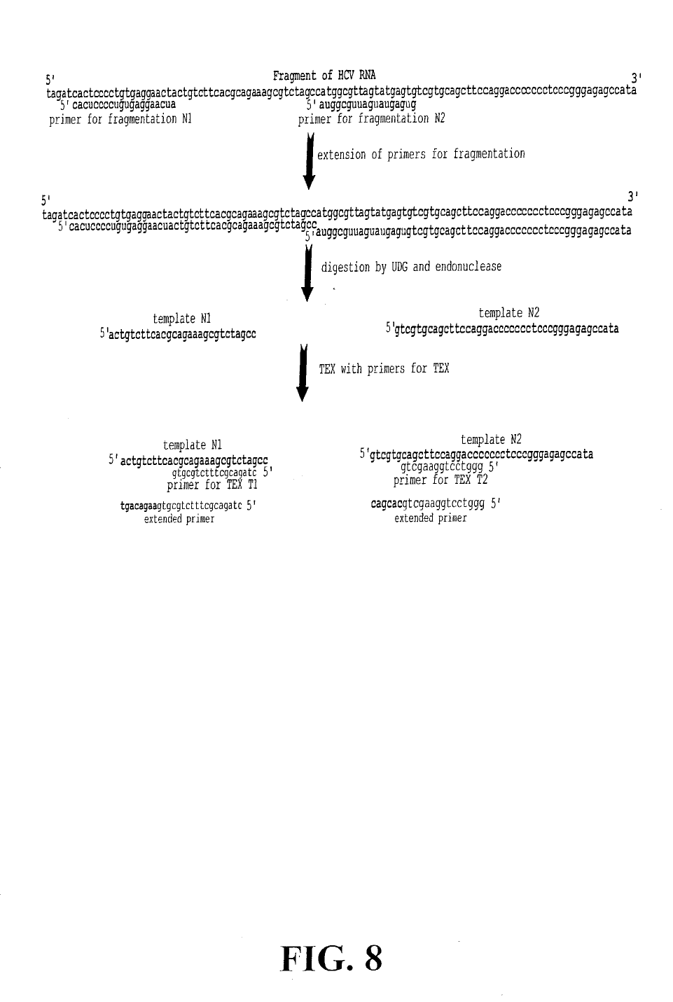

Capture PMB by magnet, add fragmentation primers N1 and N2 (FIG. 8), to a 1 μM final concentration. These primers are complementary to sequences in hepatitis C virus.

| | | |
|---|---|---|
| Fragmentation primer N1: | cacUccccUgUgaggaacUa | (nucleotides 1 to 20 of SEQ ID NO:2) |
| Fragmentation primer N2: | aUggcgUUagUaUgagUg | (nucleotides 1 to 18 of SEQ ID NO:3) |

Incubate at 94° C. for 3-5 minutes, at 37° C. for 1 minute, add 0.5U of T4 DNA polymerase, incubate at 37° C. for 3 minute.

3. Amplification by Thermodynamic Equilibrium Extension of Primers

Capture PMB by magnet, remove super, add 20 μl mixture for TEX without polymerase (1× ThermoPol buffer, 200 mM of each dNTP, 1 μM of each extension primers T1 and T2 (FIG. 8)).

| | | |
|---|---|---|
| Extension Primer T1: | ctagacgctttctgcgtg | (nucleotides 1 to 18 of SEQ ID NO:4) |
| Extension Primer T2: | gggtcctggaagctg | (nucleotides 1 to 15 of SEQ ID NO:5) |

Incubate sample at 94° C. for 1 minute, capture PMB, remove supernatant in new tube.

Add polymerase for TEX (DeepVent(exo−), for example), incubate sample at 70-72° C. for 1-3 hours on microarray slide.

Wash slide 2 times at 50° C. for 5 minutes in 0.4×SSC.

Detect extended product after hybridization by rolling circle amplification.

C. Detection of Hepatitis C Virus Using Thermodynamic Equilibrium Extension of Primers and Direct Detection

The following illustrates detection of specific nucleic acid sequences in hepatitis C virus using a form of the disclosed method.

1. Nucleic Acid Sequence Preparation

Add 200 μl of 6M guanidinium thiocianate in 0.1M Tris-HCl, pH 7.4 (GTC) to 100 μl of whole blood or serum.

Add 50 μl of 3-5% Aerosil A-300 in 4M GTC (4M guanidinium thiocianate in 0.1M Tris-HCl, pH 7.4), mix intensively 5 second.

Spin 2 minutes at 10,000-15,000 rpm.

Transfer supernatant into new tube.

Add 10 μl of streptavidin-paramagnetic beads (SA-PMB), coated with chimera 2′-O-methyl RNA-DNA (biotin-(C4)-TTTTacctcccggggcactcgcaagcaccCTATCAGGCA; SEQ ID NO:1), mix, incubate 10-15 minutes. Uppercase nucleotides are deoxyribonucleotides and lowercase nucleotides are 2′-O-methyl ribonucleotides.

Separate PMB, using magnet.

Wash 2 times with 30 μl 4M GTC.

Wash 2 times with 300 μl 6×SSPE (SSC).

Wash 1 time with 300 μl 10 mM Tris-HCl, pH 8.0.

Add 20 μl mixture for reverse transcription (1× buffer for RT, 200 mM of each dNTP, 1U AMV reverse transcriptase), incubate at 42° C. for 30-40 minutes.

2. Target Template Preparation

Capture PMB by magnet, add fragmentation primers N1 and N2 (FIG. 8), to a 1 μM final concentration. These primers are complementary to sequences in hepatitis C virus.

| | | |
|---|---|---|
| Fragmentation primer N1: | cacUccccUgUgaggaacUa | (nucleotides 1 to 20 of SEQ ID NO:2) |
| Fragmentation primer N2: | aUggcgUUagUaUgagUg | (nucleotides 1 to 18 of SEQ ID NO:3) |

Incubate at 94° C. for 3-5 minutes, at 37° C. for 1 minute, add 0.5U of T4 DNA polymerase, incubate at 37° C. for 3 minute.

3. Amplification by Thermodynamic Equilibrium Extension of Primers

Capture PMB by magnet, remove super, add 20 μl mixture for TEX without polymerase (1× ThermoPol buffer, 200 mM of each dNTP, 1 μM of each extension primers T1 and T2 (FIG. 8)), where the extension primers are labeled with Cy3 and Cy5, respectively.

| | | |
|---|---|---|
| Extension Primer T1: | Cy3-ctagacgctttctgcgtg | (nucleotides 1 to 18 of SEQ ID NO:4) |
| Extension Primer T2: | Cy5-gggtcctggaagctg | (nucleotides 1 to 15 of SEQ ID NO:5) |

Incubate sample at 94° C. for 1 minute, capture PMB, remove supernatant in new

Add polymerase for TEX (DeepVent(exo−), for example), incubate sample at 70-72° C. for 1-3 hours on microarray slide containing capture probes complementary to the

Wash slide 2 times at 50° C for 5 minutes in 0.4×SSC.

Detect extended product with a fluorescence scanner after hybridization on the glass slide with capture probes.

### EXAMPLE

### A. Example: Thermodynamic Equilibrium Extension of Primers

This following describes an example of one form of the disclosed method and compares different means of termination of primer extension. In the reaction, catalyzed by DNA polymerase, a small portion of a DNA template is copied multiple times by sequential primer binding, extension, and dissociation events. An extension primer, present in large excess relative to a target DNA sequence (the target template), is incubated above its melting temperature, and allowed to extend just a few bases, before a forced termination event occurs. To achieve specific termination of primer extension, different methods were tested: termination by dideoxynucleoside triphosphates, termination at the 5′ end of a template DNA. The last three methods of termination all involve a replication terminating feature. Dideoxy termination was the least effective of these methods, and termination at a 5′ end in the template was the most effective. Thermodynamic equilibrium extension (TEX) reactions generated between 500 and 1000 copies of a DNA target, and could be performed primers could be detected with high specificity using microarrays containing immobilized PNA or LNA detection probes. Notably, it was possible to perform the TEX directly on the surface the microarray, where primer extension and DNA:PNA hybridization took place concurrently with amplification. The PNA microarray was capable of discriminating a single base change on the extended extension primer sequence. Using primers labeled with a single fluorescein residue at the 5′-end, DNA targets present at a concentration of 0.4 pM (0.8 attommoles in 20 μl) were detected. The disclosed TEX amplification method, coupled viral genome quasi-species.

1. Materials And Methods

### i. Oligonucleotide Synthesis, Microchip Manufacturing

Oligonucleotides were obtained from the Yale Critical Technologies facility. Peptide nucleic acids (Nielsen, P. E. (1999) An introduction to PNA. In: “Peptide Nucleic Acids:Protocols and Application”) were purchased from Applied Biosystems, Framingham Mass., and locked nucleic acids (LNA) (Ørum, H., Jakobsen, M. H., Koch, T., Vuust, J. and Borre, M. B. (1999) Detection of the Factor V Leiden Mutation by Direct Allele-specific Hybridization of PCR Amplicons to Photoimmobilized Locked Nucleic Acids. Clin Chem., 45:1898-1905) were purchased from Proligo, Inc., Boulder, Colo. All modified primers were purified by gel purification or by reverse phase HPLC.

Polyacrylamide coated microchips were manufactured as described in Gerry, N. P., Witowski, N. E., Day, J., Hammer, R. P., Barany, G. and Barany, F. (1999) Universal DNA microarray method for multiplex detection of low abundance point mutations. J. Mol. Biol., 292:251-262. For manual spotting, 0.2 μl aliquots were taken from stock solutions (5 μM LNA in 150 mM sodium phosphate (pH 8.5) or 5 μM PNA in 30 mM Hepes (pH 7.0) with 12.5% of N-methylpyrrolidone), and deposited onto the preactivated polymeric surfaces. The resulting arrays were incubated overnight in humidified chamber. Following spotting, uncoupled probes were removed from the polymer surfaces by washing the slides in blocking Solution (0.1 M Tris-HCl, pH 9.0, 50 mM ethanolamine, 0.1% SDS) for 30 minutes at room temperature, rinsing twice with water, washing in 4×SSC/0.1% SDS for 30 minutes at room temperature, rinsing thrice with water and drying. The arrays were stored at 25° C. in slide boxes until needed.

ii. ssDNA Preparation

A 201nt long fragment of the 5′-UTR region of Hepatitis C virus (HCV) was prepared by PCR amplification from plasmid p35.2a that contains the fragment of HCV genome.

Aliquots of 50 μl of reaction mixture contained 50 ng of plasmid DNA, 1 μM of each primers (forward1—5′-gccatggcgttagtatgagtgtcgt-3′ (SEQ ID NO:7), reverse1—5′-aaggcctttcgcgacccaacactac-3′ (SEQ ID NO:8)), 0.2 mM of each dNTP (New England Biolab, USA) and 1 U Taq DNA polmerase in 1×PCR buffer (Fermentas, Lithuania). Amplification was carried out for 30 cycles of 95° C. of 30 s, 64° C. for 30 s and 72° C. for 30 s. PCR fragment was purified from the unicorporated dNTPs and the primers with microcentrifuge GF/F filters (Whatman, UK) (Gribanov, P. G., Shcherbakov, A. V., Perevozchikova, N. A., Gusev, A. A. (1996) Use of aerosol A-300 and GF/F (GF/C) filters for purifying fragments of DNA, plasmid DNA and RNA. Biokhimiia (Moscow), 61;1064-1070). Purified DNA was used for ssDNA generation by PCR. Aliquots of 50 μl of reaction mixture contained 100-200ng of purified DNA, 1 μM of primer—forward1 or reverse1,—0.2 mM of each dNTP (New England Biolab, USA) and 1 U Taq DNA polymerase in 1×PCR buffer (Fermentas, Lithuania). Amplification was carried out for 30 cycles of 95° C. of 20 s, 64° C. for 20 s and 72° C. for 20 s. Single-stranded PCR fragment was purified from the unicorporated dNTPs and primers by microcentrifugation on GF/F filters, as follows: 450 μl of 4 M Guanidine Thiocyanate in 0.1 M Tris-HCl (pH 7.4) (GTC) and 50 μl of 6% Aerosil A-300 (Serva, German) in 4 M GTC were added to 50 μl of reaction mixture. After brief shaking for 5-10 s and incubation for 1-2 minutes, Aerosil with adsorbed dsDNA was precipitated by centrifugation at 10000 rpm for 2-3 minutes. The supernatant was blended with equal volume of ethanol and was placed on a GF/F microcentrifuge filter. After washing once with 400 μl of 2M GTC in 50% ethanol and three times with 400 μl of 80% ethanol, the ssDNA was eluted from microcentrifuge filter with 50 μl of water.

iii. Multiprimer Fragmentation of cDNA

Multiprimer fragmentation of HCV cDNA was carried out as follows. A 247bp long fragment of the 5′-UTR region of Hepatitis C virus (HCV) was prepared by PCR amplification from plasmid p35.2a that contains the fragment of HCV genome. Aliquots of 50 μl of reaction mixture contained 50 ng of plasmid DNA, 1 μM of each primers (forward1 and reverse2—5′-biotin-(C4)-TTTTacctcccggggcactcgcaagcaccCTATCAGGCA-3′ (SEQ ID NO:9; capital letters—DNA, lowercase letters—O-Methyl-RNA)), 0.2 mM of each dNTP (New England Biolabs, USA) and 1 U Taq DNA polymerase in 1×PCR buffer (Fermentas, Lithuania). Amplification was carried out for 30 cycles of 95° C. of 30 s, 64° C. for 30 s and 72° C. for 40 s. The PCR amplicons were purified from the unincorporated P. G., Shcherbakov, A. V., Perevozchikova, N. A., Gusev, A. A. (1996) Use of aerosol A-300 and GF/F (GF/C) filters for purifying fragments of DNA, plasmid DNA and RNA. Biokhimiia (Moscow), 61; 1064-1070). After elution from the microcentrifuge filter with 50 μl of water, the PCR amplicons were adsorbed on paramagnetic beads (250 μg)(Dynal) two consecutive incubations with 100 mM NaOH for 2 minutes each, followed by washing with water. Finally, the beads were resuspended in 50 μl of water.

Paramagnetic beads (5 μl) with adsorbed single-stranded cDNA were resuspended in mixture contained 1 μM of each primer for fragmentation (with deoxyuridine in place of deoxythymidine) (primer1—5′-uggucugcggaaccggug-3′ (SEQ ID NO:12), primer2—5′-gcccccgcgagacugcua-3′ (SEQ ID NO:13), primer3 -5′acgaccggguccuuucut-3′ (SEQ ID NO:14)) in 1×T4 DNA polymerase buffer (New England Biolabs, USA) (40 μl). After a brief denaturation at 94° C. for 1 minute, the mixture was incubated at 37° C. for 1 minute, 0.5U T4 DNA polymerase (New England Biolabs, USA) was added, and the mixture was incubated at 37° C for 3-5 minutes. The resulting extended primers were used as target temple in reactions where extension is terminated at or near a deoxyuridine residue. For preparation of cDNA fragments with abasic sites or with removed primer part (that is, with a 5′ end generated next to a deoxyuridine residue), 3 U of uracil DNA glycosylase (Epicentre Technologies, USA) or 3 U of uracil DNA glycosylase together with 2U of T4 endonuclease V (Epicentre Technologies, USA), respectively, were added to mixture after 3-5 minutes incubation with T4 DNA polymerase with subsequent incubation at 37° C. for 30-60 minutes. Treatment with uracil DNA glycosylase removes the uracil base portion of the uridine nucleotide residues, making them abasic. Treatment of such abasic nucleotide residues with T4 endonuclease V cleaves the cDNA fragments at the site of the abasic nucleotide residue. The resulting “abasic” primers and fragmented primers were used as target temple in reactions where extension is terminated at an abasic nucleotide or at the 5′ end of the fragment, respectively. Upon completion of the reaction, beads were washed by 30 μl of 1× Thermopol buffer (New England Biolabs, USA). The beads were resuspended in 25 μl of 1× Thermopol buffer and cDNA fragments were eluted from the first strand cDNA by denaturation at 94° C. for 1 minute and with subsequent separation using paramagnetic beads.

iv. Thermodynamic Equilibrium Extension

Termination by ddNTP in tubes. Each 20 μl of reaction mixture contained target templates (0.4-4 nM synthetic deoxyoligonucleotide or HCV cDNA), 0.1-1 mM of one of four dideoxynucleotide triphosphates, 2 mM of three other deoxynucleotide triphosphates, 2 U of DeepVent(exo−) DNA polymerase (NEB) or 2 U of Vent(exo−) DNA polymerase (NEB) in 1× Thermopol buffer (NEB); or 3-30 U ThermoSequenase (Amersham Pharmacia Biotech, USA) in 1× Thermosequenase buffer (Amersham Pharmacia Botech, USA); or 1 U Taq polymerase (PE Biosystems, USA) in 1×PCR buffer (Fermentas, Lithuania). Sample was denatured at 95° C. for 1 minute before incubation at a constant temperature in the range of 60-80° C. during 1-3 hours. Different reaction temperatures were used for different reactions. The temperature of each reaction was kept constant. Upon completion of the reaction, 20 μl of STOP solution (50% formamide with 10 mM EDTA and bromphenol blue) was added and 10 μl of sample was analyzed by electrophoresis in 15% PAAG with 8M urea. The gel after electrophoresis was stained by SybrGreen II (Molecular Probes) for 15 minutes and analyzed by ImageAnalyzer 2000 using software provided with instrument. The degree of amplification was determined as ratio of the amount of the extended products to the amount of template.

Termination at 5′ end of template, at abasic site or on deoxyuridine in tubes. Conditions for termination at 5′ end of template contained 2 mM each of all four deoxynucleotide triphosphates.

Hybridization of extended products to LNA-PNA arrays. Arrays, covered with EasiSeal chamber (Hybaid, UK) for in situ PCR, were pre-incubated for 15 minutes at 37° C. in 2×SSC/0.1% BSA in humidified chamber. 20×SSC buffer were added to samples after extension to produce a final buffer concentration of 2×SSC. EasiSeal frame was filled with samples and the arrays were placed in humidified chambers and incubated for one hour at 40-70° C. Following hybridization, the arrays were washed twice for 5 minutes in 0.4×SSC at 50° C. (for 14 mer PNA and LNA) or at 63° C. (for 17 mer PNA). Fluorescent signals were measured using a microarray scanner (ArrayWoRx, Applied Precision, USA).

Termination at 5′ end of template on microarray. Twenty-five μl of a reaction mixture contained templates, 2 mM each deoxynucleotide triphosphate, 3-30 U ThermoSequenase in 1× Thermosequenase buffer (Amersham) or 2U DeepVent(exo−) DNA polymerase in 1× Thermopol buffer (NEB). The reaction mixture was placed on the microchips, covered with EasiSeal chamber (Hybaid) for in situ PCR and heated at 94° C. for 1 minute before incubation at 70° C. for 1-3 hours. Upon completion of the reaction, the microchips were washed twice in 0.4×SSC at 50° C. and once in 2×SSC/0.05% Tween20 at room temperature. Twenty-five μl of the mixture with avidin (0.08 μM avidin in 2×SSC/0.05% Tween20) were applied to each microchip after washing in 2×SSC and incubated at 37° C. for 30 minutes in humid chamber, and then slides were washed twice in 2×SSC/0.05% Tween20 at 37° C. for 15 minutes. Twenty-five μl of the mixture with FITC-labeled biotin (0.2 μM biotin in 2×SSC/0.05% Tween20) were applied to each microchip and incubated at 37° C. for 30 minutes in humid chamber, and then slides were washed twice in 2×SSC/0.05% Tween20 at 37° C. for 15 minutes and spin-dried. Arrays were imaged using a microarray scanner (ArrayWoRx, Applied Precision). The spatial resolution of scans was 10 μm per pixel. The resulting images were analyzed using ArrayWoRx software provided with the instrument or using ScanAlyze2 software developed by Michael Eisen (http://rana.stanford.edu/software).

2. Results

i. Thermodynamic Equilibrium Extension Using Different Templates

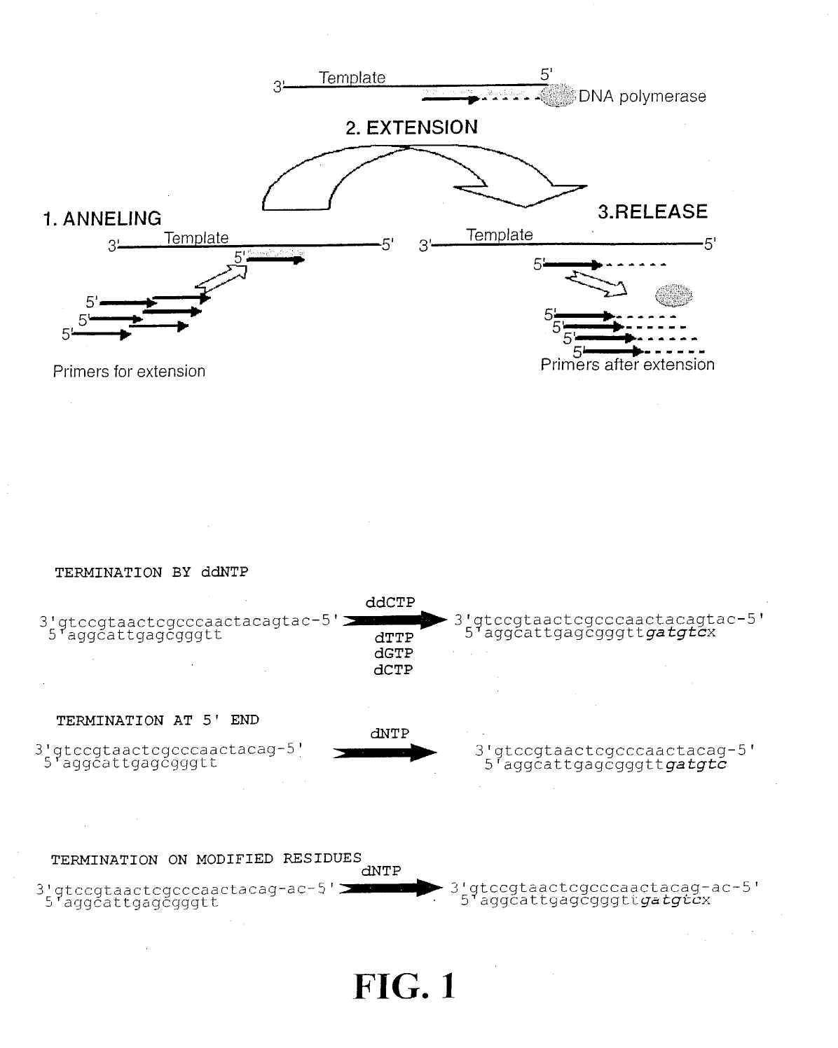

TEX was carried out at a temperature above the melting temperature of the duplex formed by target DNA and the extension primer. Under these conditions, the system is in dynamic equilibrium between the duplex and its dissociated state and the same DNA template can produce many primer extension products. As long as the primer extension is limited to no more than 10-12 bases, the stability of the extended duplex remains low enough to permit frequent dissociation events. Several different methods for the specific termination of primer extension were explored (FIG. 1: (1) termination by dideoxynucleotide triphosphates; (2) termination at the 5′ end of the template; (3) termination at abasic sites; (4) termination induced by deoxyuridine residues in the DNA template.

Primer extensions were carried out with either: 1) a synthetic 38 nt long DNA template (GACTGCTAGCCGAGTAGTGTTGGGTCGCGAAAGGCCTTGT-C3 Spacer (SEQ ID NO:6)) or; 2) cDNA derived from the 5′-UTR part of the of the Hepatitis C virus (HCV) genomic RNA. Different sets of primers were designed for synthetic templates, or for different sequences in the cDNA. Different enzymes (Vent(exo⁻), DeepVent(exo⁻), Taq polymerase, TheimoSequenase) were utilized to determine the efficiency of TEX amplification and the optimal method of specific termination of primer extension.

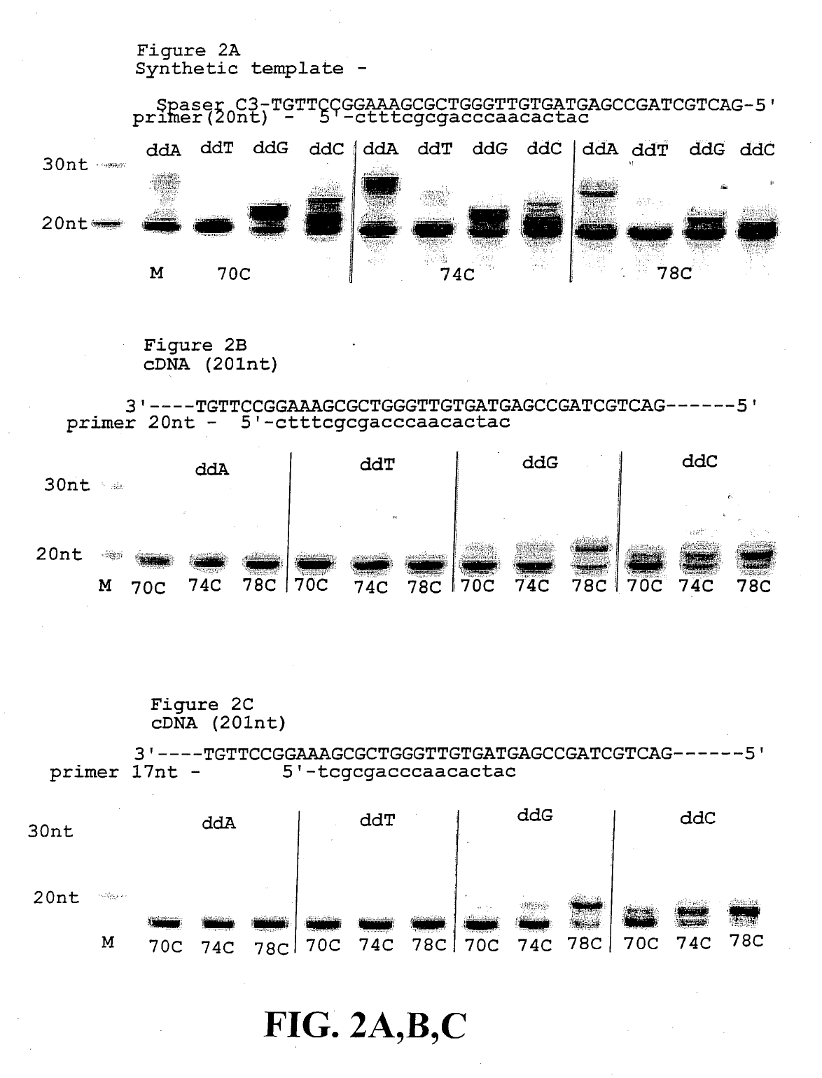

The best results involving dideoxynucleotide triphosphate termination were obtained using DeepVent(exo⁻) and ThermoSequenase. The highest amplification yields were approximately 300-fold during 2 hours of incubation, and the optimum temperature of extension was within the range of 68-74° C., depending on the primers used. The amplification obtained with short extension (less than 9-12nt) is weakly correlated with the melting temperature of primers and extended products and depends on temperature, template sequence and length, and primer concentration. As an example of how the length of template affects on extension, FIG. 2 shows the extension by DeepVent(exo⁻) of 17nt (T_{d}=59.1° C.) and 20nt (T_{d}=64.9° C.) primers on the synthetic template and on cDNA of HCV at different temperatures, with termination by different ddNTPs. Although the primer extension was carried out with the same primers (20nt, FIG. 2A and 2B), the amount and length of products differ for both of these templates. The cause of ineffective amplification on cDNA and non-specific termination by DeepVent(exo⁻) is not a function of the purity of the template, because other primers generate good levels of amplification on the same template.

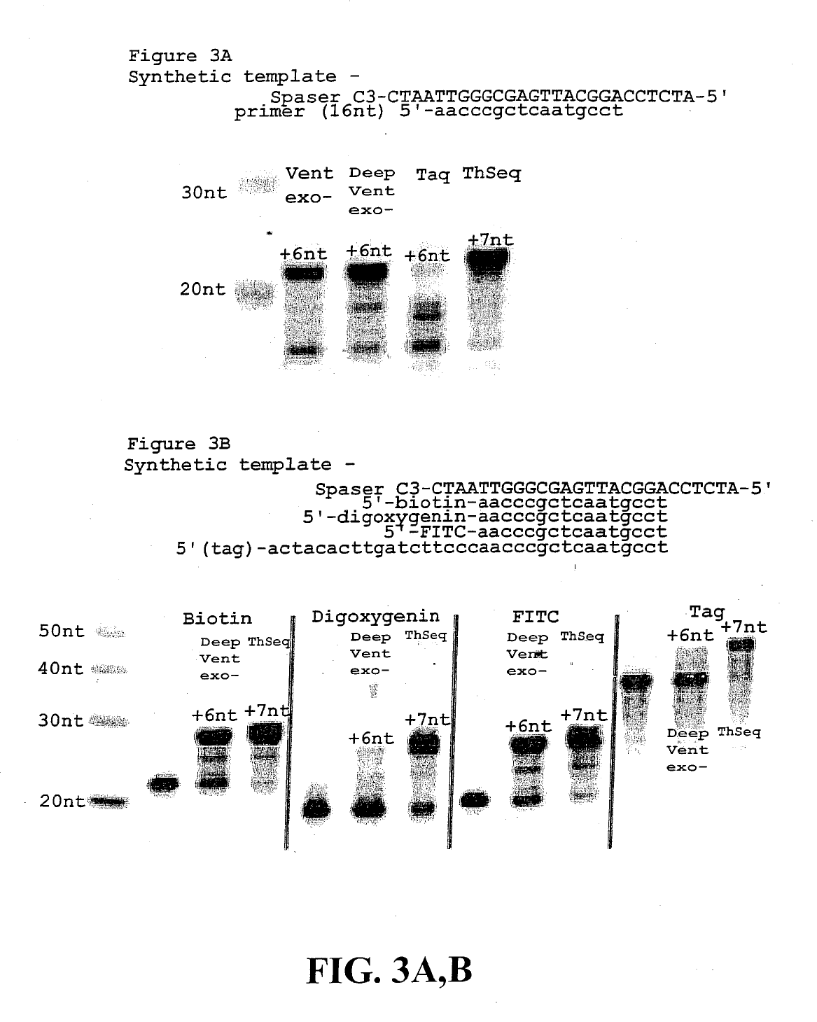

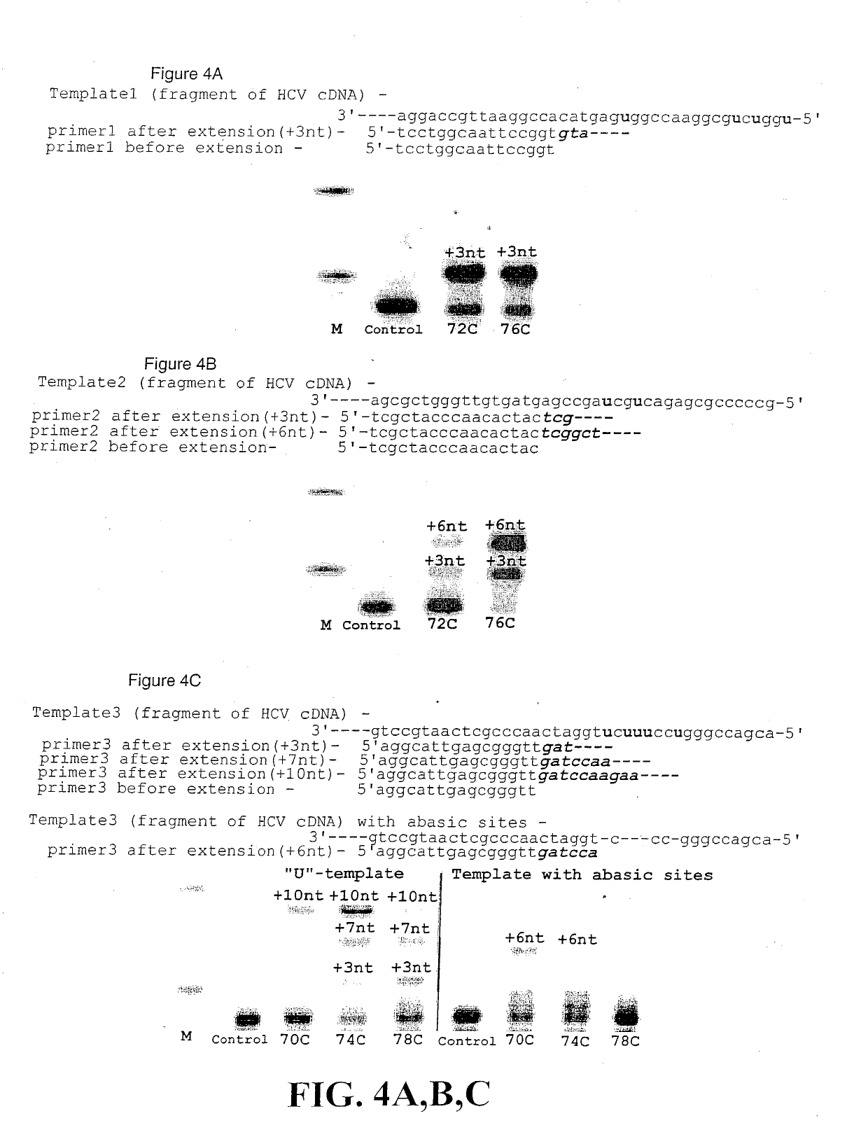

Amplifications were performed to demonstrate termination of extension by three different replication terminating features: the 5′ end of a template, abasic sites, and deoxyuridine residues. The amplifications were carried out with fragmented cDNA of the Hepatitis C virus (HCV) 5′-UTR part of the genome RNA. The cDNA was fragmented using T4 DNA polymerase, UDG and T4 endonuclease V as described above. The highest yield of TEX with termination at the 5′ end of template was obtained using DeepVent(exo⁻) and ThermoSequenase. The amplification yield ranged between 500 and 700-fold in a 2-hour incubation. It notable that ThermoSequenase adds one additional base to the extended primer (FIG. 3). The optimum temperature of extension was within the range of 68-72° C. for different primers. Since DeepVent(exo⁻) DNA polymerase and ThermoSequenase showed the best level of amplification by TEX, the influence of using extension primers that have different moieties at the 5′ end was determined. Digoxygenin, biotin, fluorescein, as well as a non-hybridizing non-specific sequence tag of 17nt on 5′ end of the primer were tested (FIG. 4). ThermoSequenase demonstrated excellent tolerance for all these modified primers, whereas DeepVent(exo⁻) DNA polymerase showed lower levels of amplification in two cases (fluorescein and 5′-DNA tag). The yield of TEX by DeepVent(exo⁻) DNA polymerase and ThermoSequenase are similar, when the extension was terminated at an abasic site and at 5′ end of template.

Unexpectedly, highly effective specific termination of extension by DeepVent(exo⁻) DNA polymerase was observed for templates containing deoxyuridine (FIG. 4). Chain elongation is terminated four bases away from (that is, prior to) deoxyuridine and the next deoxynucleotide of a template (FIG. 4). The highest primer extension yield was observed when the distance between the 3′ end of primer and deoxyuridine on template was not more than 6 bases (FIG. 4). In this case, the extended part of primers was only 3 nucleotides. If the template contains two (or more) deoxyuridines, and the distance between deoxyuridines is not more than three bases, amplification of alternative extended products is observed.

ii. TEX on Microarrays

For optimization of TEX on microarray, three different TEX amplifications with termination at the 5′ end of template were carried out with synthetic templates using microchips containing three immobilized PNA probes (Table 3).

Extension of FITC-labeled extension primer on microchip at 70° C. for 1 hour;

Extension of FITC-labeled extension primer on microchip at 70° C. for 1 hour, followed by incubation at 40-50° C. for 1 hour;

Extension of FITC-labeled extension primer in tubes at 70° C. for 1 hour, followed by hybridization on microchip at 40-50° C. for 1 hour.

In these reactions different amounts of templates (4 fM to 4 nM) were used for amplification. The limit of detection (40-400 fM of template) was obtained when the extension was carried out in test tubes, and the extended products were subsequently detected by hybridization on microchip at 40° C.

TABLE 3
Sequences of PNA probes and templates with primers for extension
used in this example.

Sequences

| | |
|---|---|
| primer1 | 5′biotin-aacccgctcaatgcct |
| | (nucleotides 19-34 of SEQ ID NO:23) |
| Template N1 | Spaser C3-CTAATTGGGCGAGTTACGGACCTCTA-5′ |
| | (SEQ ID NO:21) |
| PNA N1 | gttacggacctcta-O-L-L-5′ |
| | (nucleotides 1-14 of SEQ ID NO:21) |
| Extended primer1 | 5′biotin-aacccgctcaatgcctagagat |
| | (nucleotides 19-40 of SEQ ID NO:23) |
| Template M1 | Spaser C3-CTAATTGGGCGAGTTACGGATCTCTA-5′ |
| | (SEQ ID NO:22) |
| PNA M1 | gttacggatctcta-O-L-L-5′ |
| | (nucleotides 1-14 of SEQ ID NO:22) |
| Extended primer1 | 5′biotin-aacccgctcaatgcctagagat |
| | (SEQ ID NO:36) |
| primer2 | 5′biotin-gagagccatagtggtctg |
| | (nucleotides 1-18 of SEQ ID NO:34) |
| Template N2 | Spaser C3-CCCTCTCGGTATCACCAGACGCCTTG-5′ |
| | (SEQ ID NO:32) |
| PNA N2 | tcaccagacgccttg-O-L-L-5′ |
| | (nucleotides 1-15 of SEQ ID NO:32) |
| Extended primer2 | 5′biotin-gagagccatagtggtctgcggaac |
| | (SEQ ID NO:34) |
| Template M2 | Spaser C3-CCCTCTCGGTATCACCAGACCCCTTG-5′ |
| | (SEQ ID NO:33) |
| PNA M2 | tcaccagaccccttg-O-L-L-5′ |
| | (nucleotides 1-15 of SEQ ID NO:33) |
| Extended primer2 | 5′biotin-gagagccatagtggtctggggaac |
| | (SEQ ID NO:35) |
| Primer3 | 5′biotin-cccaacactactcggct |
| | (nucleotides 1-17 of SEQ ID NO:37) |
| Template N3 | Spaser C3-TGTTCCGGAAAGCGCTGGGTTGTGATGAGCCGATCGTCAG-5′ |
| | (SEQ ID NO:18) |
| PNA N3 | gagccgatcgtcag-O-L-L-5′ |
| | (nucleotides 1-14 of SEQ ID NO:18) |
| Extended primer3 | 5′biotin-cccaacactactcggctagcagtc |
| | (SEQ ID NO:37) |
| Template M3 | Spaser C3-TGTTCCGGAAAGCGCTGGGTTGTGATGAGCCGACCGTCAG-5′ |
| | (SEQ ID NO:19) |
| PNA M3 | gagccgaccgtcag-O-L-L-5′ |
| | (nucleotides 1-14 of SEQ ID NO:19) |
| Extended primer3 | 5′biotin-cccaacactactcggctggcagtc |
| | (SEQ ID NO:31) |

Six different synthetic templates were used in a multiprimer TEX&Hyb (isothermal equilibrium extension with simultaneous hybridization) on a PNA microarray. The microchip contained three or six PNA probes specific for each of the different extended products. The extended products (N1, N2, N3, M1, M2, M3) are fully matched to N1, N2, N3, M1, M2, M3 PNA probes, respectively, and extended products N1 and M1, N2 and M2, N3 and M3 differ from each other only in one nucleotide. TEX was carried out on a microchip for three different combinations of templates (4 nM of each). Positive fluorescence signals demonstrated that the TEX assay is specific, although the N2 and N3 PNA probes showed an unexpected duplex stability with products M2 and M3 at these conditions. Addition of the chaotropic salt guanidine thiocyanate (1M) (22) improved the specificity of the hybridization, but at this condition the M1 PNA probe lost the extended product M1. However, TEX&Hyb assay on PNA microchip is very specific and at adopted conditions is able to discriminate a single nucleotide substitution also.

3. Discussion

TEX amplification is based on the ability of polymerases to extend primers at incubation temperatures above the melting temperature of the DNA duplex formed by the primer and the template. The amplifications using single stranded cDNA of HCV and termination by ddNTP (or omission deoxynucleotide triphosphate) demonstrated a marked tendency of DeepVent(exo⁻) DNA polymerase to incorporate incorrect deoxynucleotides instead dideoxynucleotides or omitted deoxynucleotides. ThermoSequenase has very high fidelity for ddNTP incorporation, and the level of amplification achievable with this enzyme is approximately 300-fold, or at least 10 times higher than in published minisequencing assays (Dubiley, S., Kirillov, E. and Mirzabekov, A. (1999) Polymorphism analysis and gene detection by minisequencing on an array of gel-immobilized primers. Nucleic Acids Res., 27:e19). Czubayko M. (Czubayko, M. (2000) Herstellung der Thermus aquaticus DNA-Polymerase und Untersuchung ihrer dNTP-Substrataffinitat. PhD Thesis, Wolfsburg) demonstrated that template/primer dissociation constant for Taq polymerase is influenced by strength of the stacking interaction between the bases of the dNTP and the DNA and therefore by the sequence of the DNA. However, this explanation is not completely satisfactory, because different primer extension yields using TEX on the same sequence were observed using two templates of different length (38nt and 201nt).

TEX assays using termination at abasic sites, or at the 5′ end of templates, require additional steps for template preparation but produce a higher level of amplification (500- and 700-fold for DeepVent(exo⁻) and ThermoSequenase, respectively). The efficiency of TEX is similar for 5′ end and abasic site termination. Although the preparation of template with abasic site is simpler, the DNA polymerases can bypass the abasic sites (Kunkel, T. A., Shearman, C. W. and Loeb, L. A. (1981) Mutagenesis in vitro by depurination of phiX174 DNA. Nature, 291:349-351) and this event can decrease the level of amplification. Templates with 5′ end termination represent an attractive choice for optimized TEX assays and, moreover, there are many ways to generate templates with specific 5′ end sequence (multiprimer fragmentation, restriction endonucleases, cleavase and other).

DeepVent(exo⁻) DNA polymerase demonstrated the useful property of transcriptional termination at any of the for the four bases, proximal to a deoxyuridine and the next deoxynucleotide. The high efficiency of TEX on deoxyuridine is an attractive approach for TEX amplification designs. A limitation of this approach is that TEX is most efficient when the primers are extended only 3 bases. To achieve longer extensions with some efficiency, the template should have more than one deoxyuridine, and the distance between deoxyuridines should be less than 3 bases (FIG. 4).

It has been demonstrated that LNA (Ørum, H., Jakobsen, M. H., Koch, T., Vuust, J. and Borre, M. B. (1999) Detection of the Factor V Leiden Mutation by Direct Allele-specific Hybridization of PCR Amplicons to Photoimmobilized Locked Nucleic Acids. Clin Chem., 45:1898-1905) and PNA (Nielsen, P. E. (1999) An introduction to PNA. In: “Peptide Nucleic Acids: Protocols and Application”) have very high affinity to DNA and unique properties on discrimination of mismatched nucleotides (Ørum, H., Jakobsen, M. H., Koch, T., Vuust, J. and Borre, M. B. (1999) Detection of the Factor V Leiden Mutation by Direct Allele-specific Hybridization of PCR Amplicons to Photoimmobilized Locked Nucleic Acids. Clin Chem., 45:1898-1905). Furthermore, significant reduction in hybridization times can be achieved when using PNA probes (Nielsen, P. E. (1999) An introduction to PNA. In: “Peptide Nucleic Acids: Protocols and Application”). PNA is not used as substrate by DNA polymerase, and these properties make PNA an optimal probe for detection of TEX amplification products on microchip.

Incorporation of 9 to 11 bases will permit the use of longer probes on the array, making possible higher melting temperatures for immobilized TEX products. The disclosed TEX system can use at least 64 different primers in a single assay. This level of multiplexing enables detection of, for example, a large set of sequence variants. The TEX system can be easily adapted to a variety of signal readout systems, including detection on electronic (amperometric) devices. The detection limit can be improved using a signal amplification technique. For example, extension primers with haptens can be used in TEX and the haptens can facilitate signal amplification using rolling circle amplification (RCA) system (Guatelli, J. C, Whitfield, K. M., Kwoh, D. Y., Barringer, K. J., Richman, D. D. and Gingeras, T. R. (1990) Isothermal, in vitro amplification of nucleic acids by a multienzyme reaction modeled after retroviral replication. Proc. Natl. Acad. Sci. USA, 87:1874-1878; Lizardi, P. M., Huang, X., Zhu, Z., Bray-Ward, P, Thomas, D. and Ward, D. (1998) Mutation detection and single-molecule counting using isothermal rolling-circle amplification. Nature Genetics, 19:225-232; Schweitzer, B., Wiltshire, S., Lambert, J., O'Malley, S., Kukanskis, K., Zhu, Z., Kingsmore, S. F., Lizardi, P. M. and Ward, D. C. (2000) Immunoassays with rolling circle DNA amplification: a versatile platform for ultrasensitive antigen detection. Proc. Natl. Acad. Sci. USA, 97:10113-10119; Ladner, D. P., Leamon, J. H., Hamann, S., Tarafa, G., Strugnell, T., Dillon, D., Lizardi, P., Costa, J. (2001) Multiplex detection of hotspot mutations by rolling circle-enabled universal microarrays. Lab Invest., 81:1079-1086). Signal enhancement by RCA may improve the limit of detection by two to three orders of magnitude, and thus permit the detection of a few hundred viral genomes.

### B. References

Hevroni, D. and Livneh, Z. (1988) Bypass and termination at apurinic sites during replication of single-stranded DNA in vitro: a model for apurinic site mutagenesis. Proc. Natl. Acad. Sci. USA, 85:5046-5050.

Kunkel, T. A., Schaaper, R. M. and Loeb, L. A. (1983) Depurination-induced infidelity of deoxyribonucleic acid synthesis with purified deoxyribonucleic acid replication proteins in vitro. Biochemistry, 22:2378-2384.

Nallur, G., Luo, C., Fang, L., Cooley, S., Dave, V., Lambert, J., Kukanskis, K., Kingsmore, S., Lasken, R., and Schweitzer, B., Signal amplification by rolling circle amplification on DNA microarrays”, Nucleic Acids Res. 1;29(23):E118 (2001).

Walker, G. T., Little, M. C., Nadeau, J. G. and Shank, D. D. (1992) Isothermal in vitro amplification of DNA by restriction enzyme/DNA polymerase system. Proc. Natl. Acad. Sci. USA, 89:392-396.

Timofeev, E., Kochetkova, S. V., Mirzabekov, A. D. and Florentiev, V. L. (1996) Regioselective immobilization of short oligonucleotides to acrylic copolymer gels. Nucleic Acids Res., 24;3142-3148.

Burgener, M., Sanger, M., Candrian, U. (2000) Synthesis of a stable and specific surface plasmon resonance biosensor surface employing covalently immobilized peptide nucleic acids. Biocunjugate Chem., 11:749-754.

It is understood that the disclosed invention is not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a ”, “an”, and “the” include plural reference unless the context clearly dictates otherwise. Thus, for example, reference to “an extension primer” includes a plurality of such extension primers, reference to “the detection probe” is a reference to one or more detection probes and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. Publications cited herein and the material for which they are cited are specifically incorporated by reference. Nothing herein is to be construed as an admission that the invention is not entitled to antedate such disclosure by virtue of prior invention.

Those skilled in the art will recognize, or be able to ascertain using no more than routine experimentation, many equivalents to the specific embodiments of the invention described herein. Such equivalents are intended to be encompassed by the following claims.

We claim:

1. A method of amplifying nucleic acid sequences, the method comprising
bringing into contact one or more extension primers and one or more target templates and incubating under conditions that promote interaction of the extension primers and the target templates, extension of the extension primers using the interacting target templates as template, and dissociation of the extended extension primers from the target templates, whereby multiple extended extension primers are produced from at least one target template,
wherein the target templates each comprise a replication terminating feature.

2. The method of claim 1 wherein the extension primers and target templates are incubated under isothermal conditions.

3. The method of claim 1 wherein the extension primers and target templates are incubated under a single set of conditions.

4. The method of claim 1 wherein the target templates are nucleic acid sequences of interest.

5. The method of claim 1 wherein the extension primers each comprise a target complement portion.

6. The method of claim 5 wherein the extension primers each consist of a target complement portion.

7. The method of claim 5 wherein the extension primers each comprise nucleotides, wherein the nucleotides consist of the target complement portion.

8. The method of claim 5 wherein the extension primers each further comprise a non-target complement portion.

9. The method of claim 5 wherein the target complement portion of at least one of the extension primers has a melting temperature of about 5° C. to about 16° C. lower than the temperature at which the extension primers and target templates are incubated.

10. The method of claim 9 wherein the target complement portion of at least one of the extension primers has a melting temperature of about 10° C. to about 16° C. lower than the temperature at which the extension primers and target templates are incubated.

11. The method of claim 10 wherein the target complement portion of at least one of the extension primers has a melting temperature of about 11° C. to about 13° C. lower than the temperature at which the extension primers and target templates are incubated.

12. The method of claim 5 wherein the target complement portion of each extension primer has a melting temperature of about 10° C. to about 16° C. lower than the temperature at which the extension primers and target templates are incubated.

13. The method of claim 5 wherein the target complement portion of each extension primer has a calculated melting temperature of about 10° C. to about 16° C. lower than the temperature at which the extension primers and target templates are incubated.

14. The method of claim 5 wherein the target complement portion of at least one of the extension primers has a melting temperature of about 10° C. to about 16° C. lower than the temperature at which the extension primers and target templates are incubated, and wherein the extended extension primers produced by extension of the at least one extension primer has a melting temperature of about 0° C. to about 6° C. higher than the temperature at which the extension primers and target templates are incubated.

15. The method of claim 5 wherein the target complement portion of each of the extension primers has a melting temperature of about 10° C. to about 16° C. lower than the temperature at which the extension primers and target templates are incubated, and wherein each of the extended extension primers has a melting temperature of about 0° C. to about 6° C. higher than the temperature at which the extension primers and target templates are incubated.

16. The method of claim 5 wherein the melting temperature of the target complement portion of each of the extension primers is about 10° C. to about 22° C. lower than the melting temperature of the extended extension primer.

17. The method of claim 5 wherein the extension primers and target templates are incubated at about 68° C. to about 74° C., wherein the target complement portion of each extension primer has a melting temperature of about 55° C. to about 61° C.

18. The method of claim 17 wherein the extension primers and target templates are incubated at about 70° C. to about 72° C., wherein the target complement portion of each extension primer has a melting temperature of about 55° C. to about 61° C.

19. The method of claim 5 wherein the extension primers and target templates are incubated at about 68° C. to about 74° C., wherein the target complement portion of each extension primer has a melting temperature of about 55° C. to about 61° C., wherein the melting temperature of each extended extension primer is about 72° C. to about 77° C.

20. The method of claim 19 wherein the extension primers and target templates are incubated at about 70° C. to about 72° C., wherein the target complement portion of each extension primer has a melting temperature of about 55° C. to about 61° C., wherein the melting temperature of each extended extension primer is about 74° C. to about 76° C.

21. The method of claim 1 wherein at least one of the extended extension primers has a melting temperature of about 2° C. lower than to about 7° C. higher than the temperature at which the extension primers and target templates are incubated.

22. The method of claim 21 wherein at least one of the extended extension primers has a melting temperature of about 0° C. to about 6° C. higher than the temperature at which the extension primers and target templates are incubated.

23. The method of claim 22 wherein at least one of the extended extension primers has a melting temperature of about 2° C. to about 5° C. higher than the temperature at which the extension primers and target templates are incubated.

24. The method of claim 1 wherein the extended extension primers have melting temperatures of about 0° C. to about 6° C. higher than the temperature at which the extension primers and target templates are incubated.

25. The method of claim 1 wherein the extended extension primers have calculated melting temperatures of about 0° C. to about 6° C. higher than the temperature at which the extension primers and target templates are incubated.

26. The method of claim 1 wherein the extension primers and target templates are incubated at about 68° C. to about 74° C., wherein the melting temperature of each extended extension primer is about 72° C. to about 77° C.

27. The method of claim 26 wherein the extension primers and target templates are incubated at about 70° C. to about 72° C., wherein the melting temperature of each extended extension primer is about 74° C. to about 76° C.

28. The method of claim 1 wherein the extension primers are extended 5 to 11 nucleotides.

29. The method of claim 28 wherein the extension primers are extended 5 to 8 nucleotides.

30. The method of claim 28 wherein the extended extension primers have melting temperatures of about 0° C. to about 6° C. higher than the temperature at which the extension primers and target templates are incubated.

31. The method of claim 1 wherein the extended extension primers each comprise an extension portion, wherein the extension portion comprises nucleotides added to the extension primer by extension, wherein the extended extension primers each comprise an extended target complement portion, wherein the extended target complement portion comprises the target complement portion and the extension portion.

32. The method of claim 31 wherein the extended extension primers each further comprise a non-target complement portion.

33. The method of claim 32 wherein the extension primers each further comprise a non-target complement portion, wherein the non-target complement portion of an extended extension primer is the non-target complement portion of the extension primer from which the extended extension primer is produced.

34. The method of claim 31 wherein the extended extension primers each consist of an extended target complement portion.

35. The method of claim 31 wherein the extended extension primers each comprise nucleotides, wherein the nucleotides consist of the extended target complement portion.

36. The method of claim 31 wherein the target complement portion of each of the extension primers has a melting temperature of about 10° C. to about 16° C. lower than the temperature at which the extension primers and target templates are incubated, and wherein the extended target complement portion of each of the extended extension primers has a melting temperature of about 0° C. to about 6° C. higher than the temperature at which the extension primers and target templates are incubated.

37. The method of claim 31 wherein the melting temperature of the target complement portion of each of the extension primers is about 10° C. to about 22° C. lower than the melting temperature of the extended target complement portion of the extended extension primer.

38. The method of claim 31 wherein the extension primers and target templates are incubated at about 68° C. to about 74° C., wherein the target complement portion of each extension primer has a melting temperature of about 55° C. to about 61° C., wherein the melting temperature of the extended target complement portion of each extended extension primer is about 72° C. to about 77° C.

39. The method of claim 31 wherein the extended target complement portions of the extended extension primers have melting temperatures of about 0° C. to about 6° C. higher than the temperature at which the extension primers and target templates are incubated.

40. The method of claim 1 wherein at least 50 extended extension primers are produced from at least one of the target templates.

41. The method of claim 40 wherein at least 100 extended extension primers are produced from at least one of the target templates.

42. The method of claim 41 wherein at least 200 extended extension primers are produced from at least one of the target templates.

43. The method of claim 42 wherein at least 400 extended extension primers are produced from at least one of the target templates.

44. The method of claim 43 wherein at least 800 extended extension primers are produced from at least one of the target templates.

45. The method of claim 44 wherein at least 1000 extended extension primers are produced from at least one of the target templates.

46. The method of claim 45 wherein at least 2000 extended extension primers are produced from at least one of the,target templates.

47. The method of claim 1 wherein an average of at least 50 extended extension primers are produced from each of the target templates.

48. The method of claim 47 wherein an average of at least 100 extended extension primers are produced from each of the target templates.

49. The method of claim 48 wherein an average of at least 200 extended extension primers are produced from each of the target templates.

50. The method of claim 49 wherein an average of at least 400 extended extension primers are produced from each of the target templates.

51. The method of claim 50 wherein an average of at least 800 extended extension primers are produced from each of the target templates.

52. The method of claim 51 wherein an average of at least 1000 extended extension primers are produced from each of the target templates.

53. The method of claim 52 wherein an average of at least 2000 extended extension primers are produced from each of the target templates.

54. The method of claim 1 further comprising detecting one or more of the extended extension primers.

55. The method of claim 54 wherein each extended extension primer corresponds to one or more of the target templates, wherein the target templates correspond to nucleic acid sequences of interest, wherein detection of an extended extension primer indicates detection of the nucleic acid sequence of interest to which a target template corresponding to the detected extended extension primer corresponds.

56. The method of claim 54 wherein detection of the extended extension primers is performed simultaneously with incubation of the extension primers and target templates.

57. The method of claim 54 wherein the extension primers comprise a detection label, wherein the extended extension primers are detected via the detection label.

58. The method of claim 54 wherein the extended extension primers comprise a detection label, wherein the extended extension primers are detected via the detection label.

59. The method of claim 54 wherein the extended extension primers are detected via interaction of the extended extension primers with detection probes.

60. The method of claim 59 wherein the interaction is a base pairing interaction.

61. The method of claim 59 wherein the interaction is a hybridization interaction.

62. The method of claim 59 wherein the extended extension primers are covalently coupled to the detection probes.

63. The method of claim 62 wherein the extended extension primers are covalently coupled to the detection probes by ligation.

64. The method of claim 59 wherein the detection probes are stem-loop probes.

65. The method of claim 59 wherein the extended extension primers are covalently coupled to anchor probes, wherein covalent coupling of the extended extension primers is facilitated by the interaction of the extended extension primers with the detection probes.

66. The method of claim 65 wherein the anchor probes are associated with a substrate.

67. The method of claim 65 wherein the extended extension primers are covalently coupled to the anchor probes by ligation.

68. The method of claim 59 wherein the detection probes each comprise an oligonucleotide comprising modified nucleotides or an oligonucleotide analog, wherein the detection probes have a higher melting temperature than an oligonucleotide consisting of unmodified nucleotides having the same nucleotide base composition as the oligonucleotide comprising modified nucleotides or the oligonucleotide analog.

69. The method of claim 68 wherein the detection probes comprise peptide nucleic acid.

70. The method of claim 69 wherein the melting temperature of the extended extension primers interacting with the detection probes is at least 5° C. higher than the melting temperature of the extended extension primers interacting with the target templates.

71. The method of claim 69 wherein the melting temperature of the extended extension primers interacting with the detection probes is at least 10° C. higher than the melting temperature of the extended extension primers interacting with the target templates.

72. The method of claim 69 wherein the melting temperature of the extended extension primers interacting with the detection probes is at least 6° C. higher than the temperature at which the extension primers and target templates are incubated.

73. The method of claim 69 wherein the melting temperature of the extended extension primers interacting with the detection probes is at least 11° C. higher than the temperature at which the extension primers and target templates are incubated.

74. The method of claim 69 wherein the melting temperature of the extended extension primers interacting with the detection probes is at least 20° C. higher than the temperature at which the extension primers and target templates are incubated.

75. The method of claim 59 wherein detection probes each comprise a primer complement portion.

76. The method of claim 75 wherein the primer complement portion of the detection probes each comprise an oligonucleotide comprising modified nucleotides or an oligonucleotide analog, wherein the primer complement portions of the detection probes have a higher melting temperature than an oligonucleotide consisting of unmodified nucleotides having the same nucleotide base composition as the oligonucleotide comprising modified nucleotides or the oligonucleotide analog.

77. The method of claim 75 wherein the primer complement portion comprises peptide nucleic acid.

78. The method of claim 59 wherein the extended extension primers each comprise an extension portion, wherein the extension portion comprises nucleotides added to the extension primer by extension, wherein the extended extension primers each comprise an extended target complement portion and a non-target complement portion, wherein the extended target complement portion comprises the target complement portion and the extension portion.

79. The method of claim 78 wherein the detection probes interact with both the extended target complement portion and the non-target complement portion of the extended extension primers.

80. The method of claim 79 wherein the melting temperature of the extended extension primers interacting with the detection probes is at least 5° C. higher than the melting temperature of the extended extension primers interacting with the target templates.

81. The method of claim 79 wherein the melting temperature of the extended extension primers interacting with the detection probes is at least 10° C. higher than the melting temperature of the extended extension primers interacting with the target templates.

82. The method of claim 79 wherein the melting temperature of the extended extension primers interacting with the detection probes is at least 6° C. higher than the temperature at which the extension primers and target templates are incubated.

83. The method of claim 79 wherein the melting temperature of the extended extension primers interacting with the detection probes is at least 11 ° C. higher than the temperature at which the extension primers and target templates are incubated.

84. The method of claim 79 wherein the melting temperature of the extended extension primers interacting with the detection probes is at least 20° C. higher than the temperature at which the extension primers and target templates are incubated.

85. The method of claim 59 wherein the detection probes are associated with a substrate.

86. The method of claim 85 wherein the detection probes are attached to the substrate.

87. The method of claim 86 wherein the detection probes are covalently coupled to the substrate.

88. The method of claim 85 wherein the detection probes are indirectly associated with the substrate.

89. The method of claim 85 wherein the detection probes are directly associated with the substrate.

90. The method of claim 85 wherein the substrate is a single substrate structure, wherein all of the detection probes are associated with a single substrate.

91. The method of claim 90 wherein the substrate is a thin film, membranes, bead, microbead, bottle, dish, slide, fiber, optical fiber, woven fiber, chip, compact disk, shaped polymer, particles, or microparticle.

92. The method of claim 85 wherein the substrate is comprised of a plurality of substrate structures.

93. The method of claim 92 wherein the substrate comprises a plurality of beads.

94. The method of claim 93 wherein the beads are microbeads.

95. The method of claim 93 wherein the beads are paramagnetic beads.

96. The method of claim 85 wherein the substrate is acrylamide, cellulose, nitrocellulose, glass, gold, polystyrene, polyethylene vinyl acetate, polypropylene, polymethacrylate, polyethylene, polyethylene oxide, polysilicate, polycarbonate, teflon, fluorocarbon, nylon, silicon rubber, polyanhydride, polyglycolic acid, polylactic acid, polyorthoester, functionalized silane, polypropylfumerate, collagen, glycosaminoglycan, or polyamino acid.

97. The method of claim 1 wherein a plurality of extension primers are brought into contact with a plurality of target templates, wherein multiple extended extension primers are produced from each of a plurality of the target templates.

98. The method of claim 97 wherein the extension primers and target templates are incubated in the same reaction.

99. The method of claim 97 wherein the extension primers and target templates are incubated in a plurality of different reactions.

100. The method of claim 97 wherein each extension primer is incubated with the target templates in a different reaction.

101. The method of claim 97 wherein at least two extension primers are incubated with the target templates in a different reaction.

102. The method of claim 97 wherein the target templates each comprise a primer complement region, wherein the extension primers each comprise a target complement portion, wherein the target complement portion is complementary to a primer complement region of one or more of the target templates, wherein the extension primers comprise a set of extension primers, wherein each target complement portion of each extension primer in the set has similar hybrid stability.

103. The method of claim 102 wherein the extended extension primers comprise a set of extended extension primers, wherein each extended extension primer in the set has similar hybrid stability.

104. The method of claim 102 wherein the melting temperatures of each target complement portion of each extension primer in the set is within 5° C.

105. The method of claim 104 wherein the melting temperatures of each target complement portion of each extension primer in the set is within 3° C.

106. The method of claim 105 wherein the melting temperatures of each target complement portion of each extension primer in the set is within 2° C.

107. The method of claim 97 wherein the extended extension primers comprise a set of extended extension primers, wherein each extended extension primer in the set has similar hybrid stability.

108. The method of claim 107 wherein the melting temperatures of each extended extension primer in the set is within 7° C.

109. The method of claim 108 wherein the melting temperatures of each extended extension primer in the set is within 5° C.

110. The method of claim 109 wherein the melting temperatures of each extended extension primer in the set is within 3° C.

111. The method of claim 97 wherein the extended extension primers each comprise an extension portion, wherein the extension portion comprises nucleotides added to the extension primer by extension, wherein the extended extension primers each comprise an extended target complement portion, wherein the extended target complement portion comprises the target complement portion and the extension portion,
wherein the extended extension primers comprise a set of extended extension primers, wherein the extended target complement portion of each extended extension primer in the set has similar hybrid stability.

112. The method of claim 111 wherein the melting temperature of the extended target complement region of each extended extension primer in the set is within 7° C.

113. The method of claim 112 wherein the melting temperature of the extended target complement region of each extended extension primer in the set is within 5° C.

114. The method of claim 113 wherein the melting temperature of the extended target complement region of each extended extension primer in the set is within 3° C.

115. The method of claim 111 wherein the extended extension primers each further comprise a non-target complement region,
wherein each extended extension primer in the set has similar hybrid stability.

116. The method of claim 115 wherein the melting temperature of the extended extension primers is at least 5° C. higher than the melting temperature of the extended target complement portion of the extended extension primers.

117. The method of claim 115 wherein the melting temperature of the extended extension primers is at least 10° C. higher than the melting temperature of the extended target complement portion of the extended extension primers.

118. The method of claim 115 wherein the melting temperature of the extended extension primers is at least 6° C. higher than the temperature at which the extension primers and target templates are incubated.

119. The method of claim 115 wherein the melting temperature of the extended extension primers is at least 11° C. higher than the temperature at which the extension primers and target templates are incubated.

120. The method of claim 115 wherein the melting temperature of the extended extension primers is at least 20° C. higher than the temperature at which the extension primers and target templates are incubated.

121. The method of claim 97 further comprising detecting one or more of the extended extension primers.

122. The method of claim 121 wherein each extended extension primer corresponds to a different target template, wherein each target template corresponds to a different nucleic acid sequence of interest, wherein detection of an extended extension primer indicates detection of the nucleic acid sequence of interest to which the target template corresponding to the detected extended extension primer corresponds.

123. The method of claim 122 wherein detection of a plurality of extended extension primers indicates detection of the nucleic acid sequences of interest to which the target templates corresponding to the detected extended extension primers correspond.

124. The method of claim 121 wherein detection of the extended extension primers is performed simultaneously with incubation of the extension primers and target templates.

125. The method of claim 121 wherein the extension primers comprise a detection label, wherein the detection label of each extension primer is the same, wherein the extended extension primers are detected via the detection label.

126. The method of claim 121 wherein the extended extension primers comprise a detection label, wherein the detection label of each extended extension primer is the same, wherein the extended extension primers are detected via the detection label.

127. The method of claim 121 wherein the extension primers comprise a detection label, wherein the detection label of each extension primer is different, wherein the extended extension primers are detected via the detection label.

128. The method of claim 121 wherein the extended extension primers comprise a detection label, wherein the detection label of each extended extension primer is different, wherein the extended extension primers are detected via the detection label.

129. The method of claim 121 wherein a signal quotient is calculated for one or more of the detected extended extension primers.

130. The method of claim 121 wherein the extended extension primers are detected via detection labels, wherein a signal quotient is calculated for one or more of the detected extended extension primers using the detection labels.

131. The method of claim 121 wherein the extended extension primers are detected via detection labels, wherein the detection labels generate signals, wherein a signal quotient is calculated for one or more of the detected extended extension primers using the signals.

132. The method of claim 121 wherein the extended extension primers are detected via detection labels, wherein the detection labels generate signals, wherein the intensity of the signal generated for each detected extended extension primer is measured, wherein a signal quotient is calculated for one or more of the detected extended extension primers using the intensity of the measured signals.

133. The method of claim 132 wherein the signal quotient is a median signal quotient, wherein the median signal quotient of a given extended extension primer is calculated by dividing the signal intensity generated for that extended extension primer by the median signal intensity generated for the detected extended extension primers.

134. The method of claim 133 wherein the median signal quotient of a given extended extension primer is calculated by dividing the signal intensity generated for that extended extension primer by the median signal intensity generated for all of the detected extended extension primers.

135. The method of claim 133 wherein the median signal quotient of a given extended extension primer is calculated by dividing the signal intensity generated for that extended extension primer by the median signal intensity generated for a subset of all of the detected extended extension primers.

136. The method of claim 132 wherein the signal quotient is a ratio of signal quotients, wherein the ratio of signal quotients of a given extended extension primer is calculated by dividing the median signal quotient for that extended extension primer by the average signal quotient for that extended extension primer.

137. The method of claim 136 wherein each of the plurality of amplifications is carried out in the same way.

138. The method of claim 136 wherein the average signal quotient of a given extended extension primer is calculated by dividing the average signal intensity generated for that extended extension primer in 10 or more amplifications by the average signal intensity generated for the detected extended extension primers in the 10 or more amplifications.

139. The method of claim 136 wherein the average signal quotient of a given extended extension primer is calculated by dividing the average signal intensity generated for that extended extension primer in 20 or more amplifications by the average signal intensity generated for the detected extended extension primers in the 20 or more amplifications.

140. The method of claim 136 wherein the average signal quotient of a given extended extension primer is calculated by dividing the average signal intensity generated for that extended extension primer in 50 or more amplifications by the average signal intensity generated for the detected extended extension primers in the 50 or more amplifications.

141. The method of claim 136 wherein the average signal quotient of a given extended extension primer is calculated by dividing the average signal intensity generated for that extended extension primer in all of the amplifications by the average signal intensity generated for the detected extended extension primers in all of the amplifications.

142. The method of claim 136 wherein the average signal quotient of a given extended extension primer is calculated by dividing the average signal intensity generated for that extended extension primer in a subset of all of the amplifications by the average signal intensity generated for the detected extended extension primers in a subset of all of the amplifications.

143. The method of claim 121 wherein the extended extension primers are detected via interaction of the extended extension primers with detection probes, wherein each extended extension primer corresponds to a different detection probe, wherein each extended extension primer can interact with the detection probe to which the extended extension primer corresponds.

144. The method of claim 143 wherein the interaction is a base pairing interaction.

145. The method of claim 143 wherein the interaction is a hybridization interaction.

146. The method of claim 143 wherein the extended extension primers are covalently coupled to the detection probes.

147. The method of claim 146 wherein the extended extension primers are covalently coupled to the detection probes by ligation.

148. The method of claim 143 wherein the detection probes are stem-loop probes.

149. The method of claim 143 wherein the extended extension primers are covalently coupled to anchor probes, wherein covalent coupling of the extended extension primers is facilitated by the interaction of the extended extension primers with the detection probes, wherein each extended extension primer corresponds to a different anchor probe.

150. The method of claim 149 wherein the anchor probes are associated with a substrate.

151. The method of claim 149 wherein the extended extension primers are covalently coupled to the anchor probes by ligation.

152. The method of claim 143 wherein the detection probes comprise peptide nucleic acid.

153. The method of claim 152 wherein the melting temperature of the extended extension primers interacting with the detection probes to which the extended extension primers correspond is at least 5° C. higher than the melting temperature of the extended extension primers interacting with the target templates to which the extended extension primers correspond.

154. The method of claim 152 wherein the melting temperature of the extended extension primers interacting with the detection probes to which the extended extension primers correspond is at least 10° C. higher than the melting temperature of the extended extension primers interacting with the target templates to which the extended extension primers correspond.

155. The method of claim 152 wherein the melting temperature of the extended extension primers interacting with the detection probes to which the extended extension primers correspond is at least 6° C. higher than the temperature at which the extension primers and target templates are incubated.

156. The method of claim 152 wherein the melting temperature of the extended extension primers interacting with the detection probes to which the extended extension primers correspond is at least 11° C. higher than the temperature at which the extension primers and target templates are incubated.

157. The method of claim 152 wherein the melting temperature of the extended extension primers interacting with the detection probes to which the extended extension primers correspond is at least 20° C. higher than the temperature at which the extension primers and target templates are incubated.

158. The method of claim 143 wherein detection probes each comprise a primer complement region.

159. The method of claim 158 wherein the primer complement region comprises peptide nucleic acid.

160. The method of claim 143 wherein the detection probes are associated with a substrate.

161. The method of claim 160 wherein the detection probes are attached to the substrate.

162. The method of claim 161 wherein the detection probes are covalently coupled to the substrate.

163. The method of claim 160 wherein the detection probes are indirectly associated with the substrate.

164. The method of claim 160 wherein the detection probes are directly associated with the substrate.

165. The method of claim 160 wherein the substrate is a single substrate structure, wherein all of the detection probes are associated with a single substrate.

166. The method of claim 165 wherein the detection probes are each associated with a different region of the substrate.

167. The method of claim 166 wherein the region of the substrate where an extended extension primer is detected is indicative of the identity of the detected extended extension primer.

168. The method of claim 165 wherein the substrate is a thin film, membranes, bead, microbead, bottle, dish, slide, fiber, optical fiber, woven fiber, chip, compact disk, shaped polymer, particles, or microparticle.

169. The method of claim 160 wherein the substrate is comprised of a plurality of substrate structures.

170. The method of claim 169 wherein the detection probes are each associated with a different substrate structure.

171. The method of claim 170 wherein the substrate structure on which an extended extension primer is detected is indicative of the identity of the detected extended extension primer.

172. The method of claim 169 wherein the detection probes are each associated with a different region of a substrate structure, a different substrate structure, or a different region of a different substrate structure.

173. The method of claim 172 wherein the substrate structure and region of the substrate structure where an extended extension primer is detected is indicative of the identity of the detected extended extension primer.

174. The method of claim 169 wherein the substrate comprises a plurality of beads.

175. The method of claim 174 wherein the beads are microbeads.

176. The method of claim 174 wherein the beads are paramagnetic beads.

177. The method of claim 160 wherein the substrate is acrylamide, cellulose, nitrocellulose, glass, gold, polystyrene, polyethylene vinyl acetate, polypropylene, polymethacrylate, polyethylene, polyethylene oxide, polysilicate, polycarbonate, teflon, fluorocarbon, nylon, silicon rubber, polyanhydride, polyglycolic acid, polylactic acid, polyorthoester, functionalized silane, polypropylfumerate, collagen, glycosaminoglycan, or polyamino acid.

178. The method of claim 1 wherein the target templates each comprise a primer complement region, an extension region, and replication terminating feature.

179. The method of claim 178 wherein the extension primers each comprise a target complement portion, wherein the target complement portion is complementary to a primer complement region of one or more of the target templates.

180. The method of claim 178 wherein the replication terminating feature is a 5′ end, one or more abasic nucleotides, or one or more derivatized nucleotides.

181. The method of claim 180 wherein the replication terminating feature is a 5′ end.

182. The method of claim 181 wherein the target templates correspond to nucleic acid molecules comprising nucleic acid sequences of interest, wherein the replication terminating feature of at least one of the target templates is produced by cleavage of the nucleic acid molecule to which the target template corresponds.

183. The method of claim 192 wherein the cleavage is accomplished using one or more restriction endonucleases, cleavase I, T4 endonuclease, endonuclease IV, one or more resolvases, or a combination.

184. The method of claim 182 wherein the nucleic acid molecule is DNA containing deoxyuridine, wherein the cleavage is accomplished by excising uracil residues in the nucleic acid molecule using uracil DNA glycosylase and cleaving the backbone of the nucleic acid molecule with endonuclease.

185. The method of claim 184 wherein the uracil residues are excised in the presence of single stranded binding protein, wherein excision of uracil in loops of the nucleic acid molecule is favored over excision of uracil in single-stranded regions of the nucleic acid molecule.

186. The method of claim 184 wherein the DNA containing deoxyuridine is produced by replicating a nucleic acid molecule using one or more DNA primers containing one or more deoxyuridines.

187. The method of claim 182 wherein one or more mismatch probes are hybridized to the nucleic acid molecule, wherein there is at least one mismatch between each mismatch probe and the nucleic acid molecule, wherein the cleavage is accomplished by using cleavase I, wherein cleavase I cleaves at the mismatches.

188. A method of detecting nucleic acid sequences, the method comprising bringing into contact one or more extension primers and one or more target templates and incubating under conditions that promote interaction of the extension primers and the target templates, extension of the extension primers using the interacting target templates as template, and dissociation of the extended extension primers from the target templates, whereby multiple extended extension primers are produced from at least one target template, and
detecting one or more of the extended extension primers.

189. The method of claim 188 wherein each extension primer corresponds to one or more of the target templates, wherein the target templates correspond to nucleic acid sequences of interest, wherein detection of an extended extension primer indicates detection of the nucleic acid sequence of interest to which a target template corresponding to the detected extended extension primer corresponds.

190. A method of detecting nucleic acid sequences, the method comprising
bringing into contact one or more extension primers and one or more target templates and incubating under conditions that promote interaction of the extension primers and the target templates, extension of the extension primers using the interacting target templates as template, and dissociation of the extended extension primers from the target templates, whereby multiple extended extension primers are produced from at least one target template, and
detecting one or more of the extended extension primers.

191. The method of claim 190 wherein each extension primer corresponds to one or more of the target templates, wherein the target templates correspond to nucleic acid sequences of interest, wherein detection of an extended extension primer indicates detection of the nucleic acid sequence of interest to which a target template corresponding to the detected extended extension primer corresponds.

192. A method of amplifying nucleic acid sequences, the method comprising
bringing into contact one or more extension primers and one or more target templates and incubating under conditions that promote interaction of the extension primers and the target templates, extension of the extension primers using the interacting target templates as template, and dissociation of the extended extension primers from the target templates, whereby multiple extended extension primers are produced from at least one target template.


SEQUENCE LISTING (37 sequences)

SEQ ID NO: 1 - DNA, 39 bp, Artificial Sequence
  Description of Artificial Sequence/note = synthetic construct
ttttacctcccggggcactcgcaagcaccctatcaggca

SEQ ID NO: 2 - DNA, 47 bp, Artificial Sequence
  misc_feature 4,9,11,19: n = deoxyuridines
cacnccccngngaggaacnactgtcttcacgcagaaagcgtctagcc

SEQ ID NO: 3 - DNA, 58 bp, Artificial Sequence
  misc_feature 2,7,8,11,13,17: n = deoxyuridines
anggcgnnagnangagngtcgtgcagcttccaggaccccccctcccgggagagccata

SEQ ID NO: 4 - DNA, 26 bp, Artificial Sequence
  Description of Artificial Sequence/note = synthetic construct
ctagacgctttctgcgtgaagacagt

SEQ ID NO: 5 - DNA, 21 bp, Artificial Sequence
  Description of Artificial Sequence/note = synthetic construct
gggtcctggaagctgcacgac

SEQ ID NO: 6 - DNA, 40 bp, Artificial Sequence
  Description of Artificial Sequence/note = synthetic construct
gactgctagccgagtagtgttgggtcgcgaaaggccttgt

SEQ ID NO: 7 - DNA, 25 bp, Artificial Sequence
  Description of Artificial Sequence/note = synthetic construct
gccatggcgttagtatgagtgtcgt

SEQ ID NO: 8 - DNA, 25 bp, Artificial Sequence
  Description of Artificial Sequence/note = synthetic construct
aaggcctttcgcgacccaacactac

SEQ ID NO: 9 - DNA, 39 bp, Artificial Sequence; identical to SEQ ID NO: 1
  Description of Artificial Sequence/note = synthetic construct

SEQ ID NO: 10 - DNA, 65 bp, Artificial Sequence
  Description of Artificial Sequence/note = synthetic construct
cgacatacgaggatagcagacagatgactagtcgttattaccgactagtcatctgtctgctatcc

SEQ ID NO: 11 - DNA, 67 bp, Artificial Sequence
  Description of Artificial Sequence/note = synthetic construct
cgacatacgaggatagcagacagatgaactagtcgttattaccgactagttcatctgtctgctatcc

SEQ ID NO: 12 - RNA, 18 nt, Artificial Sequence
  Description of Artificial Sequence/note = synthetic construct
uggucugcggaaccggug

SEQ ID NO: 13 - RNA, 18 nt, Artificial Sequence
  Description of Artificial Sequence/note = synthetic construct
gcccccgcgagacugcua

SEQ ID NO: 14 - RNA, 18 nt, Artificial Sequence
  Description of Artificial Sequence/note = synthetic construct
gcccccgcgagacugcua

SEQ ID NO: 15 - DNA, 110 bp, Artificial Sequence
  Description of Artificial Sequence/note = synthetic construct
tagatcactcccctgtgaggaactactgtcttcacgcagaaagcgtctagccatggcgttagtatgagtgtcgtgcagcttccaggaccccccctcccgggagagccata

SEQ ID NO: 16 - DNA, 26 bp, Artificial Sequence
  Description of Artificial Sequence/note = synthetic construct
catgacatcaacccgctcaatgcctg

SEQ ID NO: 17 - DNA, 22 bp, Artificial Sequence
  Description of Artificial Sequence/note = synthetic construct
aggcattgagcgggttgatgtc

SEQ ID NO: 18 - DNA, 40 bp, Artificial Sequence; identical to SEQ ID NO: 6
  Description of Artificial Sequence/note = synthetic construct

SEQ ID NO: 19 - DNA, 40 bp, Artificial Sequence
  Description of Artificial Sequence/note = synthetic construct
gactgccagccgagtagtgttgggtcgcgaaaggccttgt

SEQ ID NO: 20 - DNA, 20 bp, Artificial Sequence
  Description of Artificial Sequence/note = synthetic construct
ctttcgcgacccaacactac

SEQ ID NO: 21 - DNA, 26 bp, Artificial Sequence
  Description of Artificial Sequence/note = synthetic construct
atctccaggcattgagcgggttaatc

SEQ ID NO: 22 - DNA, 26 bp, Artificial Sequence
  Description of Artificial Sequence/note = synthetic construct
atctctaggcattgagcgggttaatc

SEQ ID NO: 23 - DNA, 40 bp, Artificial Sequence
  Description of Artificial Sequence/note = synthetic construct
actacacttgatcttcccaacccgctcaatgcctggagat

SEQ ID NO: 24 - DNA, 39 bp, Artificial Sequence
  misc_feature 1,4,6,17: n = deoxyuridines
nggncngcggaaccggngagtacaccggaattgccagga

SEQ ID NO: 25 - DNA, 40 bp, Artificial Sequence
  misc_feature 14,17: n = deoxyuridines
gcccccgcgagacngcnagccgagtagtgttgggtcgcga

SEQ ID NO: 26 - DNA, 40 bp, Artificial Sequence
  misc_feature 10,13,14,15,17: n = deoxyuridines
acgaccgggnccnnncntggatcaacccgctcaatgcctg

SEQ ID NO: 27 - DNA, 40 bp, Artificial Sequence; identical to SEQ ID NO: 26
  misc_feature 10,13,14,15,17: n = abasic nucleotide

SEQ ID NO: 28 - DNA, 19 bp, Artificial Sequence
  Description of Artificial Sequence/note = synthetic construct
tcctggcaattccggtgta

SEQ ID NO: 29 - DNA, 23 bp, Artificial Sequence
  Description of Artificial Sequence/note = synthetic construct
tcgctacccaacactactcggct

SEQ ID NO: 30 - DNA, 26 bp, Artificial Sequence
  Description of Artificial Sequence/note = synthetic construct
aggcattgagcgggttgatccaagaa

SEQ ID NO: 31 - DNA, 24 bp, Artificial Sequence
  Description of Artificial Sequence/note = synthetic construct
cccaacactactcggctggcagtc

SEQ ID NO: 32 - DNA, 26 bp, Artificial Sequence
  Description of Artificial Sequence/note = synthetic construct
gttccgcagaccactatggctctccc

SEQ ID NO: 33 - DNA, 26 bp, Artificial Sequence
  Description of Artificial Sequence/note = synthetic construct
gttccccagaccactatggctctccc

SEQ ID NO: 34 - DNA, 24 bp, Artificial Sequence
  Description of Artificial Sequence/note = synthetic construct
gagagccatagtggtctgcggaac

SEQ ID NO: 35 - DNA, 24 bp, Artificial Sequence
  Description of Artificial Sequence/note = synthetic construct
gagagccatagtggtctggggaac

SEQ ID NO: 36 - DNA, 22 bp, Artificial Sequence
  Description of Artificial Sequence/note = synthetic construct
aacccgctcaatgcctagagat

SEQ ID NO: 37 - DNA, 24 bp, Artificial Sequence
  Description of Artificial Sequence/note = synthetic construct
cccaacactactcggctagcagtc